(19) United States
(12) Patent Application Publication
(10) Pub. No.: US 2010/0285490 A1
(43) Pub. Date: Nov. 11, 2010

(54) DETECTION APPARATUS

(75) Inventors: Robert Dees, San Diego, CA (US); Dale Dembrow, Damascus, MD (US); Brent Henry, Boyds, MD (US); Espir Kahatt, Carlsbad, CA (US); James Meegan, Woodbine, MD (US); Timothy Powers, Carlsbad, CA (US); Steven Roman, Carlsbad, CA (US); Jeffrey Rossio, Frederick, MD (US)

Correspondence Address: LIFE TECHNOLOGIES CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 11/966,747

(22) Filed: Dec. 28, 2007

Related U.S. Application Data
(60) Provisional application No. 60/882,895, filed on Dec. 29, 2006.

(51) Int. Cl. G01N 33/53; C12M 1/34

(52) U.S. Cl. 435/7.1; 435/287.2

(57) ABSTRACT

The present invention relates to, in part, methods, reagents and apparatuses for the detection of agents. The present invention also relates, in part, to compositions including, but not limited to, flow cells, assay chambers, reagent reservoir delivery units and devices for holding an assay chamber. The present invention also provides various components and combinations of components for various detection apparatuses. The present invention also relates to a portable agent detection apparatus that can be used in the field or at a point of care and is not limited to specialized laboratories or limited to use by highly skilled users.

This application claims priority to U.S. Provisional Application No. 60/882,895 filed Dec. 29, 2006, which is incorporated herein by reference in its entirety.

This invention was made with Government support under contract HDTRA1-04-C-0047 awarded by the Defense Threat Reduction Agency (DTRA). The Government has certain rights in this invention.

### 1. FIELD OF THE INVENTION

The present invention provides, in part, methods, reagents and apparatuses for the detection of agents. The present invention also provides, in part, components for a detection apparatus including, but not limited to, flow cells, assay chambers and assay chamber clamps. The present invention also provides, in part, various components and combinations of components for various detection apparatuses.

### 2. BACKGROUND OF THE INVENTION

There are many uses for detection devices. Examples include the detection of pollutants, infectious agents, plant pathogens, toxins, bioweapons, etc. Most current detection devices are located at a central location and samples are transported to the central location for analysis.

Various assay methods for detecting molecules in a sample have been investigated over the years. Most of these methods involve specialized equipment that is not easily portable and/or constructed for use in the field or at a point-of-care. Many of the methods and equipment currently in practice require components that are not compatible with the conditions experienced in the field, for example temperatures, bumping and shaking, dust, insects, etc. Additionally, many of these methods in the art and the operation of related equipment require a highly trained and or educated person.

While successful for analytes that occur at relatively high concentrations (e.g., blood glucose), developing point-of-care tests for low abundance target molecules can be problematic. This difficulty is largely attributable, at least in part, to combining two mutually antagonistic product requirements: (1) the need for sophisticated technology to meet demanding test specifications including ultra-sensitivity and (2) the need for low cost, user-friendly, and portable tests that can be operated by unskilled operators.

Therefore, there remains a need for detection apparatuses that are portable, easy to use, able to be used by personnel with minimal training or related education, utilize rapid detection assays, do not require specialized laboratories, are cost effective and/or are in some instances capable of detecting low levels of an agent(s). The present invention meets this and other needs.

Citation or discussion of a reference herein shall not be construed as an admission that such is prior art to the present invention.

### 3. SUMMARY OF THE INVENTION

The present invention relates, in part, to methods, reagents and apparatuses for the detection of agents. The present invention also provides, in part, components for a detection apparatus including, but not limited to, flow cells, assay chambers and assay chamber clamps. The present invention also provides, in part, various components and combinations of components for various detection apparatuses, as well as the apparatuses themselves.

Detection apparatuses and/or assays of the invention provide methods for detecting an agent or agents of interest. Some detection apparatuses of the invention provide a platform for detecting essentially an unlimited number of agents. In some embodiments, the agent or agents detected by an apparatus of the invention are determined using a removable assay chamber (e.g., a flow cell) and/or the assay reagents. One advantage and convenience of having one apparatus, such as this, is that it can be utilized to detect a wide range of agents, by changing the assay chamber and/or the assay reagents.

The present invention, in part, provides agent detection apparatuses, assay chambers and related methods. These apparatuses and/or assay chambers can be used to detect, analyze, identify, and/or quantitate an agent in a sample(s). Therefore, the invention also provides methods of detect, analyze, identify, and/or quantitate an agent in a sample(s). In some embodiments, an agent detection apparatus comprises at least one component selected from the group consisting of an assay chamber, a light source, a detection device, a computer, a global positioning system receiver, at least one pump for fluids, a reagent pack holder, a reagent pack, a power source (e.g., a DC power source such as a battery), a plug for drawing electrical current, image analysis software, and an assay chamber (e.g., a flow cell) clamp device. In some embodiments, an agent detection apparatus comprises a graphic user interface. In some embodiments, a detection apparatus of the invention is portable by an average person.

The present invention also provides various assay chambers and/or reactive surfaces for performing assay methods of the invention. In some embodiments, an assay chamber comprises at least one binding molecule or population of binding molecules. In some embodiments, an assay chamber comprises at least two different binding molecules for the detection of at least two different agents. In some embodiments, an assay chamber is a flow cell. In some embodiments, an assay chamber comprises multiple channels or subchambers.

In some embodiments, an assay chamber comprises multiple sites for detecting multiple agents in a sample. In some embodiments, an assay chamber comprises a waveguide element. In some embodiments, an assay chamber is designed so the assay reagents, including a sample, can be re-circulated or looped over a detection region(s), situs and/or capture binding molecule(s). In some embodiments, an assay chamber comprises channels wherein each channel comprises at least two ports for introducing, removing and/or circulating assay reagents. In some embodiments, an assay chamber comprises a port for introducing a sample. In some embodiments, this port is compatible with a syringe. In some embodiments, this port is equipped with a one way valve. In some embodiments, an assay chamber comprises multiple channels, e.g., as shown in FIG. 11. In some embodiments, a channel is utilized for sample analysis. In some embodiments, a channel is utilized for a positive control(s) for an assay. In some embodiments, a channel is utilized for a negative control(s) for an assay.

Some detection apparatuses, assay chambers and related methods of the invention are utilized to detect, analyze, identify, and/or quantitate binding interaction between at least two molecules (e.g., a capture binding molecule and an agent which binds to the capture binding molecule). Some detection apparatuses assay chambers and related methods utilize arrays, e.g., protein arrays, receptor arrays, nucleic acid arrays. Protein arrays include cellular receptor arrays (e.g., cell membrane, nuclear and other types of cell receptors) and antibody arrays. Some of these arrays are useful for drug and or ligand screening.

Nucleic acid arrays include arrays for SNPs (single nucleotide polymorphisms), cDNA arrays, oligonucleotide arrays, plasmid arrays, etc. In some embodiments, a nucleic acid array is utilized to identify a corresponding cell type, organism, virus, bacteria, or fungus. Samples can be contacted with a nucleic acid array or a sample can undergo an amplification step prior to contact with a nucleic acid array. Amplification steps include, but are not limited to, nucleic acid amplification and/or amplification of the agent itself, e.g., culturing or amplifying a virus, bacterium, fungus, cell or organism. Nucleic acid amplification includes, but is not limited to, a polymerase chain reaction method (PCR), a ligase chain reaction (LCR), self sustained sequence replication (3SR), nucleic acid sequence-based amplification (NASBA), the use of Q Beta replicase, reverse transcription, nick translation, and the like.

In some embodiments, a detection apparatus of the invention comprises a device for holding an assay chamber of the invention. In some embodiments, an assay chamber holding device is a clamping device. In some embodiments, an assay chamber holding device comprises a switch that is triggered when an assay chamber is correctly inserted. This switch can produce a signal to a user and/or computer indicating proper or improper placement. This switch may also not allow an assay to be conducted unless an assay chamber is detected to be correctly inserted. In some embodiments, an assay chamber holding device creates a leak proof seal(s) with the assay chamber such as via connection comprising O-rings. FIG. 14 shows an exemplary assay chamber holding device of the invention.

In some embodiments, a detection apparatus can be utilized to detect one or more agents at a time from one sample or more than one sample. Some embodiments of the invention utilize a sandwich type assay or capture assay. For example, a capture binding molecule(s) is attached to a surface, wherein the surface binding molecule has binding affinity for an agent(s). In some embodiments, an agent bound (“captured”) by the capture binding molecule(s) is bound to a second binding molecule(s), essentially sandwiching it between the first and second binding molecule. In some embodiments, the second binding molecule is directly or indirectly labeled with a detectable label (e.g., a LSL, a fluorophore or a nanocrystal).

In some embodiments, an assay produces a detectable signal selected from the group consisting of light scattering, luminescence, fluorescence or combinations thereof In some embodiments, a detectable signal is generated from a detectable label attached directly to an agent(s) or detector binding molecule. In some embodiments, a detectable label is utilized as described herein. In some embodiments, at least two detectable signals are produce, wherein the at least two signals are distinguishable. Typically, each of the at least two distinguishable signals represent a different agent or event (e.g., binding event).

In some embodiments, a detection apparatus or method utilizes at least one light scattering label (LSL). In some embodiments, an assay using light scattering also utilizes a liquid absorbing member (LAM). Some embodiments utilize an evanescent wave or waveguide in combination with LSLs to detect an agent(s).

In some embodiments, a detectable label is a nanocrystal. In some embodiments, a nanocrystal is a semiconductor nanocrystals or quantum dot.

In some embodiments, a sample(s) is processed prior to agent detection analysis. For example a sample may undergo a process for amplifying, concentrating and/or purifying (partially or completely) an agent(s) of interest, if present, in a sample. In some embodiments, concentration and/or purification involves utilizing particles that bind to an agent(s). Particles include, but are not limited to, non-magnetic, magnetic, paramagnetic or magnetic. In some embodiments, particles are comprised of a binding molecule(s) that binds the agent of interest. In some embodiments, the particles are beads.

In some embodiments, a detection apparatus of the invention utilizes a computer to control a process(es) or mechanics of an assay. A computer can be utilized for at least one of the following functions: (1) controlling part or all of the assay steps including sample injection/introduction; (2) controlling assay reagent introduction to a sample chamber; (3) controlling recirculation of an assay reagent including recirculation of a sample; (4) controlling the temperature of assay conditions, assay reagents, sample storage, and/or any component(s) of a detection apparatus of the invention; (5) acquiring data from a detection device; (6) analyzing, compiling, or interpreting data; (7) recording and associating a GPS position with a sample(s); and/or (8) providing information to a user such as instructions, information relevant to an agent, warnings, and/or results. In some embodiments, a graphic user interface (GUI) maybe utilized, e.g., to prompt a user to do certain steps, for data input such as sample identification, to provide which step of an assay is currently being performed, and/or to notify a user that a sample is positive, negative and/or contains a certain level of an agent.

The present invention also provides reagent packs, e.g., for assays of the invention as described herein. Reagent packs typically comprise at least one reservoir, chamber, tube, etc., e.g., containing an assay reagent. In some embodiments, a reagent pack comprises more than one reservoir, chamber, tube, etc., e.g., containing an assay reagent. In some embodiments, a reagent pack comprises all of the assay reagents for a particular assay(s) and/or for an assay chamber. In some embodiments, a reagent pack comprises a computer readable label. In some embodiments, a reagent pack comprises a label or marking that matches or corresponds to a compatible assay or assay chamber. In some embodiments, a reagent pack comprises at least one reservoir, chamber, tube, etc., for the collection of liquids, e.g., assay waste reagents, sometimes referred to as a waste reservoir. In some embodiments, a waste reservoir comprises a substance that solidifies and/or absorbs liquids, e.g., a gel forming powder or a sponge. In some embodiments, a waste reservoir comprises a substance for rendering a reagent less hazardous or non-hazardous. This can include a decontaminating compound, e.g., that inactivates a pathogen or converts a toxic compound to a less toxic form or to a less toxic compound. In some embodiments, a reagent pack is designed to fit into a compartment of a detection apparatus of the invention. In some embodiments, this compartment automatically inserts a port into at least one reservoir of the reagent pack. In some embodiments, a reagent pack is a blister pack.

In some embodiments, a reagent or cleaning pack comprises at least one of the following components selected from the group consisting of a blocking solution (e.g., comprising 1% w/v Casein in PBS; an antibody; a labeled antibody; glycerol (e.g., a 50% glycerol solution; a cleaning and/or disinfecting solution; a wash solution (e.g., water) and an absorptive material. In some embodiments, a reagent of a reagent pack comprises Kathon (e.g., from Sigma-Aldrich Corp., St. Louis, Mo.) as a preservative.

In some embodiments, a reagent pack is a cleaning pack. A cleaning pack comprises solutions for cleaning, decontaminating, sanitizing and or disinfecting the system or parts of the system. In some embodiments, at least one reservoir of a cleaning pack comprises a detergent. In some embodiments, at least one reservoir of a cleaning pack comprises at least one compound or material selected from the group consisting of an alcohol, a sodium hypochlorite, a quaternary ammonium compound or material similar to these. In some embodiments, at least one reservoir of a cleaning pack comprises a compound(s) selected from the group consisting of a chlorine containing disinfectant (e.g., sodium hypochlorite); a stabilized chlorine dioxide, a phenol, a chlorhexidine gluconate, a quaternary ammonium compound, a glutaraldehyde, an alcohol, an iodine containing compound, a pine oil, a 5-Bromo-5-Nitro-1,3-Dioxane (e.g., 1% 5-Bromo-5-Nitro-1,3-Dioxane in water) or a mercury compound. Some decontamination, sanitizing, and/or deactivating compound(s) that could be used are commercially available including, but not limited to, Wescodyne® (Steris, Mentor, Ohio) or Cidex® (Advanced Sterilization Products, Irvine, Calif.). In some embodiments, all of the reservoirs will comprise a cleaning, decontaminating, sanitizing and or disinfecting material or solution.

The present invention also provides various kits related to the assays and detection apparatuses of the invention as described herein. In some embodiments, kits may include one or more of the following: an assay reagent, combinations of assay reagents, all necessary reagents for an assay, a sample buffer, a wash buffer, a decontamination liquid or buffer, a labeled binding molecule(s), an unlabeled binding molecule(s), a control reagent(s) (e.g., positive and/or negative control samples), a reagent pack, an assay chamber (e.g., interchangeable), a detection apparatus, a manual, instructions, personal protective gear (such as gloves, a suit (e.g., Tyvek® suit), a respirator, a self contained breathing apparatus, safety glasses), software, sample collection containers (e.g., tubes, boxes, syringes), or a syringe (e.g., for inputting a sample into an assay chamber or detection apparatus).

Additionally, the invention provides various related business methods as described herein.

The present invention provides numerous benefits, including, in at least some embodiments, a single assay for multiple agents, a detection apparatus for analyzing and detection numerous agents, a portable detection apparatus, reagent packs, reagent pack delivery units, low fluid volumes consumption which is beneficial for, e.g., environmental pollution (less waste); lower costs of expensive reagents and less sample fluid is used for assays; compactness of the system(s), e.g., due to integration of functionality and small volumes; sensitive levels of agent detection and/or detecting binding interactions; relatively low fabrication costs; and/or fabrication in mass production.

### 4. BRIEF DESCRIPTION OF THE FIGURES

For the purpose of illustrating the invention, there are depicted in the drawings certain embodiments on the invention. However, the invention is not limited to the precise arrangements and instrumentalities of embodiments depicted in the drawings.

FIG. 1 is a bottom view of an exemplary detection apparatus of the present invention shown with the bottom of the case removed to show some of the internal components.

FIG. 2 is a bottom view of an exemplary detection apparatus of the present invention shown with the battery, lid and several parts from the Digital View Box (DVB) removed to show some of the components. The DVB consists of a camera, a focusing lens, a fiber optic light pipe and line array, and a flow cell clamp. All of these are housed in a robust enclosure (e.g., comprising a metal such as aluminum), and can be manipulated as a single unit.

FIG. 3 is a top view of an exemplary detection apparatus of the present invention shown with the lid of the case off. This is a view of the panel of the instrument as seen by the user, and identifies the components of a DVB. They are a camera body 340, a focusing lens 330, a flow cell compartment 320, a flow cell clamping device 310, and a flow cell clamp handle 300. In the shown embodiment, a flow cell is inserted from the top into the DVB, where it resides during the assay.

FIG. 4 is a top view of an exemplary detection apparatus of the present invention shown with the lid of the case open.

FIG. 5 is a side view of an exemplary detection apparatus of the present invention shown without the case to show some of the components.

FIG. 6 is an example of an assay format of the present invention. In this embodiment, a first antibody (e.g., capture antibody) is attached or associated to a surface (e.g., a planar array). An agent (diamond shape) is bound to the first antibody. A second antibody is bound to the agent, wherein the second antibody is linked to a tag (triangle shape) such as biotin. A third antibody binds the tag (e.g., the third antibody binds biotin), wherein the third antibody is associated or bound to a label (circle shape) such as a gold particle (e.g., 80 nm). This is an example of a sandwich assay using a direct capture binding molecule and indirect detection.

FIG. 7 is an example of a graphic user interface (GUI) of the present invention.

FIG. 8 is an example of a GUI of the present invention.

FIG. 9 is a schematic representation of an exemplary system for providing a product to a party.

FIG. 10 is a schematic representation of an exemplary system for advising a party as to the availability of a product.

FIG. 11 is a schematic representation of an exemplary assay chamber (e.g., a flow cell). FIG. 11A shows an assembled flow cell. FIG. 11B shows 3 components of the flow cell. FIG. 11C is a cross sectional view showing, inter alia, 3 channels. Please note FIG. 11C is not necessarily drawn to scale.

FIG. 12 shows an exemplary flow cell with a syringe attached to a sample port.

FIG. 13 shows a close-up view of a flow cell antigen-capture area. It consists in this instance of 18 discrete windows, each framing a number of individual array spots. The crosses are fiduciary marks for alignment.

FIG. 14 shows an example of a clamp for an assay chamber (e.g., a flow cell clamp) in an assembled form and with the parts as disassembled.

FIG. 15 shows an exemplary flow chart for an exemplary assay. This example may be applied to a bioscreening application. The flow sheet concentrates on the initiation steps taken by the user.

FIG. 16 shows an exemplary brief outline of a type of assay that can be performed using an exemplary detection apparatus or assay chamber of the invention device. For example, this procedure can be utilized in a sandwich capture assay, in which the RLS gold particles are directly conjugated to a detector antibody.

FIG. 17 shows a flow chart illustrating an exemplary program logic for data query and/or results display. It illustrates a logic path for real-time monitoring of the assay development, and possible immediate notification of results, e.g., positive results.

FIG. 18 shows an example of a subroutine for blob inclusion/rejection. This flow chart illustrates, inter alia, an exemplary mathematical algorithm for calculating a results or positive reactions. It shows that accommodation can be made to eliminate outlier values.

FIG. 19 shows an example of a blob mean pixel intensity acquisition. This shows an example of a logic diagram for analysis of array spots (“blobs”). The flow chart shows examples of how the positive and negative concurrent controls can be used, e.g., to establish an assay range.

FIG. 20 shows an example of a reagent reservoir delivery unit with the parts as disassembled in an exploded view.

FIG. 21 shows photographs of agent detection results for the detection of anthrax.

FIG. 22 shows an example of real-time or time point monitoring of an assay of the present invention with increasing time from left to right.

FIG. 23 shows examples of RLS signals from experiments simultaneously detecting B. anthracis Protective Antigen (PA); B. globigii, a simulant for gram-positive bacteria; Staphylococcal enterotoxin B; C. botulinum toxoid A; Y. pestis; and Ricin A chain. Note for each panel, one agent was not included in each assay.

FIG. 24 shows results for the detection of ricin and botulinum toxoid. The top row shows positive results for the two toxins (at each end of the array) and negative results for all other antigen detection spots/sites (B. anthracis Protective Antigen; B. globigii; Staphylococcal enterotoxin B; and Y. pestis). Bottom row shows the positive controls for these two toxins. Positive controls were deposited and dried in the flow cell. The center row represents negative controls.

FIG. 25 shows an agent detection assay comparing movement of the reagents in a microfluidic fashion compared to a static, non-movement type of assay.

### 5. DETAILED DESCRIPTION

### Definitions

The terms “agent” and “analytes” are used interchangeably herein. Both terms refer to a cell, compound, molecule or other item (e.g., in a sample) to be detected using an assay or apparatus described in various embodiments of the invention. Examples include, but are not limited to, a lipid, a polysaccharide, a polypeptide, a nucleic acid, a bacterial cell, a virus, or a fungal cell. An agent can be an organism or a part of an organism. For example, detection of a virus can mean detection of a viral protein or nucleic acid.

The term “antibody fragment” includes fragments or derivatives derived from an antibody molecule, which fragments or derivatives retain all or a portion of the binding function of a whole antibody molecule. Such immunoreactive fragments or derivatives include those which are known to those skilled in the art and include F(ab′)2′ Fab′, Fab, Fv, scFY, Fd′ and Fd fragments. Methods for producing various fragments or derivatives from antibodies are known in the art. Fragments or derivatives of antibodies or any protein can be made from the protein itself (e.g., using protease digestion) or recombinantly, e.g., by expressing a portion of a protein using a portion of a coding region for the protein.

The term “light scattering particles” refers to particles having the ability to scatter light (e.g., light of visible wavelengths). In many instances, this ability to scatter light will result in scattering sufficient enough to be useful as labels in analyte/agent detection assays. For example, such particles include metal or metal-like materials as described herein. It is recognized that all particles will scatter light to a varying amount depending on their composition, size and shape.

By the term “nucleobase” refers to a nucleic acid moiety including, but not limited to: nucleosides (including, but not limited to synthetic or modified nucleosides), a nucleoside comprising a reactive functionality (e.g., free amino group or carboxyl group); nucleotides (including dNTPs, ddNTPs, and a nucleotide comprising a reactive functionality (e.g., free amino group or carboxyl group)); acyclonucleoside triphosphates (see, e.g., U.S. Pat. No. 5,558,991); 3′(2′)-amino-modified nucleosides, 3′(2′)-amino-modified nucleotides, 3′(2′)-thiol-modified nucleosides, 3′(2′)-thiol-modified nucleotides (e.g., see U.S. Pat. No. 5,679,785); alkynylamino-nucleotides (see, e.g., as a chain terminator, U.S. Pat. No. 5,151,507); and nucleoside thiotriphosphates (e.g., see U.S. Pat. No. 5,187,085).

A “situs” (plural=“sites” herein) is a distinct or a delimited area, e.g., on a reactive surface or assay chamber.

### Apparatuses for Agent Detection

Detection apparatuses of the invention allow for detecting an agent or agents of interest. Some detection apparatuses of the invention provide a platform for detecting essentially an unlimited number of agents. Detection apparatuses of the invention can also be used to analyze, detect or monitor binding of molecules. In some embodiments, an apparatus of the invention utilizes a removable assay chamber (e.g., a flow cell). This provides the advantage and convenience of having one apparatus that can be utilized to detect a wide range of agents, by changing the assay chamber and/or the assay reagents.

The present invention provides apparatuses for the detection of various agents. The apparatuses of the invention can be utilized to detect one or more agents and in some embodiments simultaneously. In some embodiments, the apparatus has a removable and/or a disposable assay chamber such as a flow cell. A particular assay chamber can be utilized to detect one agent or multiple agents. Some detector apparatuses of the invention are designed to be easily portable, e.g., able to be carried easily by a person and containing a power supply (e.g., a battery, a generator, etc.). In some embodiments, an apparatus comprises a power cord as an option, e.g., for drawing AC and/or DC power. In some embodiments, a detector apparatus of the invention does not contain a battery. Although these embodiments may limit portability or use in the field, it allows for a smaller apparatus which will be advantageous in certain locations such as a doctor's office or the like.

In some embodiments, some or all of the components are contained and/or transported in a waterproof case, e.g., from Pelican Products, Torrance, Calif. such as Pelican case, model 1400. In some embodiments, some or all of the components are contained and/or transported in a case that is waterproof, e.g., when closed. In some embodiments, an apparatus comprises 1, 2, 3, 4, 5, 6, 7, 8, 9, 10 or more pumps (e.g., peristaltic). In some embodiments, an apparatus comprises between from about 1 to about 20, about 1 to about 10, about 1 to about 5, about 1 to about 3, about 2 to about 5, about 4 to about 6, about 5 to about 10, about 7 to about 10, about 8 to about 12, about 10 to about 15, about 12 to about 15, about 13 to about 17, about 15 to about 20, about 17 to about 20, about 18 to about 22, or about 5 to about 15 pumps.

Some apparatuses of the invention comprise one or more components or characteristics selected from the group consisting of a case (e.g., waterproof), pumps (e.g., peristaltic), a power source (e.g., a battery), a fluidics manifold, a camera, a fiber optic light cable, a fiber optic light pipe, a five watt white LED, a valve manifold, a circuit board (e.g., a circuit board stack), a microfluidics valve, a microfluidics valve bank, a peristaltic pump bank (e.g., 3 pumps, a power converter, a custom printed circuit controller board stack (e.g., four boards), an assay chamber (e.g., a flow cell), an assay chamber clamp handle, a clamp shoe, a mobile element that presses against a flow cell or assay chamber and establishes leak-proof connections, a slot or position for and assay chamber or flow cell, a focusing lens, and a camera body, an AC Line cord connector, a power switch (e.g., AC/Battery/Off), a computer (e.g., a OQO computer), a computer which provides all program control for the detection apparatus, a reagent reservoir delivery unit, device or mechanism, a machine deck panel, an ability to transmit information (e.g., via cell towers, WiFi, telephone lines, cables (e.g., network cables) and/or via satellite) and a GPS receiver. In some embodiments, an apparatus comprises a heater and/or cooler for maintaining an assay reagent(s), a sample(s), and/or the assay at a particular temperatures or temperature ranges.

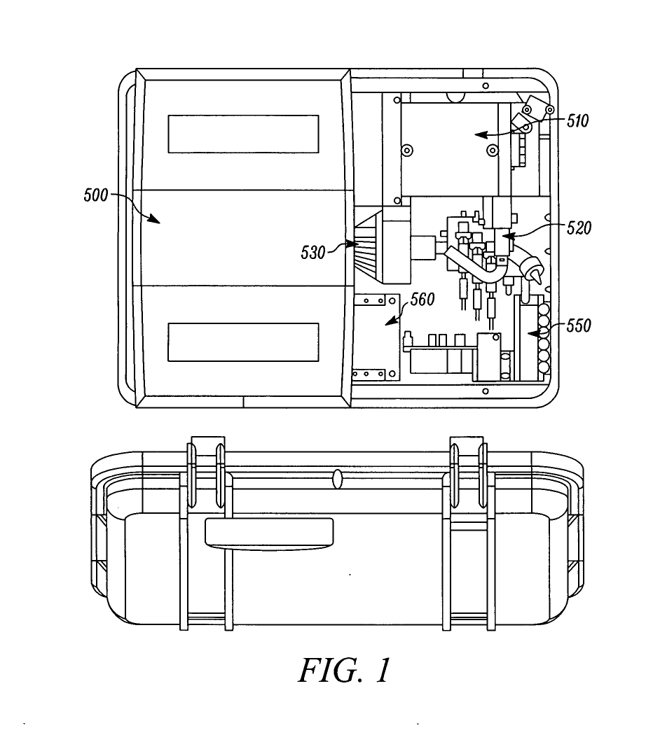

FIGS. 1-6 show various views of one exemplary embodiment of the present invention.

FIG. 1 is a bottom view of an exemplary detection apparatus of the present invention shown with the bottom of the case removed to show some of the internal components including, but not limited to, a battery 500, a camera inspection door 510, a fiber optic light pipe 520, a five watt white LED and a housing 530, a custom valve manifold 550 and a circuit board stack 560.

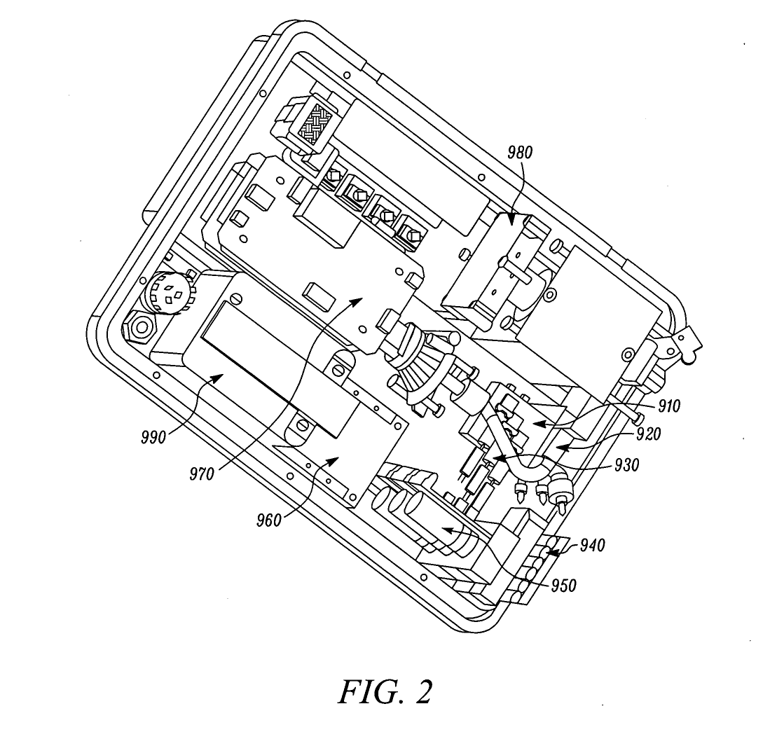

FIG. 2 is a bottom view of an exemplary detection apparatus of the present invention shown with the battery removed to show some of the internal components including, but not limited to, a custom fluidics manifold 910, a fiber optic light cable 920, a microfluidics valve 930, a microfluidics valve bank #2 940, a peristaltic pump bank (3 pumps) 950, an AC/DC power supply 990, a DC/DC power converter 960, a printed circuit controller board stack (four boards) 970, and a complementary metal oxide semiconductor (CMOS) camera 980.

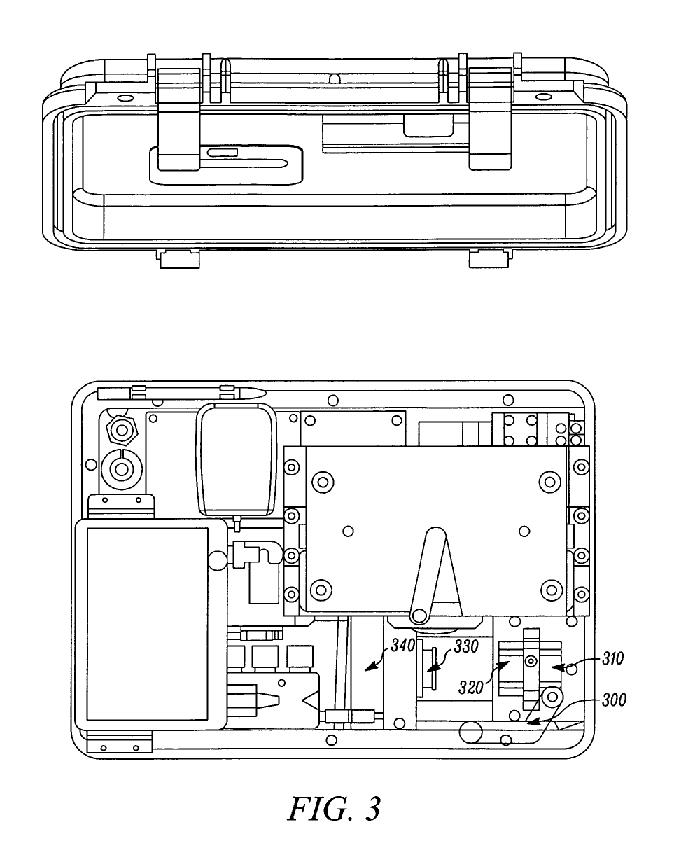

FIG. 3 is a top view of an exemplary detection apparatus of the present invention shown with the lid of the case open to show some of the internal components including, but not limited to, a flow cell clamp handle 300, a clamp shoe, mobile element that presses against flow cell and establishes leak-proof connections 310, a slot for flow cell 320, a focusing lens 330, and a camera body 340.

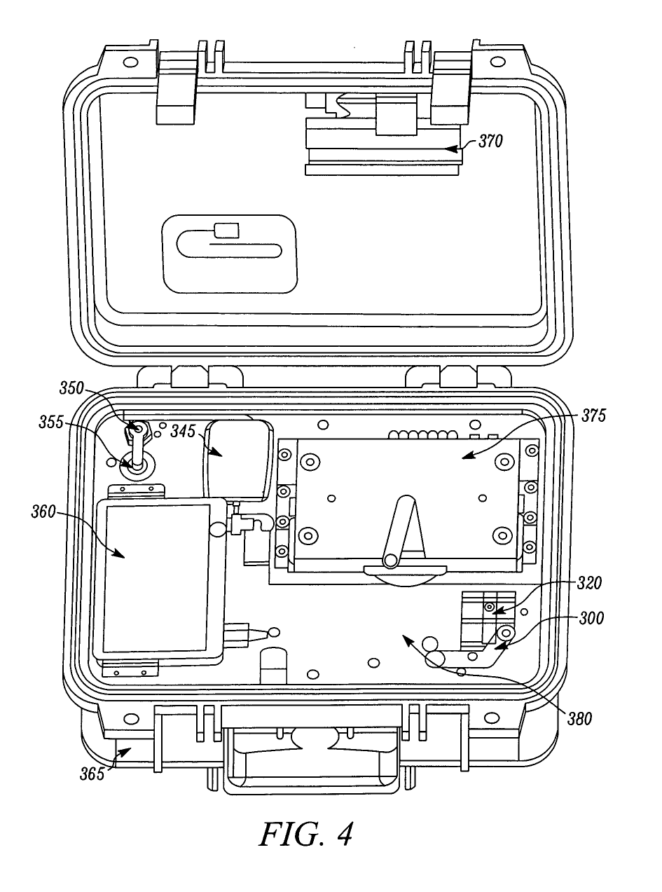

FIG. 4 is a top view of an exemplary detection apparatus of the present invention with the lid of the case open to show some of the internal components including, but not limited to, an AC Line cord connector 350, a power switch (AC/Battery/Off) 355, an OQO computer, which provides all program control for the detection apparatus 360, a Pelican case, model 1400, waterproof, chemical-resistant and lockable 365, a cord keeper unit, space for line cord storage during transport and when using battery 370, a reagent reservoir delivery unit 375, a slot to receive a flow cell 320, a flow cell clamp handle 300, a machine deck panel 380, and a GPS receiver 345. In some embodiments, a GPS receiver senses geographical location and reports to a computer, e.g., for inclusion in reports.

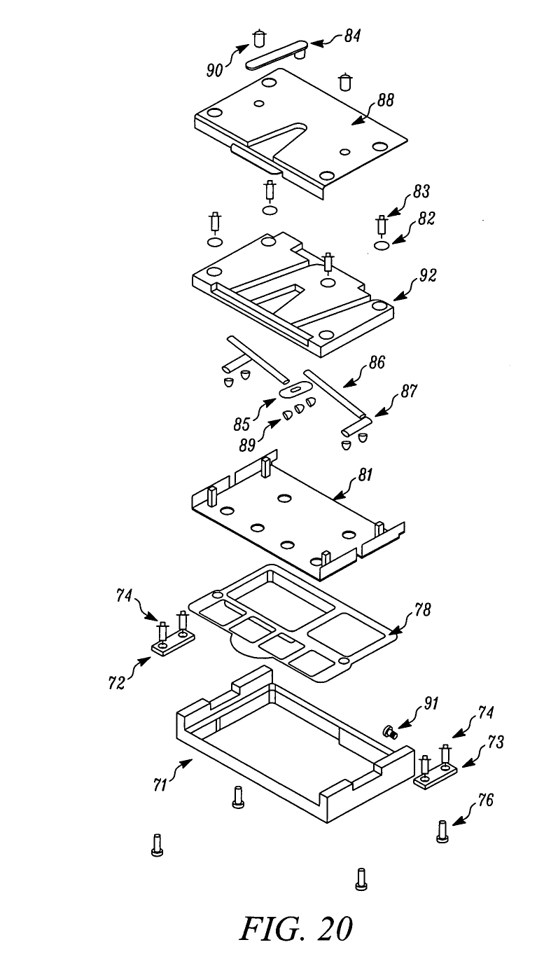

In some embodiments, a reagent reservoir delivery unit 375 holds a disposable cartridge or reagent pack with liquid reagents for an assay. A reagent reservoir delivery unit 375 can have a hinged top and contains hidden hollow pins (e.g., 6) which access the various reservoir compartments by piercing a cover (e.g., a foil cover) on the reservoir pack. In some embodiments, a flow cell or assay chamber is inserted, e.g., into a slot such as 320 in FIG. 4 and a clamping device establishes at least one leak-proof connection (e.g., 6 connections) to a microfluidic channel(s) in the flow cell or assay chamber. FIG. 20 shows an example of a reagent reservoir delivery unit with the parts as disassembled in an exploded view. The exemplary reagent reservoir delivery unit of FIG. 20 comprises the items/components listed in Table 1.

TABLE 1

| ITEM # | PART NUMBER | DESCRIPTION | QTY. |
|---|---|---|---|
| 71 | 100050 X2 Bottom Clamshell | Bottom Clamshell X2 | 1 |
| 72 | 100052 X2 Striker Plate Right | Striker Plate Right X2 | 1 |
| 73 | 100051 X2 Striker Plate Left | Striker Plate Left X2 | 1 |
| 74 | Screw Low Head Socket Cap Screw 8 × 32 | McMaster #92220A153 | 4 |
| 75 | Mounting Plate | Not shown | 1 |
| 76 | Screw 8-32 0.5 | McMaster #91400A194 | 4 |
| 78 | Reagent/Cleaning Pack | Blister Pack | 1 |
| 81 | 100053 X2 Needle Guide Plate | Needle Guide Plate | 1 |
| 82 | 100054 X2 Cover Washer | Cover Washer X2 | 4 |
| 83 | Screw 4 × 40 0.6875 | McMaster #91400A114 | 4 |
| 84 | 100056 X3 Handle Latch | Handle Latch X3 | 1 |
| 85 | 100057 X2 Handle Link | Handle Link X2 | 1 |
| 86 | 100058 X2 Latch Pall. Right | Latch Pall X2 | 2 |
| 87 | 100059 X2 Latch Keeper | Latch Keeper X2 | 2 |
| 88 | 100060 X2 Cover Needle Plate | Cover Needle Plate X2 | 1 |
| 89 | Screw Flat 2-56 × 0.125 | McMaster #96877A110 | 7 |
| 90 | Screw Flat 4 × 40 0.375 McMaster-96877 | McMaster #96877A209 | 2 |
| 91 | Hinge Shoulder Screw 0.125-Dia | McMaster #93996A516 | 2 |
| 92 | 100055 X4 Top Clamshell | Top Clamshell X4 | 1 |

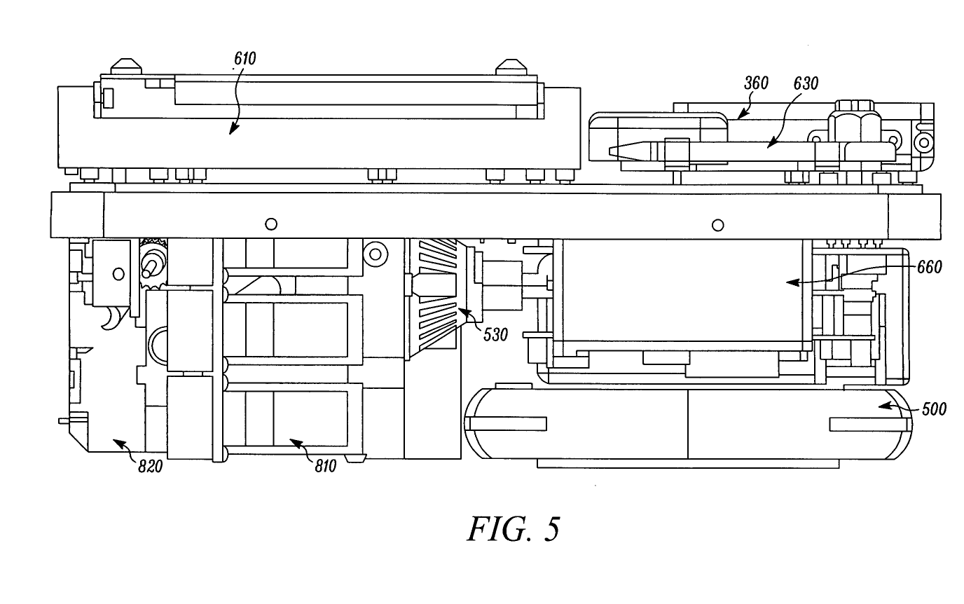

FIG. 5 is a side view of a detection apparatus of the present invention shown without the case to show some of the internal components including, but not limited to, a base of a reagent reservoir delivery unit 610, an OQO computer 360, a tablet PC (personal computer) stylus 630, a power supply unit 660, a battery which may be rechargeable 500, a five watt white LED and housing 530, peristaltic pumps 810, and valve/plumbing area 820.

In some embodiments, a reagent reservoir delivery unit provides a receptacle for a reagent pack(s) (e.g., a “single-use” reagent and/or cleaning pack). In some embodiments, a reagent reservoir delivery unit comprises a top plate mechanism that pivots and pierces the lidding stock of a reagent pack. In some embodiments, a top plate mechanism locks in a closed position. In some embodiments, top plate incorporates a fluidic manifold to deliver the reagents to and from an assay chamber.

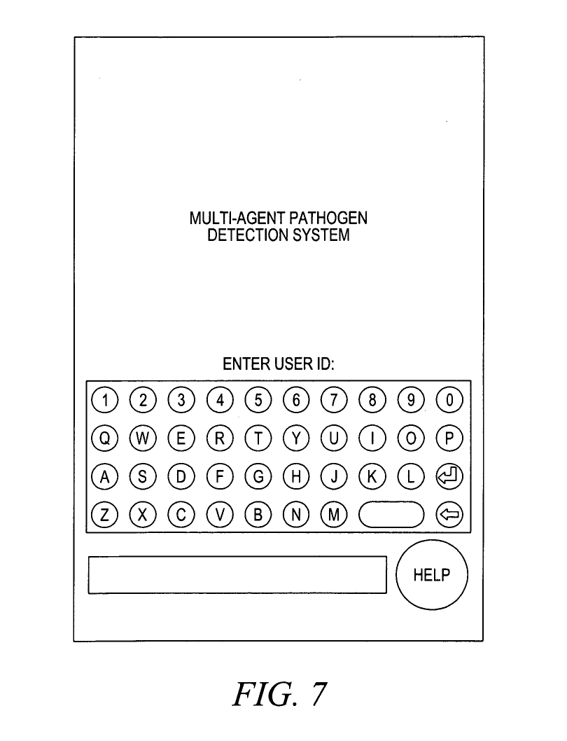

FIG. 7 is an example of a GUI of the present invention. This GUI shows (a) two banners 900 and 910, which can display any information, e.g., a manufacturer's name, a name for an assay, a name for the detection apparatus such as a trade name and/or instructions; (b) a keyboard for inputting information 920; (c) a help button that will bring up general or specific help 920; and (d) a window to show information as typed-in 930.

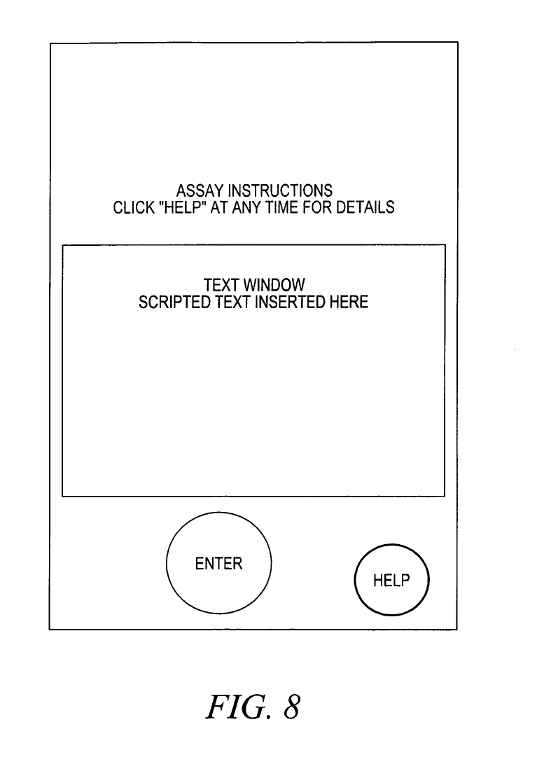

FIG. 8 is another example of a GUI of the present invention. This GUI shows (a) two banners 970 and 940, which can display any information, e.g., a manufacturer's name, a name for an assay, a name for the detection apparatus such as a trade name and instructions; (b) a window to display information 950, e.g., status of an assay, results of an assay, detailed information about any agent such as precautions and/or treatments, and instructions for a user such as when to insert a sample or any error messages; (c) a help button that will bring up general or specific help 920; and an enter button, e.g., used to confirm that a particular instruction displayed in 950 has been completed.

In some embodiments, a GUI is utilized wherein “windows” are shown to the user with explicit instructions for each step in the assay, and optionally with a countdown timer, e.g., to show assay progress. For example, a display in the text window in FIG. 8 might be “Rotate Flow Cell Clamp Crank Handle to Full Counter-clockwise Position. Click Enter”

In some aspects of the invention, a detection apparatus is designed to be portable. In some embodiments, the apparatus is powered by a portable power source (e.g., a solar power, a generator or a battery, such as AA batteries. Batteries used with the invention may be rechargeable. In some embodiments, a battery is a Nickel-Metal Hydride (NiMH) battery, such as an Energy+Powebase-Jr™ produced by Fedco Electronics, Inc (Fond Du Lac, Wis.). In some embodiments, a battery is a Lithium battery such as from Ultralife Batteries, Inc (Newark, N.Y.). In some embodiments, a detection apparatus is powered by plugging into a power source. In some embodiments, the apparatus is capable of being powered by an accompanying power source or by plugging into a power source (e.g., an electrical outlet). In some of these embodiments, the accompanying power source (e.g., a battery) charges while plugged into a power source.

Some detector apparatuses of the present invention include a global positioning system (GPS). In some embodiments, a GPS is USB based. In some embodiments, a GPS receiver is an “Earthmate GPS” Model LT-20 made by DeLorme (Yarmouth, Me.); a model BU-353 from US Global Sat, Inc. (City of Industry, Calif.) or a unit from Garmin (Olathe, Kans.). In some embodiments, a GPS unit is linked to a computer for communication with the computer.

Some detector apparatuses of the present invention include a computer. In some embodiments, a computer is a Model 01+ (touch screen interface running Windows XP Tablet software) from OQO (San Francisco, Calif.). In some embodiments, a computer is a PDA (e.g., from Dell (Round Rock, Tex.) or AMREL (El Monte, Calif.)) or a Recon PDA from Geneq, Inc. (Montreal, Canada). In some embodiments, a computer is a full size tablet PC (e.g., from Itronix (Spokane Valley, Wash.) or Xplore Technologies (Austin, Tex.)). In some embodiments, a computer is linked to a touch-screen.

In some embodiments, some or all of the operation of the apparatus is controlled by the computer and/or touch-screen prompts; a sample chamber (e.g., a flow cell) is manually inserted appropriately into the detection apparatus; and/or a sample is manually introduced into the assay. In some embodiments, a user is prompted by a computer or touch-screen for some or all steps in an assay procedure. A computer can serve a multitude of functions and combination of functions. In some embodiments, a computer is linked to a detection means, e.g., a CCD camera (e.g., a SenSys CCD, Photometrics, Tucson, Ariz.) or photomultiplier tube, so that it can receive data. In some embodiments, a camera has a resolution of 500×500 pixels or greater. The computer can be used, e.g., with a particular software program, to analyze data and display whether a sample is determined to be positive for a particular agent(s) and/or to determine the quantity and/or concentration of an agent in a sample. The computer can also be linked to a GPS, e.g., the computer can record the location that each sample is collected and/or assayed. Additionally, a computer can record the date and/or time of collection and/or analysis of a sample. Thus, the invention includes methods for tracking information (e.g., date, time, sample size, sample characteristics, etc.). The present invention also provides methods for measuring, tracking and analyzing agents over an area.

In some embodiments, a detection apparatus is capable of transmitting data, e.g., wireless or using wired communication (e.g., over the internet/world-wide-web). This can allow, for example, for communicating the results of an assay to another location. In some embodiments, this communication is performed automatically with each sample analysis. Communication can be any means, e.g., via cell phone towers/networks, WiFi, satellite communications, phone lines, networks, etc. Thus, the invention provides methods for rapid generation and compilation of data over a geographic area. A geographic area can be a room, a building, a neighborhood, a campus, a city, a country, a continent, a battlefield, a body of water, and a farm field. In some embodiments, a geographic area is between from about 10 to about 5000, from about 1000 to about 5000, from about 2000 to about 5000, from about 3000 to about 5000, from about 4000 to about 5000, from about 10 to about 100, from about 100 to about 200, from about 200 to about 400, from about 400 to about 600, from about 600 to about 800, of from about 800 to about 1000 square ft. In some embodiments, a geographic area is between from about 1 to about 100,000, from about 100 to about 100,000, from about 1,000 to about 100,000, from about 10,000 to about 100,000, from about 1 to about 10, from about 10 to about 100, from about 100 to about 1,000 or from about 1,000 to about 10,000 acres. In some embodiments, a geographic area is between from about 10 to about 5000, from about 100 to about 5000, from about 1000 to about 5000, from about 2000 to about 5000, from about 3000 to about 5000, from about 4000 to about 5000, from about 10 to about 100, from about 100 to about 200, from about 200 to about 400, from about 400 to about 600, from about 600 to about 800, or of from about 800 to about 1000 square miles.

In some embodiments, results from an assay are received, generated, analyzed and/or compiled within a time period of between from about 10 minutes to about 24 hours, from about 10 minutes to about 16 hours, from about 10 minutes to about 12 hours, from about 10 minutes to about 6 hours, from about 10 minutes to about 3 hours, from about 10 minutes to about 1 hour, from about 10 minutes to about 40 minutes, from about 10 minutes to about 20 minutes, from about 10 minutes to about 15 minutes, from about 15 minutes to about 20 minutes, from about 13 minutes to about 18 minutes, from about 20 minutes to about 40 minutes, from about 40 minutes to about 60 minutes, from about 1 hour to about 2 hours, from about 2 hours to about 4 hours, from about 4 hours to about 6 hours, from about 6 hours to about 12 hours, from about 12 hours to about 16 hours or from about 16 hours to about 24 hours. These times may be from the start of an assay (e.g., input of a sample) to 1) a result(s), 2) to a transmittal of a result(s), or 3) compilation of result(s).

Some embodiments of the invention employ two or more apparatuses with data assembled and/or compiled from at least two of the two or more apparatuses.

Some detector apparatuses of the present invention include a device, position, or reservoirs for reagents for the assay. Reagent reservoirs can be realized by any number of methods or means. In some embodiments, one or more reagent reservoirs can be reservoirs built-in to an apparatus, e.g., that a user fills or replaces as necessary or depending on the assay to be performed. In some embodiments, one or more reagent reservoirs are replaceable or interchangeable. For example a reagent reservoir can be a tube, a blister of a blister pack, or any container or chamber that can hold and is compatible with the reagent. By compatible means that when the reagent(s) and reagent reservoir are in contact, they do not react in a way to inhibit the intended purpose of the reagent(s) or reagent reservoir.

In some embodiments of the invention, a detection apparatus comprises a device that is fitted with a reagent pack referred to herein as a reagent reservoir delivery unit. In some of these embodiments, a reagent pack is placed in a reagent reservoir delivery unit and reagents are automatically withdrawn from the reagent pack as necessary for a particular assay being performed. Reagent packs of the present invention find use not only with the detection apparatuses and methods of the invention, but with any method or apparatus using a reagent or more than one reagent. An exemplary reagent pack or cleaning pack is shown in FIG. 20 as 78.

A reagent pack can be of any material that is compatible with the contained reagents. In some embodiments, reservoirs of a reagent and/or cleaning pack are made of a plastic, glass, polypropylene, polyethylene or polystyrene. In some embodiments, at least one reservoir of a reagent or cleaning pack is clear, colored or opaque. In some embodiments, the reservoirs of a reagent pack are formed via a vacuum thermoforming process, via machining and/or via injection molding. In some embodiments, a reagent or cleaning pack comprises at least one reservoir and lidding. In some embodiments, the side of the lidding facing at least one reservoir is hydrophobic. In some embodiments, lidding is attached to a reservoir structure via a heat seal or thermal bond. In some embodiments, a reservoir structure is a blister reservoir or a blister tray.

In some embodiments, there are various chambers for various reagents of the assay. In some embodiments, one or more replaceable or interchangeable reagent reservoirs are physically attached, e.g., a strip of tubes or a blister pack to create a pack. This can allow the changing of more than one assay reagent at a time. It also reduces errors by linking a set of reagents for a particular assay. Thus, the present invention provides methods for reducing errors by providing reagent packs as described herein, for example, by providing a portion or all necessary reagents for an assay. Thus, reducing the chances mismatching the reagents. Also, the present invention provides methods for providing reagents, e.g., to an apparatus. The apparatus is not necessarily a detection apparatus as described herein.

In some embodiments, a reagent pack comprises a bottom portion comprising at least one reservoir. In some embodiments, a reagent pack comprises a top portion covering and or sealing the top of the reservoir(s). In some embodiments, the top portion is made of a material that seals in the reagent(s), but is able to be pierced by a port for removing the reagent(s). In some embodiments, a reagent pack is a blister pack or of similar design.

In some embodiments, a reservoir in a reagent pack or blister pack contains an assay buffer (e.g., wash buffer, a blocking buffer and/or a diluent buffer). In some embodiments, a reagent pack or blister pack comprises a label (e.g., a computer readable label such as a bar code) identifying the reagent pack. In some embodiments, a label on a reagent pack is read by a detection apparatus. In some embodiments, a detection apparatus, reads a label on a reagent pack and verifies that the reagent pack is compatible with the desired assay to be run. In some embodiments, a detection apparatus, reads a label on a reagent pack and reads a label on an assay chamber and determines if they are intended for the desired assay to be performed. In some embodiments, a detection apparatus will display a message if an inserted reagent pack and/or an inserted assay chamber are not intended for use with each other or the assay to be performed. This message can be displayed, for example, via a GUI. In some embodiments, if a reagent pack and an assay chamber are inserted, which are not compatible (e.g., do not match for the desired assay to be performed) an audible alarm is sounded and/or a visible alarm is sounded.

In some embodiments, a reagent pack only provides reagents specific for a certain assay or assays. For example, some detection apparatuses of the invention have the advantage of using the same hardware, except for an interchangeable assay chamber and/or assay reagents to analyze a multitude of agents. In this way, one apparatus can be used to test a multitude of agents by just switching out the assay chamber(s) or flow cell(s) and in some cases utilizing some different reagents. Along the same lines, some of the assay reagents may be the same (e.g. wash buffer) between different assays. Therefore, in some embodiments, a reagent pack may be comprised of, or consist essentially of, reagents that are common to more than one assay that is performed with different assay chambers. In some embodiments, a detection apparatus has built in reservoirs for common reagents and/or non-common reagents. In some embodiments, a reagent pack comprises or consists essentially of assay reagents that are specific for an assay chamber or flow cell. In some embodiments, two reagent packs are utilized for an assay or assay chamber. For example, one reagent pack is specific for an assay chamber(s) (e.g., a flow cell) or method to be performed and the second one is generic, e.g., generic for the assay format or assay type. For example, many sandwich type assays can utilize the same wash buffers, diluents and in some cases the same labeled binding molecules (e.g., a labeled anti-biotin antibody). In some embodiments, a reagent(s) in a reagent pack is at a working dilution. In some embodiments, a reagent(s) in a reagent pack is concentrated. In some of these embodiments, the concentrated reagent(s) is diluted manually (e.g., by a user) or the concentrated reagent(s) is diluted by the apparatus (e.g., automatically).

In some embodiments, a reservoir in a reagent pack or blister pack is used for waste collection. This reservoir is sometimes referred to as a waste reservoir. In some embodiments, the apparatus comprises a waste reservoir that is separate from a reagent pack. Typically a waste reservoir is of sufficient volume to hold all or a portion of the wasted or spent reagents. In some embodiments, assay reagents and/or at least a portion of the test sample, are deposited (e.g., automatically) into a waste reservoir(s). In some embodiments, a waste chamber comprises a decontamination, sanitizing, and/or deactivating compound(s). In some embodiments, this compound is an alcohol, a sodium hypochlorite, a quaternary ammonium compound or material similar to these. In some embodiments, decontamination, sanitizing, and/or deactivating compound(s) is selected from the group consisting of a chlorine containing disinfectant (e.g., sodium hypochlorite); a stabilized chlorine dioxide, a phenol, a chlorhexidine gluconate, a quaternary ammonium compound, a glutaraldehyde, an alcohol, an iodine containing compound, a pine oil or a mercury compound. Some decontamination, sanitizing, and/or deactivating compound(s) that could be used are commercially available including, but not limited to, Wescodyne® (Steris, Mentor, Ohio) or Cidex® (Advanced Sterilization Products, Irvine, Calif.). In some embodiments, the waste chamber is exposed to conditions that decontaminate, sanitize, or deactivate agents. These conditions include, but are not limited to, irradiation, ultraviolet irradiation, heating, cooling (e.g., freezing), gamma irradiation, or combinations thereof. Additionally, the invention contemplates the combination of any of the conditions with any decontamination, sanitizing, and/or deactivating compound(s). In some embodiments, a compound that decontaminates, sanitizes, or deactivates an agent is provided in a chamber separate from a waste chamber and the decontaminating agent is transported (e.g., via a pump) to a waste reservoir. In some embodiments, a compartment of a reagent pack contains a binding molecule (e.g., a labeled antibody (e.g., with gold particles)), and optionally a decontamination solution.

In some embodiments, a waste reservoir contains a material (e.g., a powder) that immobilizes waste fluids and optionally contains a decontaminating compound(s). For example, the material could be a gel-forming material such as a gel forming powder or a gel that can absorb reagent waste. A gel-forming material comprises a compound(s) that forms a gel, before, upon or after contact with a sample, sample waste, an assay reagent, and/or an assay reagent waste. In some embodiments, a waste reservoir contains an absorbent material, such as a sponge, that immobilizes waste fluids and optionally contains a decontaminating compound(s). These embodiments provide an advantage that waste or spent reagents are essentially converted to a gel and/or solid and optionally decontaminated, thus reducing or eliminating the spilling or leaking of reagents. Thus, the present invention provides methods for reducing hazards associated with an agent and/or assay reagents. In some embodiments, a reservoir contains a decontamination reagent which is pumped through a detection apparatus or assay chamber, typically at the end of an assay and/or after sample introduction. Thus, the invention also provides methods for the collection and/or decontamination of reagents and/or samples.

Thus, the present invention provides methods for the safe handling of waste and methods. The present invention also provides compositions and methods for rendering a reagent or waste reagent less or non-hazardous.

A reagent pack of the invention is not limited for use in detection assay of the invention, but can be used in other apparatuses, devices and methods using one or more reagents. These embodiments may include a reservoir device (e.g., as described herein) that holds a reagent pack of the invention.

A blister pack or reagent pack of the present invention comprises 1, 2, 3, 4, 5, 6, 7, 8, 9, 10, 11, 12, 13, 14, 15, 16, 17, 18, 19, 20, 21, 22, 23, 24, 25, or more compartments or reservoirs. In some embodiments, a blister pack or reagent pack of the present invention comprises between from about 1 to about 30, from about 1 to about 10, from about 1 to about 20, from about 20 to about 30, from about 10 to about 30, from about 1 to about 4, from about 3 to about 6, from about 5 to about 10, from about 5 to about 10, from about 7 to about 12, from about 10 to about 15, from about 12 to about 17, from about 15 to about 20, from about 17 to about 22, from about 20 to about 25, from about 22 to about 27, from about 25 to about 30 or from about 27 to about 30 compartments or reservoirs.

In some embodiments, an individual compartment(s) of a blister pack or reagent pack contains a volume of reagents or is capable of holding a volume between from about 1 μl to about 100 ml, from about 1 μl to about 1 ml, from about 10 μl to about 1 ml, from about 100 μl to about 1 ml, from about 500 μl to about 1 ml, from about 1 μl to about 500 μl from about 1 ml to about 10 ml, from about 10 ml to about 100 ml, from about 1 μl to about 10 μl, about 10 μl to about 50 μl, about 50 μl to about 100 μl, about 100 μl to about 200 μl, about 200 μl to about 300 μl, about 300 μl to about 400 μl, about 400 μl to about 500 μμl to about 600 μl, about 600 μl to about 700 μl, about 700 μl to about 800 μl, about 800 μl to about 900 μl, about 900 μl to about 1.0 ml, about 1.0 ml to about 1.5 ml, about 1.5 ml to about 2.0 ml, about 2.0 ml to about 2.5 ml, about 2.5 ml to about 3.0 ml, about 3.0 ml to about 3.5 ml, about 3.5 ml to about 4.0 ml, about 4.0 ml to about 4.5 ml, about 4.5 ml to about 5.0 ml, about 5.0 ml to about 5.5 ml, about 5.5 ml to about 6.0 ml, about 6.0 ml to about 6.5 ml, about 6.5 ml to about 7.0 ml, about 7.0 ml to about 7.5 ml, about 7.5 ml to about 8.0 ml, about 8.0 ml to about 8.5 ml, about 8.5 ml to about 9.0 ml, about 9.0 ml to about 9.5 ml, about 9.5 ml to about 10 ml, about 10 ml to about 15 ml, about 15 ml to about 20 ml, about 20 ml to about 30 ml, about 30 ml to about 50 ml, about 50 ml to about 100 ml, about 100 ml to about 200 ml, or more.

The present invention also provides a device that is capable of being fitted with a reagent pack, referred to herein as a reagent reservoir delivery unit. In some embodiments, a reagent reservoir delivery unit comprises one or more components. In some embodiments, one component is a hardware “shell”. A hardware shell may include a hinged top of the shell that opens, and allows for the insertion of a reagent pack (e.g., a disposable fluidic blister pack). In some embodiments, reagent reservoir delivery unit comprises “needles”, pins, tubes or ports that withdraw reagents from a reagent pack. In some embodiments, the lid has a spring-mounted plate that conceals pins, needles, or ports that pierce a reagent pack or reagent blister pack, and allow the fluids to be pumped into the system. In some embodiments, the needles, tubes or ports are not exposed or accessible when the device is “open”, but when closed, the pins, needles, tubes or ports insert into various reagents, e.g., by piercing a reagent blister pack. In some embodiments, when the top or lid of a reagent reservoir delivery unit is opened to insert or remove a reagent pack, the pins, needles or ports again are refracted and/or hidden by the top plate. Thus, the present invention provides a method for packaging reagents. The invention also provides a method for delivering reagents to, for example, a device, apparatus, assay chamber, and/or flow cell. An exemplary reagent reservoir delivery unit is shown in FIG. 20 and FIG. 4 as 375.

Using FIG. 20 as an example, the Top Clamshell 92 is comprised of tubes or ports for withdrawing reagents from the reservoirs. In some embodiments, the tubes are made from hypodermic tubing. The tubes may be made from essentially any material that is compatible with the reagents and the insertion method used to insert into a reagent pack. In some embodiments, a tube comprises stainless steel. In some embodiments, tubing such as hypodermic tubing is pressed into a top clamshell or similar device. In some embodiments, it is interference press fit. In some embodiments, tubes are attached without adhesive, glue or gaskets to a top clamshell or similar device. In some embodiments, tubes are attached to a top clamshell or similar device to form a continuous fluidic path. In some embodiments, tubes are blunt or pointed such as a needle. In some embodiments, the tubes are polished before and/or after attachment.

In some embodiments, a reagent pack comprises a multichamber bag. In some embodiments, a multichamber bag is connected to an assay chamber and/or detection apparatus via a multiport connector such as those commercially available. In some embodiments, a multichamber bag is formed from 2 sheets of material such as plastic. Examples of multichamber bags are described for example in U.S. Pat. No. 5,207,509.

In some embodiments, reagents are delivered to an assay chamber (e.g., a flow cell) using pumps and/or a pump system. In some embodiments, a pump that can be utilized with the present invention is a peristaltic pump, a micro-peristaltic pump, a solenoid pump, a miniature solenoid pump, or combinations thereof. In some embodiments, a pump is a positive (e.g., self-priming) displacement pump. Positive displacement pumps include, but are not limited to, a Micro gear pump (e.g., from MicroPump, Vancouver, Wash.), a piston-pump (e.g., from MicroPump), a peristaltic pump (e.g., from Instech, Plymouth Meeting, Pa.), a solenoid pump (e.g., from Lee Co., Westbrook, Conn.), a DC motor drive diaphragm pump (e.g., from Smart Products, Morgan Hill, Calif. or KNF, Trenton, N.J.), a piezo-actuated micro diaphragm pumps (e.g., from ThinXXS, Germany), or a Syringe pump (e.g., from Kloehn Ltd. (Las Vegas, Nev.), Hamilton (Reno, Nev.), or Harvard Apparatus (Holliston, Mass.)). In some embodiments, a pump is a non-priming pump such as a Centrifugal pump (e.g., from MicroPump or 3M Sams). In some embodiments, a peristaltic pump is Model P625/66.133 from Instech (Plymouth Meeting, Pa.).

In some embodiments of the invention, the sample chamber or device includes a computer readable label such as a bar code. In some embodiments, an LED system is utilized to light a bar code. In some embodiments, a bar code is read by a camera and recorded by a computer.

In some embodiments, a sample chamber, a reactive surface or a chamber in which the assay is performed is comprised of a binding array (e.g., a printed array), plastic machined superstructures, and a custom laser-cut gasket.

In some embodiments, a detection apparatus of the invention comprises a heater for maintaining assay reagents and/or the assay at a particular temperatures or temperature ranges throughout an assay procedure or at various times during the procedure. A heater can also be used to maintain reagents and/or a component(s) of an apparatus above a certain temperature, e.g., above temperatures that would cause the reagents to freeze or above temperatures that may be harmful to a component(s) of a detection apparatus of the invention. In some embodiments of the invention, a detection apparatus comprises a cooling device which cools reagents and/or a component(s) of a detection apparatus. In some embodiments, reagents are cooled until their use in an assay, e.g., at 4° C. In some embodiments, the components of an assay are cooled during at least one assay procedure to prevent the assay reagents from reaching detrimental temperatures, e.g., in extreme heat conditions such as can be found is a desert. In some embodiments, a detection apparatus utilizes a heating and/or cooling device for storing samples at controlled temperatures. In some embodiments, a detection apparatus of the invention does not comprise a heating device. In some embodiments, a detection apparatus of the invention does not comprise a cooling device. In some embodiments, fluids entering an assay are at about 37° C. In some embodiments, fluids entering an assay, are between from about 30° C. to 42° C., from about 32° C. to 39° C., from about 33° C. to 39° C., from about 34° C. to 39° C., from about 34° C. to 39° C., from to 39° C., from about 35° C. to 38° C., from about 35° C. to 37° C., from about 36° C. to 38° C., or from about 30° C. to 38° C. In some embodiments, reagents are at ambient temperature during assay procedures.

In some instances, the combined weight of components for a detection apparatus of the invention can be important. In some embodiments, an apparatus of the invention is designed as a readily portable apparatus, e.g., able to be carried by an average person. In some embodiments, the combined weight of components for a detection apparatus is less than 1, less than 2, less than 3, less than 4, less than 5, less than 6, less than 7, less than 8, less than 9, less than 10, less 11, less than 12, less than 13, less than 14, less than 15, less than 16, less than 17, less than 18, less than 19, less than 20, less than 21, less than 22, less than 23, less than 24, or less than 25 kilograms. In some embodiments, the combined weight of components for a detection apparatus is between from about 1 to about 25, from about 5 to about 15, from about 5 to about 10, from about 5 to about 20, from about 5 to about 25, from about 10 to about 25, from about 10 to about 15, or from about 15 to about 25 kilograms. In some embodiments, a detection apparatus is of a size that can be transported by an average person. In some embodiments, the size of the apparatus has a total volume of between from about 10 cm³ to about 1.5 m³, about 10 cm³ to about 1 m³, about 10 cm³ to about 50 cm³, about 10 cm³ to about 25 cm³, about 50 cm³ to about 100 cm³, about 75 cm³ to about 100 cm³, about 25 cm³ to about 75 cm³, about 35 cm³ to about 55 cm³, about 10 cm³ to about 10 cm³, about 30 cm³ to about 100 cm³, about 30 cm³ to about 50 cm³, about 50 cm³ to about 70 cm³, about 70 cm³ to about 90 cm³ or about 90 cm³ to about 100 cm³.

An apparatus of the invention can comprise a port that allows automatic, and in some cases, continual collection and analysis of samples, e.g., samples are collected at predetermined time periods. Thus, the invention provides apparatuses and methods for automatic continuous collection and monitoring of samples. For example, a detection apparatus can be comprised of components that allow automatic sampling such as filters for air samples or a tube or port for continuous monitoring of water samples. In other words, a detection apparatus may be equipped with a means for automatic sampling and components to automatically process and analyze a sample. For example, a sample can be automatically acquired and assayed utilizing a detection apparatus of the invention. In some embodiments, a sample chamber is automatically replaced with another assay chamber, e.g., for the next sample. In some embodiments, an assay chamber has more than one channels or chamber for sample analysis. In this embodiment, different assay chamber can be utilized to analyze different samples that are automatically or manually acquired at different time points. For example, an assay chamber of the invention may comprise 6 channels such as 2 sets of 3 channels, wherein each set is comprised of one positive control channel, one negative control channel and one channel for sample analysis. Then each set of 3 channels can be used to analyze different samples, for example for different time points. In some embodiments, assays are performed at the same time on different samples. For example, two or more samples are collected at different time points, but they are analyzed at the same time. In other embodiments, two or more samples are collected at different times and each respective assay is run essentially immediately following sampling. In some embodiments, the assays are run consecutively for each sample. In some embodiments, the assays overlap in time. For example, a second assay is started in the same assay chamber for a second time point prior to the completion of the assay for the first sample, for example in different channels of the same assay chamber or flow cell. This can continue based on the number of channels in an assay chamber or flow cell.

In some embodiments, a fluidics pump(s) circulates wash fluid from a reservoir container through the microfluidic chambers in the flow cell. In some embodiments, this serves to pre-wet a fluid path and can be designed to assure smooth flow.

In some embodiments, a user is prompted by a computer (e.g., a Tablet PC, desktop or laptop) to inject sample and optionally the volume of sample to be injected (e.g., with a syringe into a sample port) is prompted. In some embodiments, excess sample is shuttled to a waste reservoir. In some embodiments, a user/operator then uses the touch-screen to indicate a sample has been injected. Optionally, a sample syringe is removed and discarded, e.g., prior to or during the performance of the rest of the assay procedure. In some embodiments, a check valve prevents back-flow or leakage. In some embodiments, at the same time as sample injection or after indicating the sample has been injected, positive and negative control samples are rehydrated and circulated through independent micro channels in the chamber. In some embodiments, after sample injection and until readout, no user steps are required because the rest of the assay is automated and controlled by a computer.

In some embodiments, a positive and or negative result/hit can result in an audible and/or visible alarm. In some embodiments, if a sample(s) contains an agent(s) with a concentration or level exceeding or below a predetermined value an audible and/or visible alarm is activated. In some embodiments, an audible alarm is sounded only if a positive signal is detected. In some embodiments, different sounds are produced based on a positive or negative result.

In some embodiments, a sample is circulated through a loop, passing repeatedly over a capture binding molecule (e.g., an antibody) in (e.g., bound to a surface of) a test chamber or assay chamber. This recirculation step can increase the speed and efficiency of the capture reaction, which would typically be diffusion-driven. In some embodiments, at least one assay reagent is circulated through a loop, passing repeatedly over the capture binding molecule, e.g., that is bound to a surface of a test chamber. In some embodiments, at least one assay reagent (e.g., a sample) is incubated without recirculation or as static. In some embodiments, recirculation comprises pulsing the movement of the reagent. In some embodiments, movement of a reagent in relation to a capture binding molecule is performed without a loop, e.g., moved in one direction and then in the opposite direction without looping. In some embodiments, loop recirculation involves circulation in one direction of the loop followed by recirculation in another or opposite direction, e.g., pulsing.

Pulsing involves movement of a reagent such as in relation to a capture binding molecule, for a period of time followed by 1) no movement or no induced movement or 2) movement in another direction. In some embodiments, pulsing comprises movement of a reagent (e.g., a sample) followed by a period of static state (e.g., little or no movement), optionally followed by another cycle of the same or different pulsing. In some embodiments, a pulsing cycle comprises a movement step and or a static step for a period of time between from about 0.1 seconds to about 1 hour, about 0.1 seconds to about 30 minutes, about 0.1 seconds to about 15 minutes, about 0.1 seconds to about 10 minutes, about 0.1 seconds to about 5 minutes, about 0.1 seconds to about 4 minutes, about 0.1 seconds to about 3 minutes, about 0.1 seconds to about 2 minutes, about 0.1 seconds to about 1 minute, about 0.1 seconds to about 50 seconds, about 0.1 seconds to about 40 seconds, about 0.1 seconds to about 35 seconds, about 0.1 seconds to about 30 seconds, about 0.1 seconds to about 25 seconds, about 0.1 seconds to about 20 seconds, about 0.1 seconds to about 15 seconds, about 0.1 seconds to about 10 seconds, about 0.1 seconds to about 5 seconds, about 0.1 seconds to about 1 second, about 1 second to about 5 seconds, 2.5 seconds to about 7.5 seconds, about 5 second to about 10 seconds, 7.5 seconds to about 12.5 seconds, about 10 second to about 15 seconds, 12.5 seconds to about 17.5 seconds, about 15 second to about 20 seconds, 17.5 seconds to about 22.5 seconds, about 20 second to about 25 seconds, 22.5 seconds to about 27.5 seconds, about 25 second to about 30 seconds, 30 seconds to about 35 seconds, about 35 second to about 40 seconds, 40 seconds to about 45 seconds, about 45 second to about 50 seconds, 50 seconds to about 55 seconds, or about 55 second to about 60 seconds. In some embodiments, a movement step is a longer period of time than a static step. In some embodiments, a movement step is a shorter period of time than a static step. In some embodiments, a movement step is about the same period of time as a static step. In some embodiments, a cycle comprises a movement step in one direction followed by a static step followed by a movement step in another direction. In some embodiments, a cycle comprises a movement step of 6 seconds followed by a lag or non-movement 24 seconds. A non-movement step includes a lag or non-circulation step.

In some embodiments, the sample temperature (and optionally the temperature of all or part of the fluids in the assay) is thermostatically controlled, e.g., at 37° C. In some embodiments, sample is flushed out of the reaction loop to waste, and optionally wash fluid is circulated briefly.

In some embodiments, a pre-complexed mixture of a biotinylated secondary binding molecule (e.g., an antibody) and a labeled anti-biotin binding molecule (e.g., conjugated to gold particles) is circulated though a test chamber, assay chamber or channel of an assay chamber. The pre-complexed mixture may bind to reaction sites comprising a captured agent(s). In some embodiments, an alternative two-step method may be used and/or programmed, wherein attachment of a biotinylated secondary antibody, followed by a wash step, and then flowing anti-biotin-conjugated gold particles through a chamber or channel. In some embodiments, the above pre-complexed mixture may also comprise an agent of interest(s). In some embodiments, an agent may be pre-complexed with a detector binding molecule prior to binding a capture binding molecule. In some embodiments, the detector binding molecule may be directly or indirectly labeled.

In some embodiments, a final wash may be performed, e.g., leaving the chamber filled with wash fluid. In some embodiments, a sample may be read, along with a positive and a negative control(s).

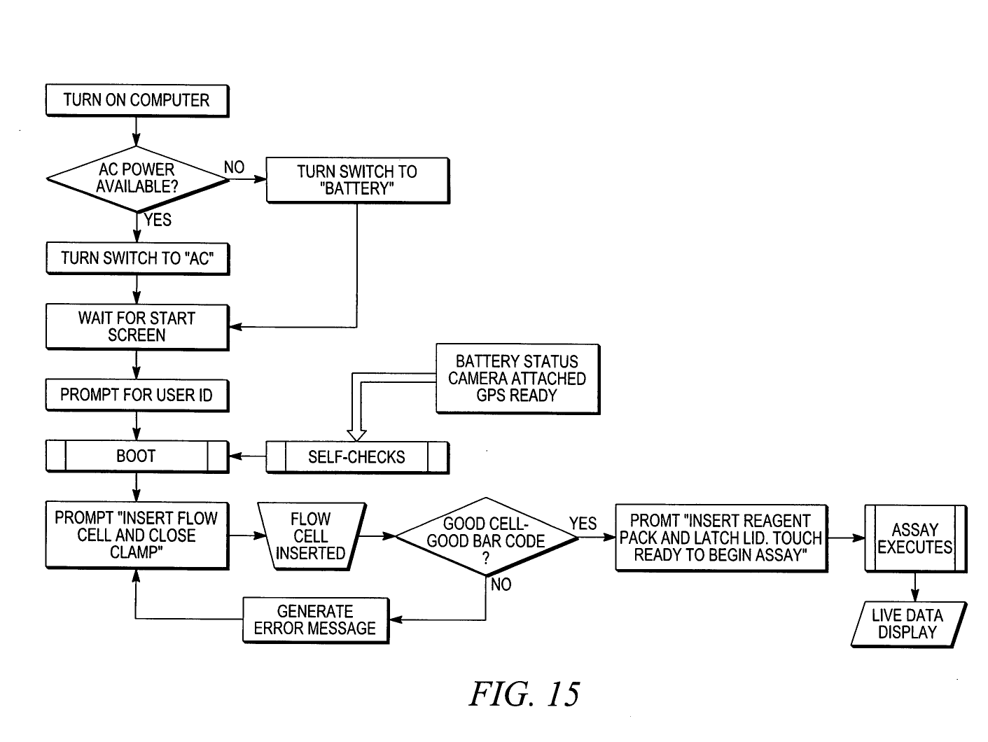

FIG. 15 shows an exemplary procedure (flow chart) for performing a detection assay(s) using some embodiments of a detection apparatus of the invention. In some embodiments, a device or detection apparatus is switched on (e.g. in AC or DC mode). In some embodiments, from this point forward, all user activity is prompted on the computer screen, e.g., via a GUI. In some embodiments, the detection apparatus or device performs self-checks, prompts a user for an ID, and/or leads a user through the assay steps. In some embodiments, a user interface, such as a GUI, is presented using a scripting language. A scripting language can allow for easy modification and updating, for example, as needed to accommodate new assays or protocols.

### Assay Formats

Assays of the invention may be designed to allow for the detection of one agent or more than one agent of interest (e.g., simultaneously) in a sample. The invention also provides assays for identifying, detecting and/or quantitating multiple compounds which interact with an agent of interest, such as, for example, to identify a peptide or other compound which binds an antibody, enzyme or cellular receptor of interest. Assays of the invention can also be used to screen for and identify compounds which catalyze chemical reactions, such as antibodies capable of catalyzing certain chemistries, and to screen for and/or identify compounds which give rise to detectable biological signals, such as compounds which bind to a receptor of interest. Interaction between an immobilized compound (e.g., acting as a capture binding molecule) and an agent gives rise to a detectable signal as described herein.

Some various assay formats and some related considerations are described in the Assay Guidance Manual Version 4.1, 2005, Eli Lilly and Company and NIH Chemical Genomics Center.

In some embodiments, the assay format may be a sandwich assay format. In some embodiments, a capture binding molecule(s) may bind an epitope on the agent and a labeled binding molecule(s) binds to an epitope on the agent. This is referred to as a direct sandwich assay format. This permits the agent to be “sandwiched” between the capture binding molecule and the label binding molecule. In some embodiments, the assay is an indirect sandwich assay format, e.g., wherein a labeled binding molecule is specific for a site, reporter group, or another binding molecule that is associated with the agent(s). For example, once an agent is captured, a biotinylated binding molecule may be used to “sandwich” the agent, and a biotin-specific labeled binding molecule is used. Thus, the invention includes compositions and methods which employ sandwich assays to identify, detect, or quantitate and agent or agents.

In some embodiments, an assay comprises (a) adding sample to a capture binding molecule(s) (e.g., an antibody or antibody array), (b) adding a secondary binding molecule(s) such as an antibody (e.g., conjugated to biotin) that binds a captured agent, and (c) adding a label (e.g., an RLS gold particle(s) conjugated to a binding molecule that binds to the secondary binding molecule(s) (e.g., an anti-biotin binding molecule such as avidin or an antibody that binds biotin). In some embodiments, an assay comprises (a) adding sample to a capture binding molecule(s) (e.g., an antibody or antibody array) and (b) adding a secondary binding molecule(s) such as an antibody that binds a captured agent wherein, the secondary binding molecule is directly labeled (e.g., with an RLS gold particle(s)).

In some embodiments of the invention, the assay is a competitive assay. In some embodiments, a labeled molecule is an analog(s) of an agent(s) of interest and the analog specifically binds with a binding molecule (e.g., a capture binding molecule) in competition with an agent(s). For example, a labeled molecule (e.g., with a LSL) is an agent-analog which competes with an agent(s), if any, in a sample for binding to a capture binding molecule. Thus, the detected signal (e.g., brightness of a spot) is inversely related to the quantity of an agent. In some embodiments, a sample and conjugate are typically mixed prior to contact with a reactive surface or a capture binding molecule. A LAM may be optionally used in competitive formats of the invention, just as in sandwich formats of the invention.

In some aspects of the invention, a labeled binding molecule may be specific for its respective partner (agent or other binding molecule, depending on the format) through intermediary cognate pairs. For example, if the agent is an oligonucleotide such as an amplification product bearing a binding reporter molecule (e.g., a hapten), a sandwich assay format might include a label conjugated to an antibody that binds the reporter molecule.

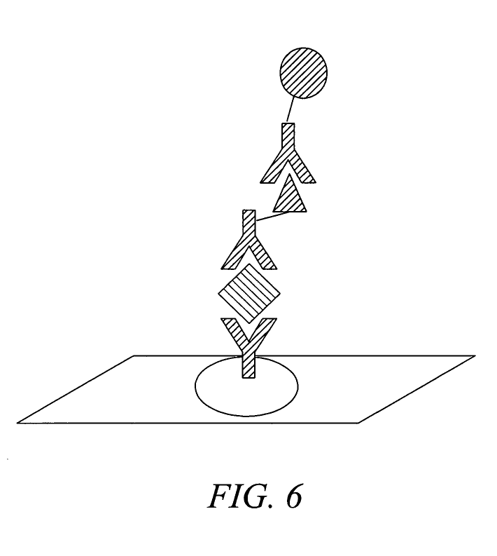

In some embodiments, an agent(s) is captured with a capture binding molecule(s). The agent may subsequently, previously or concurrently contacted with a second binding molecule(s) (a.k.a. a detecting binding molecule) comprising with an epitope or binding site for a third binding molecule (e.g., avidin/biotin) and wherein the second binding molecule(s) may be capable of binding (e.g., specifically) to the agent(s), e.g., see FIG. 6. The second binding molecule may subsequently, previously or concurrently contacted with a labeled binding molecule(s) which binds to the epitope or binding site.

In some embodiments, multiple detecting binding molecules may be utilized for a particular agent. For example, a capture binding molecule captures an agent and a detecting binding molecules (e.g., labeled) that bind different sites/antigens on the agent are utilized. This can give the advantage of binding multiple detectable binding molecules onto one captured agent, therefore increasing the number of labels associated with the agent, which in turn increases a detectable signal (e.g., light scattering or fluorescence) from the labels which can increase the sensitivity of an assay.

In some embodiments of the invention, a labeled binding molecule may be incubated with a sample of interest prior to contacting the sample with a reactive surface, e.g., containing a capture binding molecule.

Some embodiments of the invention utilize a binding molecule (e.g. an antibody) coupled with a nucleic acid(s) or a peptide nucleic acid(s) (PNA). In some of these embodiments, a nucleic acid is deposited or bound to a surface that can bind to a second nucleic acid or the PNA coupled to a binding molecule. In some embodiments, a binding molecule coupled with a nucleic acid(s) or a PNA binds an agent(s) of interest. In some embodiments, a binding molecule coupled with a nucleic acid(s) or a PNA binds another binding molecule that directly or indirectly binds an agent(s) of interest.

In some embodiments, a capture binding molecule can be composed of more than one molecule, wherein a first molecule is bound to or associated with to a surface and a second molecule binds to the first molecule and the combination of the first and second molecule bind to the agent. Some embodiments utilize the following format: (a) a first nucleic acid is bound to or associated with a surface; (b) a first binding molecule coupled with a second nucleic acid(s) or a PNA binds the first nucleic acid of (a); an agent is bound by the binding molecule of (b); and a second binding molecule is bound to the agent. In some embodiments, the second agent is directly or indirectly labeled. In some embodiments, the first and/or second binding molecule is an antibody such as a monoclonal antibody.

In some embodiments, an agent to be detected is a nucleic acid (e.g., RNA or DNA). In some embodiments of the invention, a nucleic acid is utilized as the capture binding molecule. This capture nucleic acid can be any nucleic acid such as DNA, RNA or a synthetic nucleic acid including synthetic DNA or RNA. In some embodiments of the invention a capture nucleic acid is between from about 0.01 to about 5 kb, about 0.01 to about 1 kb, about 0.01 to about 0.5 kb, about 0.01 to about 0.4 kb, about 0.01 to about 0.3 kb, about 0.01 to about 0.2 kb, about 0.01 to about 0.1 kb, about 0.01 to about 0.02 kb, about 0.015 to about 0.025 kb, about 0.02 to about 0.03 kb, about 0.025 to about 0.035 kb, about 0.03 to about 0.04 kb, about 0.035 to about 0.045 kb, about 0.04 to about 0.05 kb, about 0.045 to about 0.055 kb, about 0.05 to about 0.06 kb, about 0.055 to about 0.065 kb, about 0.06 to about 0.07 kb, about 0.065 to about 0.075 kb, about 0.07 to about 0.08 kb, about 0.075 to about 0.085 kb, about 0.08 to about 0.09 kb, about 0.085 to about 0.095 kb, about 0.09 to about 0.1 kb, about 0.095 to about 0.105 kb, about 0.1 to about 5 kb, about 0.5 to about 5 kb, about 1 to about 5 kb, about 2 to about 5 kb, about 3 to about 5 kb, about 4 to about 5 kb, from about 0.6 to about 1.2 kb, from about 0.5 to about 1.0 kb, from about 1.0 to about 1.5 kb, from about 1.5 to about 2.0 kb, from about 2.0 to about 2.5 kb, from about 2.5 to about 3.0 kb, from about 3.0 to about 3.5 kb, from about 3.5 to about 4.0 kb, from about 4.0 to about 4.5 kb, from about 4.5 to about 5.0 kb, from about 5.0 to about 5.5 kb, from about 0.1 to about 0.2 kb, from about 0.2 to about 0.3 kb, from about 0.3 to about 0.4 kb, from about 0.4 to about 0.5 kb, from about 0.5 to about 0.6 kb, from about 0.6 to about 0.7 kb, from about 0.7 to about 0.8 kb, from about 0.8 to about 0.9 kb, from about 0.9 to about 1.0 kb, from about 1.0 to about 1.1 kb, from about 1.1 to about 1.2 kb, from about 1.2 to about 1.3 kb, from about 1.3 to about 1.4 kb, from about 1.4 to about 1.5 kb, from about 1.5 to about 1.6 kb, from about 1.6 to about 1.7 kb, from about 1.7 to about 1.8 kb, from about 1.8 to about 1.9 kb, from about 1.9 to about 2.0 kb, from about 0.15 to about 0.25 kb, from about 0.25 to about 0.35 kb, from about 0.35 to about 0.45 kb, from about 0.45 to about 0.55 kb, from about 0.55 to about 0.65 kb, from about 0.65 to about 0.75 kb, from about 0.75 to about 0.85 kb, from about 0.85 to about 0.95 kb, from about 0.95 to about 1.05 kb, from about 1.05 to about 1.15 kb, from about 1.15 to about 1.25 kb, from about 1.25 to about 1.35 kb, from about 1.35 to about 1.45 kb, from about 1.45 to about 1.55 kb, from about 1.55 to about 1.65 kb, from about 1.65 to about 1.75 kb, from about 1.75 to about 1.85 kb, from about 1.85 to about 1.95 kb, or from about 1.95 to about 2.05 kb.

In some embodiments, an assay utilizes an array or mini array, e.g., a cDNA array, an RNA array, etc.

In some embodiments, one or more types of labels (e.g., metal or metal-like particles) are detected in a sample by measuring their emitted color or wavelength (e.g., under white light or similar broad band illumination) with illumination and detection methods, e.g., as described herein. In some aspects of the invention, roughly spherical particles of gold are coated with a binding molecule(s). In some embodiments, different particles (e.g., metal or metal-like), are detected and/or quantified in a sample by identifying each particle type by measuring the unique color/wavelength and/or the intensity of their respective scattered light. This can be carried out, for example on a solid phase or in solution. In some embodiments, the labels can be directly associated with the detecting binding molecules or indirectly associated, e.g., via another binding molecule that can bind the detecting binding molecule(s). Some assay methods of the invention employ total internal reflection (TIR) elements or waveguides as described herein and include, but are not limited to, competitive, direct or indirect sandwich assay formats.

In some embodiments of the invention, an agent, if any, in a sample undergoes an amplification step prior to and/or during a detection assay, e.g., wherein a nucleic acid(s) of interest, if present, is amplified. Typically, an amplification of an agent will increase the sensitivity of an assay. In some embodiments, during the amplification step the amplified product is designed to incorporate a tag(s) or detectable label(s). The tag can be essentially any molecule that can be bound by a binding molecule (e.g., biotin). In some embodiments, a tag could also be considered a detectable label. For example, fluorescein can be detected by fluorescence (label) and can also be detected using a binding molecule, e.g., using a labeled antibody that binds fluorescein. For example, a tag, could be, but is not limited to, biotin; fluorescein or other dyes; streptavidin or derivatives thereof; avidin or derivatives thereof; gold, silver, or other metal particles; plastic-like particles; electrically or magnetically charged materials or particles; oligonucleotides or other nucleic acids; antigens and antibodies; and enzymes and similar materials. In some embodiments, a nucleic acid is used to capture a nucleic acid agent with a tag and the captured nucleic acid agent is detected using a binding molecule that binds the tag. In some embodiments, the binding molecule that binds the tag is labeled (e.g., with a LSL, a fluorescent label or a quantum dot). In some embodiments, the binding molecule (labeled or unlabeled) that binds the tag is bound by a labeled “secondary” binding molecule. Amplification procedures include, but are not limited to, ligase chain reaction (LCR) or polymerase chain reaction (PCR)) and the labeled binding molecule is chosen to be specific for the reporter molecule. By “binding reporter molecule” is meant a molecule that can be bound by labeled binding molecule either via direct or indirect binding. In some of these and related embodiments, a capture binding molecule is a nucleic acid that binds an agent of interest. In some embodiments, the agent is a nucleic acid that hybridizes to the capture nucleic acid. In some embodiments, the nucleic acid agent is obtained via amplification from a sample. In some embodiments, the amplification process incorporates a “tag” that can be used to detect the nucleic acid agent. For example, the tag can be a fluorescent molecule. In some embodiments, the tag is a member of a binding partner pair and the other member is labeled.

In some embodiments, amplification products are optionally mixed with blockers, for example tRNA, Cotl DNA, or purified repeat sequences such as LINE or Alu sequences, or mixtures thereof. Nonnucleotide blocking agents can also be used, including proteins, for example BSA, caesin (e.g., 1% w/v Casein Hammersten Grade) and detergents. These blocking agents are not limited to nucleic acid detection assays.

In some assays of the invention, the detectable signal may be measured/detected at one or multiple points in time. One advantage of the present invention is that the detection or readout step of an assay can be performed at various times. This provides several benefits. For example, a detection assay typically provides incubation steps (e.g., in a sandwich assay) that have been optimized to provide a desired level of detection or sensitivity. Typically, longer incubation steps, up to a point, provide better or more sensitive levels of detection. In other words, to detect lesser amounts or concentrations of an agent may require longer incubation steps and higher amounts or concentrations of an agent may require shorter incubation steps for a signal to be detected. In many instances, these incubation steps may then incorporated into the final assay parameters and the detection step is performed at the end of the incubation step. The present invention provides the advantage of reading or detecting a signal(s) from a sample throughout an incubation step. Therefore, if a sample is strongly positive, the signal can be detected earlier. This provides various benefits including quicker processing of multiple samples and earlier detection of a harmful agent (e.g., a pathogen, toxin or pollutant). For example, in the field of biodefense, quicker detection and/or identification of a harmful agent can allow precautions to be taken earlier resulting in reducing the harmful effects or reducing the exposure of individuals to the harmful agent. Real time binding and/or dissociation can be monitored, e.g., visually or by video imaging, such as with a CCD camera, e.g., using software such as a frame grabber software.

In some embodiments, detectable signals may be measured/detected continuously, e.g., using a CCD camera and a computer. In some embodiments, detectable signals are measured at multiple time points. These time points can be any desired time points and may vary depending on the assay and agent to be detected.

In some embodiments of the invention, a capture binding molecule (e.g., an antibody) that binds (e.g., specifically) to an agent(s) of interest is immobilized or attached to a surface. Then a sample is contacted with the capture binding molecule so that an agent of interest present in the sample can bind the capture binding molecule. A second binding molecule (detector binding molecule) is used to also bind the agent. (e.g. see FIG. 6) The second binding molecule can be labeled directly or indirectly. In these and some other embodiments, the specificity of the capture binding molecule and the second binding molecule vary. For example, at least one of the capture or detector binding molecules typically can be specific for an agent of interest. In some embodiments, the combination of the capture and detector binding molecules can be specific and/or the combination leads to the specificity. For example, the capture binding molecule may bind to numerous (usually related) agents, e.g., different strains of a bacteria or different serotypes of a virus. The detector binding molecule may also bind to numerous (usually related) agents, but only one agent or a group of agents of interest will be bound by both the capture and detector binding molecules. Some embodiments of the invention provide methods and compositions for distinguishing typically cross-reactive agents.

In some embodiments, the capture binding molecule can bind a class or family of agents or multiple agents. In this embodiment, a population of detector binding molecules can be used wherein members of the population bind different agents and these members can be separately detected, e.g., each member is labeled (directly or indirectly) with a different light scattering particle which results in distinct detectable signals. This can allow for the detection of multiple agents in one assay. In some embodiments, a bottom (capture) binding molecule can bind multiple agents while the top (detector) binding molecules can bind different agents, e.g., at least two different populations of binding molecules each associated with different labels (e.g., different particles or different fluorescent labels or a combination thereof). In some embodiments, different capture binding molecules are located in distinct regions or sites on a surface. In some embodiments, different capture binding molecules are located in close proximity, e.g., spotted in the same solution on a glass slide.

Some embodiments of the invention include mixing of a fluid sample after bringing it in contact with a reactive surface. Although, mixing may not be required, mixing may help to ensure close contact between the fluid sample and an immobilized binding molecule. In some embodiments, a flow (e.g., capillary flow) of sample fluid across a reactive surface is utilized. This can provide the benefits of enhanced contact and binding of an agent(s) to a capture binding molecule. In some embodiments, a sample and/or assay reagents are circulated in a “loop” for a period of time over the reactive surface or capture binding molecules.

In some embodiments of the invention, a sample of interest may be “processed” or undergoes a sample preparation method/process prior to performing an assay, e.g., as described herein.

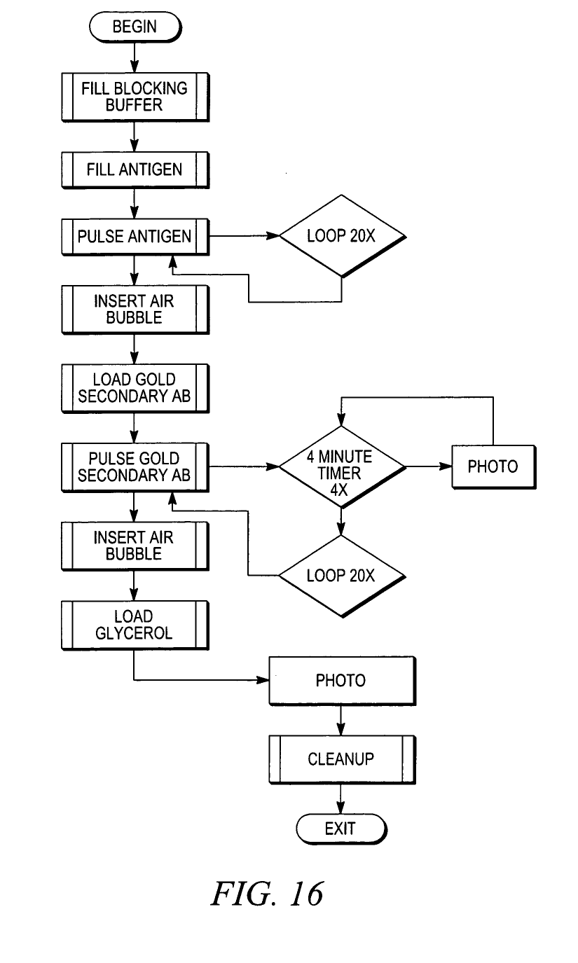

An exemplary procedure is represented by the flow chart in FIG. 16. In some embodiments, a apparatus performs a “prewetting” step, which also places the positive controls into the flow path. In some embodiments, a user is prompted to insert a reagent pack and a assay chamber (not shown in FIG. 16). In some embodiments, a user is prompted to inject an unknown antigen. In some embodiments after this point, all machine operation is automatic. In some embodiments, steps of the assay are programmed in a scripting language, for example, that is easily modified to accommodate new assays or new protocols, as required. FIG. 16 shows as an example monitoring every 4 minutes during development, but this can be done at any desired interval, or continuously.

### Assays for Multiple Agents

When designing an assay(s) for detecting multiple agents, an early step can be to decide which agents or categories of agents the test will identify, distinguish, quantitate and/or detect. For example, to construct an assay to identify agents or a category of agents that cause pneumonia, one may select agents (e.g., pathogens) that commonly cause pneumonia, such as Respiratory Syncytial Virus and Streptococcus pneumoniae; or, to test for food borne pathogens, one might select agents (e.g., bacteria) or category of agents that cause a food borne illness(es). Methods and compositions of the invention can be used to identify a broad range of agents. Furthermore, various types of agents (e.g., bacteria, toxins and/or viruses) can be tested for in a single assay, typically simultaneously. In some embodiments, agents (e.g., infectious agents) or a category of agents selected for an assay are known to be present in a particular geographical location.

The components of assay chambers and detection apparatuses for analyzing multiple agents are described in detail elsewhere herein. In brief, some assay formats of the invention can include the capability to analyze multiple agents and in some cases simultaneously. In some embodiments, an assay chamber or reactive surface comprises different populations binding molecules that bind different agents, e.g., in the form of an array.

In some embodiments, the different populations of binding molecules are “spotted” together and the detection and/or distinction of individual agents may be accomplished by populations of detector binding molecules labeled (directly or indirectly) with labels. These labels can, for example, produce detectable and distinguishable signals. For example, these labels may be fluorophores with different emission wavelengths or LSLs capable of scattering/emitting different emission wavelengths.

In some embodiments, different agents may be detected on different areas, regions or sites of an assay chamber or reactive surface, e.g., as a typical array. For example, different populations of capture binding molecules are spotted in different sites, respectively. In these embodiments, different populations of labeled detector binding molecules for each agent may have the same or different labels, since the different agents may be detected in different/distinguishable sites.

Some embodiments, of the invention utilize “category-binding molecules” and/or assays that detect categories of agents. The term “category-binding molecules” is a set of binding molecules that bind to one or more members of a category of agents. For example, polyclonal antibodies raised to a Hepatitis C virus can be a family of antibodies or “category-binding molecules” since it comprises multiple binding molecules that bind to the same category of agents, in this case HCV. Another example of category-binding molecules is a set of category-specific genomic DNA, for example, sequences that occur in all E. coli O157:H7 strains, but do not occur in members of other groups of bacteria. These category-binding molecules can hybridize as a group to nucleic acids from E. coli O157:H7 cells or and typically does not significantly hybridize to other types of cells. Category-binding molecules and/or assays of the invention can be directed against a specific disease and/or symptoms. Thus, the invention includes methods and compositions for detecting, identifying or quantitating agents in a category.

In some embodiments, a set of category binding molecules is utilized, e.g., as either the capture binding molecules or the detecting binding molecules or both. In some assays of the invention, a capture binding molecule or capture binding molecules are used that bind a category of agents, e.g., Dengue I, II, III and IV viral antigens. In some of these embodiments, the detector binding molecules all comprise the same label and all bind the category of agents. In some embodiments, the detector binding molecules comprise labels with are labeled differently based on the agent(s) they bind, therefore, allowing discrimination of different agents within a category. In some embodiments, a category can be of binding molecules or assays that bind agents in a particular sample type, e.g., plant pathogen (e.g., affecting a particular plant type and/or found in a geographic region), bioweapon agents, chemical weapon agents, food pathogens or toxins, blood metabolites, etc. This results in an assay that detects the presence of an agent in the category. In another embodiment, a capture binding molecule(s) binds a category of agents and at least two different detector binding molecules are utilized which bind at least two different agents, wherein the at least two different detector binding molecules are labeled or capable of being labeled (e.g., indirectly) with different labels that are distinguishable. For example, a capture binding molecule (e.g., a monoclonal antibody) is used that binds various or even all serotypes of a particular virus (e.g., adenovirus). The agent(s) is then bound to the capture binding molecule. In some embodiments, at least two “detecting” binding molecules that are each specific for different serotypes are bound to the captured agents and the at least two detecting binding molecules are each labeled with different labels that allow for the distinguishable detection (e.g., simultaneous) of each of their respective agents (e.g., serotypes). In the case of LSLs, different labels can differ based on, for example, size or shape or composition. In the case, of fluorescent labels (e.g., quantum dots) the labels can differ by their absorbance λ, emission λ, or both.

In some embodiments, an assay is a pneumonia test. For example, an ensemble of binding molecules (e.g., antibodies) that react to category-specific antigens related to pneumonia is used. For example, these category-specific antigens could be on the surface of microbes that cause pneumonia or internal (e.g., nucleic acids). A binding molecule or set of binding molecules in this category-binding molecule ensemble might comprise polyclonal antibodies from the immunoglobulin fraction of antiserum raised in a host (e.g., rabbit, mouse or goat) and directed against Streptococcus pneumoniae. In another embodiment, another set of binding molecules in this category could comprise a recombinant antibody or a monoclonal antibody directed against a coat protein of adenovirus. Streptococcus pneumoniae and adenovirus are both known agents that can cause or contribute to pneumonia.

In some embodiments, categories of one or more agents will be chosen for an assay, assay chamber or array. A category of agents can comprise 2 or more agents. Categories of agents include, but are not limited to, food borne pathogens, pathogens found in a particular geographic location, categories of possible bioweapon agents (e.g., those known or suspected to be possessed by another party), pathogens that cause pneumonia or pneumonia-like symptom, pathogens that cause a particular symptom or set of symptoms, different antibodies related to vaccinations (e.g., a group of vaccinations and individual has received), etc.

In some embodiments, a category of agents are those listed by the CDC Emergency Preparedness & Response as Bioterrorism Agents/Diseases Category A, B and/or C. These categories are believed to be periodically updated, so the invention includes categories encompassing past or current Bioterrorism Agents/Diseases Category A, B and/or C.

In some embodiments, agents to be detected by an assay or detection apparatus of the invention comprise those capable of causing one or more of the following: 1) Anthrax (e.g., Bacillus anthracis); 2) Botulism (e.g., Clostridium botulinum toxin); 3) Plague (e.g., Yersinia pestis); 4) Smallpox (e.g., variola major); 5) Tularemia (e.g., Francisella tularensis); or 6) Viral hemorrhagic fevers (e.g., filoviruses (e.g., Ebola, Marburg) and arenaviruses (e.g., Lassa, Machupo)).

In some embodiments, the agents detected by an assay or detection apparatus of the invention comprises one or more of the following: (1) Bacillus anthracis protective antigen (PA); (2) B. anthracis lethal factor (LF); (3) B. globigii (BG); (4) ricin; (5) Clostridium botulinum toxins A/B; and (6) Staphylococcal enterotoxin B (SEB). In some embodiments, the agents detected by an assay or detection apparatus of the invention consists of (1) B. anthracis protective antigen (PA); (2) B. anthracis lethal factor (LF); (3) B. globigii (BG); (4) ricin; (5) C. botulinum toxins A/B; or (6) Staphylococcal enterotoxin B (SEB).

In some embodiments of the invention, an assay or detection apparatus of the invention detects 1, 2, 3, 4, 5, 6, 7, 8, 9, 10, 11, 12, 13, 14, 15, 16, 17, 18, 19, 20 or more related or unrelated agents. In some embodiments, an assay or detection apparatus of the invention detects between from about 1 to about 20, about 2 to about 20, about 3 to about 20, about 4 to about 20, about 5 to about 20, about 5 to about 15, about 10 to about 20, about 1 to about 10, about 1 to about 15, about 1 to about 5, about 2 to about 10, about 3 to about 10, about 4 to about 10, about 5 to about 10, about 1 to about 5, about 10 to about 15, about 15 to about 20, about 1 to about 20, about 15 to about 30, about 30 to about 50, about 1 to about 10,000, from about 1 to about 1,000, from about 1 to about 100, from about 20 to about 100, from about 50 to about 100, from about 75 to about 100, from about 20 to about 30, from about 20 to about 40, from about 30 to about 50, from about 40 to about 60, from about 50 to about 70, from about 60 to about 80, from about 70 to about 90, from about 80 to about 100, from about 100 to about 200, from about 200 to about 300, from about 300 to about 400, from about 400 to about 500, from about 500 to about 600, from about 600 to about 700, from about 700 to about 800, from about 800 to about 900, from about 900 to about 1000, from about 1,000 to about 2,000, from about 2,000 to about 3,000, from about 3,000 to about 4,000, from about 4,000 to about 5,000, from about 5,000 to about 6,000, from about 6,000 to about 7,000, from about 7,000 to about 8,000, from about 8,000 to about 9,000, or from about 9,000 to about 10,000 different agents.

In some embodiments, an assay of the invention is capable of detecting an agent (e.g., a protein) present in a sample wherein the concentration of the agent is between from about 1 picogram(pg)/ml to about 1 μg/ml, about 1 pg/ml to about 100 ng/ml, about 1 pg/ml to about 10 ng/ml, about 10 pg/ml to about 10 ng/ml, about 100 pg/ml to about 100 ng/ml, about 100 pg/ml to about 10 ng/ml, about 100 pg/ml to about 1 ng/ml, about 1 ng/ml to about 100 ng/ml, about 1 ng/ml to about 10 ng/ml, about 1 ng/ml to about 5 ng/ml, about 5 ng/ml to about 10 ng/ml, about 10 ng/ml to about 20 ng/ml, about 20 ng/ml to about 30 ng/ml, about 30 ng/ml to about 40 ng/ml, about 40 ng/ml to about 50 ng/ml, about 50 ng/ml to about 60 ng/ml, about 60 ng/ml to about 70 ng/ml, about 70 ng/ml to about 80 ng/ml, about 80 ng/ml to about 90 ng/ml, about 90 ng/ml to about 100 ng/ml, about 1 pg/ml to about 1 ng/ml, about 1 pg/ml to about 100 pg/ml, about 1 pg/ml to about 10 pg/ml, or about 10 pg/ml to about 100 pg/ml.

Therefore, the present invention provides methods for detecting multiple agents or categories of agents. The present invention also provides compositions (e.g., assay chambers, reactive surfaces and/or detection apparatuses) for detecting multiple agents or categories of agents.

### Assay Controls

The present invention provides method and compositions related to controls and/or providing controls for an assay.

If an assay yields a negative result, it is often important, but not always, to know whether the sample is truly free of an agent or whether the assay itself failed or did not function properly, e.g., whether or not the result is a false negative. To identify false negative results, one or more positive control agents can be utilized. In some embodiments, a positive control agent or portion thereof is added to an experimental sample. In some embodiments, a positive control agent is captured by the same capture binding molecules as the test agent. In some embodiments, a positive control agent contains binding sites that do not occur in the range of agents being tested and/or the positive control is “captured” using a capture binding molecule that is different from capture binding molecules used for analyzing a sample(s).

In some embodiments, control samples are incorporated into a sample chamber of the assay. In some embodiments, a control sample or control material is contacted with an assay chamber or reactive surface of the invention. In some embodiments where an assay is conducted in a flow chamber, controls are deposited “upstream” of capture antibodies. In some embodiments, the controls are deposited or dried in such a way that when a solvent/liquid (e.g., an assay buffer/reagent or a sample for analysis) is introduced (e.g., into a channel containing a control) into this chamber or channel, the control composition is transported to the capture binding molecules. The control can be negative, positive, or of known concentrations, e.g., for correlating an amount of an agent in a sample with a known quantity. These embodiments provide a simple and accurate means for including a control(s) for an assay and at the same time minimizes the amount of steps and reagents necessary to perform the assay.

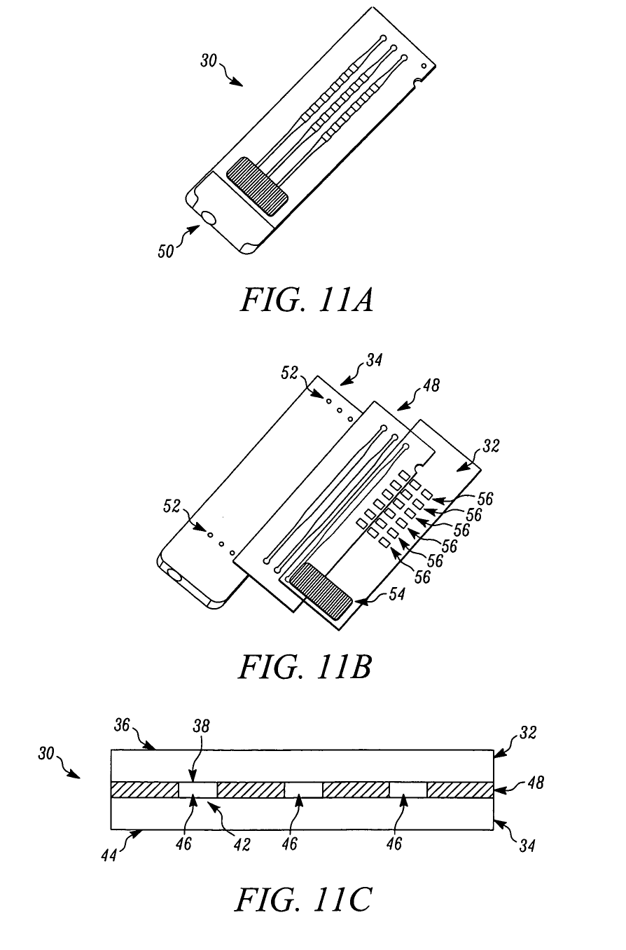

In some embodiments, a control material is deposited (e.g., by micropipette) onto an assay chamber in a flow channel upstream of a situs. In some embodiments, this deposition is performed after application of a gasket, but before a array slide or waveguide element is attached. The control material is allowed to air dry. In some embodiments, it is stored under desiccating vacuum conditions until an assay chamber is assembled. In some embodiments, control material is deposited a waveguide element and/or on a superstructure element, e.g., see FIG. 11. A control material may be a positive or negative control material. Concentrations and amounts of a deposited control material will vary depending upon the agent(s) and or sample types. The following are exemplary amounts of control material the may be deposited for a corresponding agent: recombinant Bacillus anthracis protective antigen 2 μg; Bacillus globigii spores 3 μg; Staphylococcal enterotoxin B (SEB) 45 ng; Clostridium botulinum Type A Complex toxoid 5 μg; Ricin A chain 1 μg; and Inactivated Yersinia pestis 10 μg.

In some embodiments, a flow cell with 3 chambers is utilized as the assay chamber, e.g. see FIG. 11. In some embodiments, one channel is for a sample. In some embodiments, a second channel is for a positive control. In some embodiments, one channel is for a negative control. In some embodiments, all three (positive control, negative control and at least one sample) are inputted into their respective channel by a user, e.g., via a syringe. In some embodiments, the negative and/or positive sample is already contained within their channel of the flow cell. In some embodiments, a user will manually introduce a solvent/liquid that transports a control composition to the capture binding molecule. In some embodiments, a detection apparatus automatically and/or via a pump mechanism, introduces a solvent/liquid that transports a control composition to the capture binding molecule. In some embodiments, a control composition is already attached and/or deposited in an assay chamber. In some embodiments, on the sample is inputted into an assay chamber.

In some embodiments, an assay comprises a control which includes detecting an control agent that is present in all of the samples. In some embodiments, a control agent is an agent different from the agent being detected in a sample. For example, it is a control for the general assay methods, but not necessarily for the particular assay. Binding molecules corresponding to the positive control targets are included, e.g., with the other binding molecules used in the assay. These targets will be detected in all assays, unless one or more of the assay steps is unsuccessful. Failure to detect a signal from a positive control thus can indicate or suggest a false negative result. In some embodiments, the assay comprises a binding molecules (e.g., in a situs) that bind to an agent (a control agent) naturally present in a sample e.g., IgG antibodies if the sample is serum or a ubiquitous plant protein if the sample is plant tissue. In some embodiments, the binding molecules, e.g., of a positive situs, bind an agent (e.g., biotin or avidin) that is “spiked” into a sample, therefore acting as a positive control for the assay.

Therefore, the present invention provides methods related to quality control and confirming assay results. Additionally, the present invention provides methods for delivering a control sample or control agent to an assay, assay chamber, reactive surface or detection apparatus.

### Samples and Agents

Essentially any one or more agents from essentially any sample(s) can be detected using the present invention. An agent can be detected, quantitated, analyzed, and/or identified from a sample or the sample can be processed, for example as described herein, prior to analysis. In some embodiments, an agent is selected from the group consisting of a gram positive organism, a gram negative organism, a gram indefinite organism, a prion and a prion-like agent, an emerging infectious agent, a biologically or chemically mutated or altered agent, a yeast, a parasite, a bacteria and a virus (e.g., capable of infecting man, plants, insects and animals), contaminating agents (e.g., yeast, parasites, bacteria and viruses) in environmental samples such as water, air, and food. Nucleic acids (e.g., a DNA or RNA), proteins, peptides, antigenic fragments or epitopes of any of the aforementioned organisms or cells can be detected utilizing the methods and compositions of the present invention. The present invention also contemplates that any agent described herein can be considered as a binding molecule and therefore, could be utilized, e.g., as a binding molecule, a capture binding molecule, a direct detector binding molecule or a secondary binding molecule.

An agent can be an organism, virus or complex organism or a detectable portion thereof, e.g., a nucleic acid of a pathogen. A non-limiting list of agents includes, but is not limited to, a protein or peptide, a toxin such as Botulinum, Epsilon toxin of Clostridium perfringens or ricin toxin, a B. anthracis protective antigen (PA), a B. anthracis lethal factor (LF), a B. globigii (BG), a C. botulinum toxin A or B, a Staphylococcal enterotoxin B (SEB), a viral protein, a virus capable of animal infection and/or disease, a BVDV (Bovine virus diarrhea), a IBR (Bovine Rhinotrachetis), a PI-3 (Parainfluenza), a BPV (Bovine Parvovirus), a BAV (Bovine Adenoviruses), a BpoV (Bovine Polyomavirus), a BMV (Bovine Mammilitis virus), a FMD virus (Foot & Mouth Disease Virus), a VSV (Vesicular Stomatitis Virus), a Orf Virus, a BEV (Bovine Enterovirus), a PEV (Porcine Enterovirus), a PPV (Porcine Parvovirus), a Rabies Virus, a REO-3, a BRSV (Bovine Respiratory Syncytial Virus), a PHV-1 (Porcine Herpes virus-1), a Rhinovirus, a Calicivirus, a Rotavirus, a Hog Cholera, a Border Dis., an EEE (Eastern Equine Encephalitis Virus), a WEE (Western Equine Encephalitis Virus), a VEE (Venezuelan Equine Encephalitis Virus), a JEE (Japanese Equine Encephalitis Virus), a Akabane virus, a BTV (Blue tongue virus), a virus capable of human infection and/or disease (e.g., a Herpes Simplex Virus-1,2, a HAV (Hepatitis A), a HBV (Hepatitis B), a HCV (Hepatitis C), a HEV (Hepatitis E), a HIV-1,2 (AIDS) , a parvovirus B-19, a Adenovirus, a Poxvirus (e.g., Smallpox or a vaccinia), a RSV (Respiratory Syncytial), a Measles virus, a Rubella virus, an Influenza virus (e.g., a A or B or H5N1 strain), a Parainfluenza virus, a Mumps virus, a Rabies virus, a HTLV, a CMV (cytomegalovirus), a Poliomielitos virus, a Arbovirus, a Hantavirus, a Nipah virus, a MFV (Marburg fever virus), an Ebola virus, a Lassa virus, a Calicivirus, a Coxsackie virus, a rotavirus, a reovirus (e.g., type 1, 2, or 3), a papovavirus (e.g., Simian Virus-40), a Polyomavirus, a Papillomavirus, a Rhinovirus, a Yellow Fever virus, a Dengue virus, a Encephalitis virus, a Corona virus, a Varicella-Zoster virus, an Epstein-Barr virus, an Adenovirus, an African Swine Fever Virus, an Arbovirus, an Alphavirus, an Arenavirus, an Arterivirus, an Astrovirus, a Bacteriophage, a Baculovirus, a Bunyavirus, a alicivirus, a Caulimovirus, a Coronavirus, a Filovirus, a Flavivirus, a Hepadnavirus, a Herpesvirus, a Myovirus, a Nodavirus, an Orthomyxovirus, a Paramyxovirus, a Papovavirus, a Parvovirus, a Phycodnavirus, a Picornavirus , a Poxvirus, a Reovirus, a Retrovirus, a Rhabdovirus, a Togavirus, a prokaryotic protein, a mammalian protein (e.g., a cellular receptor, a cytokine, an IL-1, an IL-2, an IL-3, an IL-4, an IL-5, an IL-6, an IL-7, an IL-8, an IL-9, an IL-10, an IL-11, an IL-12, an IL-13, an IL-14, an IL-15, an IL-16, an IL-17, GM-CSF, IFN (e.g., alpha or gamma), TNF (e.g., alpha), an allergen, a nucleic acid (e.g., from any source such as a cell (e.g., an animal, mammalian, primate, non-human primate or human cell)), an infectious agent (e.g., a virus or a bacteria (e.g., of the genus Staphylococcus, Streptococcus, Corynebacterium, Bacillus, Neisseria, Shigella, Escherichia, Salmonella, Klebsiella, Proteus, Erwinia, Vibrio (e.g., cholerae), Pseudomonas, Brucella, Bordetella, Haemophilus, Yersinia, Burkholderia mallei, or Burkholderia pseudomallei)), Chlamydia psittaci, Coxiella burnetii, Rickettsia prowazekii, Listeria, Legionella species, verocytotoxin producing E. coli (VTEC) serotypes (e.g., O157, O145, O111, O103 and O26), M. pneumoniae, Corynebacterium diphtheriae, Eschericia coli, Streptococcus pyogenes, Staphylococcus aureus, Mycobacteria tuberculosis, a mycoplasma (e.g., M. bovimastitidis, M canis, M. hominis, M. hyorhinis, M. urealyticum, M. orale, M. salivarium, M. laidlawi), a yeast cell, Saccharomyces cerevisiae, Cryptococcus neoformans, Blastomyces dermatitidis, Histoplasma capsulatum, Paracoccidiodes brasiliensis, and Candida albicaus, a fungus, Coccidioides immitis, Aspergillus fumigatis, Microsporum audouini, Trichophyton mentagrophytes, and Epidermophyton floccosum, glucose, a vascular endothelial growth factor (VEGF), a PDGF, an environmental agent (e.g., a pollutant), a chemical, a plant pathogen (e.g., a viral, a fungal, a bacterial or a fungal-like plant pathogen), a potato pathogen, a Verticillium (e.g., dahliae), a Phytopthora (e.g., infestans or erythroseptica), a Clavibacter (e.g., michiganensis; e.g., subspecies sepedonicus), an Erwinia (e.g., carotovora), a Streptomyses (e.g., scabiei), a Fusarium (e.g., oxysporum), a Helminthosporium (e.g., solani), a P. infestans, a Ralstonia (e.g., solanacearum), a Pectobacterium (e.g., atrosepticum), a Pythium (e.g., ultimum), a Xyllela (e.g., factidiosa), a Cryptosporidium (e.g., parvum), a Giardia (e.g., intestinalis or lamblia), a Salmonella, a Potato Virus Y, a Potato Virus X, a pathogen or toxin affecting an aquaculture product (e.g., a fish, a salmon, a kelp, a sea weed, a shellfish (such as an oyster, clam, mussel, etc.), a shrimp, a crustacean (such as a crab, a blue crab, a lobster, etc.)), a cell surface receptor, an intra-cellular receptor, an intra-cellular signaling protein, a G-protein coupled receptor, an ion channel, an enzyme (e.g., a protease, ubiquitinase, deubiquitinase, or kinase), a DNA binding protein, a metabolite (e.g., glucose and urea), a sexually-transmitted pathogen, an agent causing a blood infection or sepsis, an inorganic molecule, a macromolecule, a parasite, a hormone, a cell type (e.g., a cancer cell), a food pathogen, an illegal drug, a legal drug, a drug of abuse, an antibody (e.g., IgG, IgE, IgM, IgD, IgA, IgY, IgG1, IgG2, IgG3, IgG4, IgA1 or IgA2), a pharmaceutical agent, a vaccine, an antigen, an immunogen, an allergen, an emerging infectious agent of man or animal, or a prion-like agent.

In some embodiments, a sample to be tested is or from a bodily fluid sample, blood, urine, cerebrospinal fluid, sputum, tissue samples or feces. Examples of a sample include, but are not limited to, blood, urine, semen, milk, sputum, mucus, a buccal swab, a vaginal swab, a rectal swab, an aspirate, a needle biopsy, a section of tissue obtained, for example, by surgery or autopsy, plasma, serum, spinal fluid, lymph fluid, the external secretions of the skin, respiratory, intestinal, and genitourinary tracts, tears, saliva, tumors, organs, samples of in vitro cell culture constituents (including, but not limited to, conditioned medium resulting from the growth of cells in cell culture medium, putatively virally infected cells, recombinant cells, and cell components), plant cells or tissues, water samples, air samples, soil samples or a recombinant source, e.g., a library comprising polynucleotide sequences, polypeptides or peptides. In some embodiments, a sample is a water sample, an air sample, a dirt sample, a swab, and/or a swipe.

Insects can be vectors for many types of diseases and infectious agents. In some embodiments, a sample is from an insect or collection/pool of insects. An insect(s) (e.g., roaches, mosquitoes, ticks, flies, spiders, fleas, sand fleas, etc.) can be processed by various methods known in the art, e.g., crushing, extraction, grinding, and/or pulverizing. In some embodiments, a sample is analyzed to detect an animal pathogen (e.g., an arbovirus or an alphavirus). Insects can be collected from one or various locations. Extracts from insects can be pooled or multiple insects and/or insect types can be extracted or processed together, e.g., as a pool. Thus, the invention provides methods for detecting an agent present in or associated with an insect(s). The insect associated agent may not necessarily be a pathogen, but could be, for example, an insect protein or nucleic acid.

In some embodiments, a detection apparatus, assay chamber and/or method of the present invention can be utilized to analyze or evaluate an animal's immune response (e.g., antibody levels) for a particular agent(s), e.g., an infectious agent(s) or vaccine component(s). These embodiments can be utilized, for example, for the following non-limiting examples: to detect and/or determine levels of antibodies against a particular antigen (e.g., a pathogen or toxic chemical in blood or serum); to determine the effectiveness of a vaccine (e.g., administered with or without an adjuvant); to determine if antibody levels are sufficient for protection; to detect auto-antibodies (e.g., related to or known to be markers for a disease); to detect IgE antibodies that bind an antigen(s) (e.g., for allergen testing); to determine levels of neutralizing antibodies; to determine if a vaccine and/or booster vaccine is warranted; to determine if an animal has been exposed to a particular antigen(s) by the presence of antibodies, to determine levels of a certain antibody type (e.g., IgG, IgE, IgM, IgD, IgA and IgY), a class (e.g., IgG1, IgG2, IgG3, IgG4, IgA1 and IgA2) or subclass of immunoglobulin molecule; or to determine levels of a certain type, a class, or subclass of immunoglobulin molecule that is directed against an antigen(s). Antibodies can be from any source including, but not limited to, blood, serum, swabs, nasal secretion, lung secretion, sputum, lymph node, thymus or hybridoma. Antibodies can be from any animal species including, but not limited to, a bird, a mammal, a mouse, a human, a goat, a bovine, a donkey, a guinea pig, a camel, a chicken, a sheep, a dog, a cat, a horse, a rat, a hamster or a rabbit.

In some embodiments, a panel of antibody levels can be detected. In some embodiments, a panel of antibody levels related to a panel of vaccines can be detected. For example, if a person has been given a number of vaccines, one can test their serum for antibodies directed to each of the pathogens the vaccinations were directed against. In some embodiments, analysis can be performed using an assay chamber that detects a panel of antibodies directed against a panel of pathogens and determines which antibody levels are low enough to recommend a vaccine or booster vaccine. In some embodiments, an assay chamber is coated with multiple (e.g., an array) binding molecules that each comprises at least one epitope from a pathogenic organism. For example, an assay chamber comprises multiple sites comprising a binding molecule, wherein each situs is comprised of epitopes to bind antibodies for a particular pathogen or category of pathogens. Using FIG. 11 as an example, each channel comprises 6 sites. In some embodiments, each of these six sites is an antigen(s) for a pathogen or vaccine candidate, so as the assay detects antibodies to six different pathogen or vaccine candidate. In some embodiments, a capture binding molecule is a protein(s), peptide(s), or combination thereof, wherein the protein(s), peptide(s), or combinations thereof comprise an antigenic site(s) or epitope(s) for a pathogen(s) or vaccine candidate(s). In some embodiments, multiple antigenic sites or epitopes from a pathogen are utilized to bind antibodies that bind different epitopes of the pathogen. In some embodiments, a certain epitope or certain epitopes of a pathogen are known to be neutralizing. By “neutralizing” is meant that when an antibody is directed to this epitope, it typically eliminates or significantly inhibits the detrimental effects of the pathogen, e.g., prevents or inhibits attachment and/or internalization. In some embodiments, an assay of the present invention comprises the use of neutralizing epitopes to bind or capture antibodies directed to neutralizing epitopes. In some embodiments, only known neutralizing epitopes are utilized. In some embodiments, an assay of the invention detects antibody levels to certain pathogens in a geographic region, e.g., an assay chamber comprises multiple binding sites (e.g., an array) comprising antigens found in a specific geographical region. Some parts of the foregoing discuss, as examples, the detection of antibodies, typically from serum, that bind a pathogen, e.g., a pathogen for which an animal has been vaccinated. For clarity the assays can be utilized to detect antibody responses or a panel/array of antibody responses directed against any vaccine candidate or any immunogenic compound, e.g., a protein toxin, a chemical toxin, or a chemical) and is not limited to antibodies against pathogens.

In some embodiments, antibody levels are measured by a detection apparatus, an assay chamber or a method of the invention for the purposes of determining if an animal(s) (e.g., a human, livestock or wild animal) has been exposed to a pathogen or toxin. In some embodiments, a capture binding molecule can bind antibodies from an animal that bind a pathogen(s) or toxin(s) of interest to see if the animal has been exposed (e.g., recently) to a particular pathogen, toxin or antigen. The detected antibodies can be of any type (e.g., IgG, IgE, IgM, IgD, IgA and IgY), class (e.g., IgG1, IgG2, IgG3, IgG4, IgA1 and IgA2) or subclass of immunoglobulin molecule. The type and level of antibody response can be use to determine or estimate, e.g., the time of exposure and/or the severity of exposure. Additionally, some embodiments of the invention can be used track the course of exposure to an agent (e.g., a pathogen) and the resulting antibody response over time. For example, high levels of specific IgM antibodies are typically present on for a few weeks after an initial exposure and as IgM levels drop off, typically IgG levels will rise for the corresponding antigen. Some embodiments of the invention provide an assay or assay chamber for detecting an agent and antibodies to an agent. In some embodiments, an assay will detect any antibody type (e.g., IgG, IgM or IgA) to an agent. In some embodiments, an assay will detect each or at least two antibody types against an agent(s) and optionally detect the agent itself

In some embodiments, an array of allergens is utilized as capture binding molecules. In some embodiments, a sample (e.g., serum or blood) is contacted with the array of allergens and then bound IgE is detected, e.g., using an anti-IgE binding molecule (e.g., directly or indirectly labeled). These embodiments are useful for allergy testing/screening.

In some embodiments, an assay for detecting antibodies that bind an antigen(s) comprise a) a capture binding molecule (e.g. a peptide or protein) containing an antigen(s) or epitope(s) thereof; b) a sample possibly containing or suspected to contain an antibody(s) of interest that binds the capture binding molecule (directly or indirectly); and c) a second binding molecule that binds the antibody(s) of interest. In some embodiments, the second binding molecule is directly and/or indirectly labeled. In some embodiments, the second binding molecule can bind a type(s), class(es), or subclass of antibody. In some embodiments, capture sites with different antigens are used, wherein the second binding molecule (detector antibody) is the same for detecting antibodies bound to any or all of the sites. For example, to detect IgM antibodies to different antigens, sites each comprising different antigens are used to capture IgM antibodies from a sample (e.g., serum). Then a second binding molecule that binds IgM antibodies is utilized, wherein the second binding molecule is directly or indirectly labeled. Therefore, the invention provides methods for detecting and/or quantitating binding of antibodies to an antigen. The invention also provides methods of measuring and/or analyzing binding interactions between an antibody and an antigen. Some embodiments of the invention provide methods for measuring neutralizing antibodies directed against a pathogen. Also, provided are methods for analyzing, assessing or measuring antibodies to a particular antigen and/or vaccination from an animal. Also, provided are methods for detecting an animal's exposure to a pathogen, antigen or toxin comprising measuring and/or detecting antibodies (e.g., IgG, IgA, and/or IgM) from the animal that bind an antigen related to the pathogen or toxin. The invention also provides methods for measuring and/or detecting auto-antibodies in an individual.

In some embodiments, an antibody against an antigen and the antigen itself can be detected or analyzed in the same assay or assay chamber of the invention. For example, an assay chamber comprises at least two capture sites, wherein one capture situs comprises the antigen or an epitope thereof and a second capture situs comprises a binding molecule that binds the antigen. The antigen and an antibody that binds the antigen are detected from a sample as described herein. In some embodiments, an assay chamber comprises at least one channel comprising two capture sites. In some embodiments, an assay chamber comprises at least two channels wherein each contains one situs of the two capture sites. Therefore, the present invention provides methods for detection an antigen itself and antibodies (e.g., simultaneously) that bind the antigen.

Some embodiments of the invention provide methods, apparatuses and compositions for measuring, identifying or detecting an agent that is a marker (e.g., a surrogate marker) indicative of an event or biological activity. A marker can be, but is not limited to, a protein or nucleic acid of a biological agent of interest; a biological protein or nucleic acid whose levels increase, decrease or remain the same in response to an event such as a viral infection. Markers include those for detecting the presence of a cell type or the presence of cancerous cells (e.g., prostate specific antigen (PSA) or alpha-fetoprotein (AFP)). Markers also include those for pharmacogenomics. Therefore, the present invention provides methods for diagnosing different conditions in an animal. Also provided are methods for detecting a marker of an event, e.g., a biological marker.

Some embodiments of the invention provide methods, apparatuses and compositions for measuring, identifying or detecting an agent related to the general status of an individual or sample. For example, some embodiments of the invention provide methods, apparatuses and compositions for measuring, identifying or detecting an agent related to transplantation. Agents can include, cellular proteins (e.g., on the cell membrane), antigenic epitopes, antibodies directed against a particular epitope(s), a major histocompatibility complex (MHC) (e.g., for MHC typing), or infectious agents. Therefore, the present invention provides methods for screening a transplantation organ for an infectious agent and/or for compatibility with a recipient. Some embodiments of the invention are well suited for this purpose due, in part, to the possible portability of some detection apparatuses; the possible rapid assay analysis; the possible exchangeable assay chamber and/or assay reagents; and the ability to transmit data (e.g., wirelessly). Embodiments related to transplantation include an allograft, autograft or xenograft. Also, provided are methods for MHC and or HLA typing an individual.

Some embodiments of the invention are useful, in addition to human medicine, in veterinary medicine and/or analysis of animal samples, e.g., from an animal patient. An individual includes humans as well as other animals such as veterinary animals. Advantages of some embodiments of the present invention with regards to use by a veterinarian or an animal researcher are the portability of a detection apparatus and the ability to change an assay chamber cassette depending on the agent to be analyzed. Some embodiments of the invention are particularly suited for use by an epidemiologist of the like, e.g., in the field. Again the portability of a detection apparatus is beneficial, but also the ability to analyze various sample types with one apparatus. In some embodiments, a user can analyze various animal samples (e.g., humans, non-human mammals, and insects), food samples, and water samples, for example, when investigating an outbreak using a single detection apparatus of the invention.

Some embodiments of the detection apparatuses and/methods of the invention also find use in the areas of remediation and/or decontamination. For example, to determine if an agent has been sufficiently reduced in or removed from an area. Areas can be tested after and optionally before or optionally during remediation to determine if an agent(s) of interest is below, above or at acceptable levels of contamination. In some embodiments, an area is tested prior to remediation to determine what remediation steps will be taken.

An apparatus(s), assay(s) and/or method(s) of the present invention finds use in various settings and fields. In some embodiments, an apparatus(s), assay(s) and/or method(s) of the present invention is utilized at point-of-care or on-site. For example, an apparatus(s), assay(s) and/or method(s) of the invention is utilized to test patient samples for an agent(s). In some embodiments, an apparatus(s), assay(s) and/or method(s) of the present invention allows a user to perform diagnostic tests outside of dedicated laboratories. Although, they can be utilized in a dedicated laboratory. In some of these embodiments, an apparatus(s), assay(s) and/or method(s) of the invention is utilized in close proximity to a patient. In some embodiments, an apparatus(s), assay(s) and/or method(s) of the invention is utilized in a, physician office, hospital, veterinarian's office, a laboratory, nursing home, public or private health clinic, college health center, correctional facility, emergency vehicle, a workplace, a home, in the same room or building that a sample was obtained or bedside. Additionally, the type of sample can dictate places or locations that a sample is analyzed. For example if a water sample is to be analyzed, analysis can take place in the field (e.g., next to a body of water, at a water reservoir, water treatment facility, water intake or discharge area) or in another location (e.g., central lab). In some embodiments, an apparatus(s), assay(s) and/or method(s) of the invention is utilized for analyzing environmental samples. In some embodiments, an apparatus(s), assay(s) and/or method(s) of the invention is utilized to detect an infectious agent(s), a potential bioweapon(s), an environmental toxin(s), a pollutant(s), a contaminant(s) (e.g., in a water sample or source), a food pathogen or a contaminant, a plant pathogen or combinations thereof. In some embodiments, these analyses are performed at a location within close proximity to where a sample was retrieved. In some embodiments, samples are collected and/or embodiments of the invention are performed on means of transportation, e.g., in or on a car, bus, truck, train, trailer, airplane, space craft, boat, military ship, submarine, etc. Some embodiments of the present invention provide a portable (e.g., typically capable of transportation or carrying by one person) apparatus/device for analyzing agents. In some embodiments, analyses or detection of the invention is performed in military field operations, on training grounds, or on a battlefield (friendly, non-friendly). In some embodiments, analyses or detection is performed by a government agency, e.g., a laboratory, a federal funded research and development center, or a contractor's site.

The detection apparatuses of the invention are particularly suited for combination with other automated methods and devices. In some embodiments, a detection apparatus, sample chamber or method of the invention can be utilized in combination with a drone, robot, plane (e.g., manned or unmanned), boat/ship (e.g., manned or unmanned), submarine (e.g., manned or unmanned) or spaceship (e.g., manned or unmanned). As an example, a detection apparatus can be completely automated, which then can be used in one of the foregoing. Some embodiments of the invention have the advantage of being operational at zero G's or in low or altered gravity environments, e.g., for operation in space. Therefore, some embodiments of the invention are utilized for the detection of an agent(s) in space or outside of the earth's atmosphere. Therefore, the present invention provides methods for remotely collecting and analyzing a sample.

Embodiments of the invention may be utilized by first responders, e.g., for biowarfare agent detection. Some embodiments are utilized for routine and possibly automated detection of building air and/or water supply.

In some embodiments, a detection apparatus of the invention is utilized to test the possible contamination of a crop or a food source, for example, with a pathogen (e.g., E. coli) or toxic agent. Crops can be tested at any point before and even after delivery to a consumer. Some crops are washed with a solution prior to delivery to a consumer. For example, apparatuses and methods of the invention may be used to test a crop in the field (e.g., before harvest); after harvest; or before, during and/or after washing. For example, a detection apparatus or method of the invention can be used to analyze washing solution prior to, during and/or after washing. In some embodiments, a washing solution is continually used and/or repeatedly used for multiple washings of the same or different plants. This washing solution can be tested/sampled at various time points. Methods of the invention can be used to test crops and/or wash solutions throughout the wash process to detect a contamination, such as with E. coli early in the process, possibly preventing shipment to consumers and further contamination of other crops, for example, that are processed at the same location. In some embodiments, samples (e.g., pant, animal, human samples, etc) are tested for an agent such as an E. coli strain, e.g., O157, O145, O111, O103 and/or O26.

Some embodiments of the invention allow for the analysis of binding interactions or biomolecular interactions and in some cases kinetic or real time analysis can be conducted. Some embodiments of the invention, as described herein, provide a detection apparatus that can record images in real time or at various time points. Is some scientific applications, e.g., related to antibody/antigen or receptor/ligand interactions, it is desirable to analyze binding characteristics of two molecules and in some instances under different conditions. For example, an application may require analyzing the binding characteristics of a binding molecule such as an antibody under different conditions such as varying pH. This can be investigated utilizing an apparatus of the invention. For example, a corresponding antigen is utilized as the capture binding molecule. Then an antibody that binds the antigen is contacted with the antigen. This assay can be repeated several times under different conditions (e.g., pH) or can be run once and the conditions changed, e.g., while monitoring the binding characteristics. In some embodiments, the same concentration of antibody is maintained throughout the assay. In some embodiments, an antibody can be contacted with a capture antigen under conditions that allow the antibody to bind. Then a solution(s) with different characteristics (e.g., changes in pH, ionic strength, and/or presence of a binding competitor) can be introduced into the assay chamber and the amount of bound antibody can be determined and/or monitored over time and under the different conditions. Therefore, the present invention provides methods of measuring and monitoring binding characteristics of a binding molecule or pair of binding molecules. In some embodiments, more than one condition is changed, e.g., pH and ionic strength. Some embodiments take advantage of a circulation loop to change the conditions. For example, a chemical(s) can be introduced into the circulating loop and the binding characteristics are monitored, e.g., continuously or at time points. This can allow for a gradual change in a condition or a gradient analysis. For example, HCL can slowly be added to the recirculating solution to allow for a gradual decrease in pH while monitoring the binding characteristics.

The present invention can also be utilized to analyze drug interactions and or for drug screening. In some embodiments, a capture binding molecule is a cellular receptor (e.g., a G-protein coupled receptor) and binding of a ligand (e.g., a natural ligand, a small molecule, a drug, a drug candidate, an antibody, etc.) in analyzed or detected. In some embodiments, an array of capture binding molecules such as cellular receptors and/or a potential drug targets are utilized in an assay chamber or on a reactive surface. In some embodiments, a compound or compounds are contacted with the array and analyzed, typically to detect which capture binding molecules are bound and sometimes to what extent. In some embodiments, an assay is a competitive assay. For example, an array of capture binding molecules such as cellular receptors and/or a potential drug targets are utilized in an assay chamber or on a reactive surface. Then a compound or compounds are contacted with the array along with a known ligand. Detection of bound ligand(s) is utilized to determine if a drug competes with binding of the ligand for the capture binding molecule. Other embodiments can determine protein to protein interactions such as using a protein and/or peptide array and contacting with a protein(s) and/or peptide(s) of interest. Similarly, nucleic acid arrays can be analyzed such as cDNA arrays or the like can be utilized to analyze mRNA from a cell.

Some embodiments of the invention can be utilized to, for example: optimize binding conditions for an assay of the invention or even another assay type; measure binding properties and a binding dissociation constant(s); screen binding molecules for a particular dissociation constant; search for binding partners; screen for inhibitor specificity (e.g., competitive assay); remove contaminants or inhibiting substances; test for cross-reactivity; look for activity after concentration, measure quantity of a biological or biological response, determine quality of a biological or biological response; detect activity after partial purification, detect activity after purification; or test cells (e.g., cells from in vitro, ex vivo, or in vivo) for the expression of a given protein, nucleic acid or other biological.

### Sample Preparation

One characteristic of the invention is its compatibility with various methods of sample preparation, although many embodiments and assays of the invention do not require any sample preparation. A sample can be diluted, dissolved, suspended, extracted or otherwise treated, e.g., to solubilize and/or purify any agent present or to render it accessible to reagents which are used in an assay. Where a sample contains cells, the cells may be lysed or permeabilized to release an agent within a cell. One step permeabilization buffers can be used to lyse cells which allow further steps to be performed, e.g., directly after lysis, for example, an amplification step, a concentration step, a purification step and/or detection analysis.

Sample preparation can have several functions depending on the nature of the sample and the assay format. For example, a sample may be processed to concentrate, dilute, filter, purify and/or amplify an agent prior to analysis/detection. In some embodiments, an amplification step is followed by a concentration step. In some embodiments, a sample is filtered prior to analysis, e.g., to remove particles of a size that can interfere with detection and/or clog the detection apparatus. An exemplary filtration apparatus for removal of particles from a sample is a manually operated PURADISC Syringe filter by Whatman. (Whatman Inc., Florham Park, N.J.)

In some embodiments, samples are pooled and then tested. This can reduce the number of samples to be analyzed. Pooling of sample prior to testing can be used for essentially any type of sampling. In some embodiments, when a pooled sample tests positive for an agent the sample are individually tested and/or tested in subpools. For example, an area (e.g., a building or city) is suspected to be contaminated with an agent(s) or has been contaminated with an agent and was processed for decontamination. Samples (e.g., swabs, air samples, water samples, and/or samples from animals) can be pooled into one or more pools, e.g., 100 samples divided in to 10 pools each containing 10 samples. If one or more of the 10 pools is positive for an agent(s) of interest, then one can go back to the original samples from a positive pool and test them individually to determine what area are contaminated. For example if only one of the pooled samples is positive, the positive sample can be determined by only processing 20 samples, not 100. Of course pooling and the degree of pooling will depend on the nature of the samples, desired detection levels, and assay formats and sensitivity of the assay.

Some embodiments of the invention comprise amplifying a sample or agent. In some embodiments where an agent is an infectious agent, such as a virus or bacteria, the infectious agent may be amplified in or from the sample prior to analysis in an assay. For example, a sample suspected to contain a virus may be contacted with cells in which the virus can replicate. After an appropriate incubation time, a sample may be harvested from the cells and assayed as described herein. In some embodiments where a bacterium is to be detected, a sample can be used to culture under appropriate conditions a bacterium, if present. After an appropriate incubation time, a sample may be harvested from the culture and assayed as described herein. Other infectious agents may be amplified in a similar manner and then assayed. Another means of amplifying an agent in a sample would be to amplify a nucleic acid associated with an agent and then assay for the amplified nucleic acid. Typically, amplification of an agent in a sample would be used when the amount and/or concentration of an agent is below and/or thought to be below the detectable limit of the corresponding assay(s). Amplification methods also include, but are not limited to, a polymerase chain reaction method (PCR), a ligase chain reaction (LCR), self sustained sequence replication (3SR), nucleic acid sequence-based amplification (NASBA), the use of Q Beta replicase, reverse transcription, nick translation, and the like. An amplification step can optionally be followed by a concentration step. A concentration step can optionally be followed by an amplification step. Therefore, the invention provides methods for amplifying an agent or sample. Also provided are methods for amplifying an agent prior to analysis or detection of the agent. Also provided are methods for increasing assay sensitivity comprising amplifying an agent in a sample, e.g., for an assay of the invention or other assay. Amplification can occur before and/or during an assay of the invention, e.g., a detection assay.

In some embodiments, an assay or method of the invention comprises amplifying an agent while detecting or monitoring the agent using an apparatus or assay chamber of the present invention. For example, an apparatus of the invention can PCR amplify a nucleic acid and production of product can be monitored during the amplification. In some embodiments, amplification occurs in an assay chamber. In some of these embodiments, detection of the amplified agent is detected and or monitored during or through out the assay, e.g., at various points or continually. In some embodiments, amplification occurs outside of an assay chamber. In some of these embodiments, aliquots from the reaction are tested during the amplification. Aliquots can be manually or automatically introduced into the assay.

Concentration of an agent(s) suspected to be present in a sample can be performed a number of ways including, but not limited to, evaporation, filtration, centrifugation, affinity binding (e.g., column affinity chromatography, beads (e.g., magnetic such as paramagnetic or superparamagnetic beads, e.g., from Invitrogen, Carlsbad, Calif.) attached to binding molecules capable of binding the agent(s)), immuno-magnetic separation, or centrifugation methods. In some cases, a sample preparation concentrates an agent and/or deposits it on a substrate. In some embodiments, sample preparation methods for an agent(s) in a liquid (e.g., water-borne microbes) will concentrate the agent by filtration, depositing an agent on a filter. In some embodiments, a sample is concentrated via filter centrifugation, e.g., using a Centricon concentrator device or the like such as a Centricon YM30 (Millipore, Billerica, Mass.). Centrifugation methods for concentration also include pelleting an agent away from a portion of a sample or removing a portion of a sample containing the agent from a pellet. Additionally, concentration can occur by using gradient centrifugation where the agent localizes to a portion of the gradient which can be separated from the rest of the sample/gradient.

In some embodiments, concentration of an agent of interest comprises the use of particles (e.g., beads). In some embodiments, particles (e.g., beads) comprise a binding molecules that binds an agent. In some embodiments, agents are concentrated from a sample using agent specific binding molecules attached to particles. In some embodiments, a binding molecule (e.g., an antibody) that binds an agent comprises a first member of a binding partner (e.g., biotin) and a particle or bead is comprised of a second member of a binding partner (e.g., streptavidin). In some embodiments, a particle comprises a first binding molecule (e.g., streptavidin) and an antibody comprises a second binding molecule (e.g., biotin), wherein the antibody binds an agent of interest and wherein the second binding molecule binds the first binding molecule. In some embodiments, a particle or bead comprises a “secondary” binding molecule which binds a “primary” binding molecule that binds an agent. In some embodiments, a particle or bead is comprised of streptavidin (e.g., Catalog#'s 110-47, 602-10, and 653-05, Invitrogen, Carlsbad, Calif.). In some embodiments, a bead or particle binds a nucleic acid. In some embodiments, monoclonal and/or polyclonal antibodies are attached to particles or beads.

In some embodiments, concentration of an agent of interest comprises the use of beads or particles. In some embodiments, concentration of an agent of interest comprises automated or a manual addition of beads or particles (e.g., magnetic). In some embodiments, beads or particles are coated with a binding molecule(s) (e.g., an antibody) that binds an agent in a sample. In some embodiments, beads or particles comprising binding molecules are contacted with a sample. In some embodiments, the contacting is performed with agitation. In some embodiments, the contacting is followed by separation of the agent bound particles or beads. In some embodiments, a binding particle or bead is incubated or contacted with a sample for an incubation time of about 1, 2, 3, 4, 5, 6, 7, 8, 9, 10, 11, 12, 13, 14, 15, 16, 17, 19, or 20 minutes. In some embodiments, a binding particle or bead is incubated with a sample for an incubation time between from about 1 minute to about 24 hours, from about 1 minute to about 16 hours, from about 1 minute to about 12 hours, from about 1 minute to about 8 hours, from about 1 minute to about 6 hours, from about 1 minute to about 4 hours, from about 1 minute to about 2 hours, from about 1 minute to about 1 hour, from about 1 minute to about 50 minutes, from about 1 minute to about 40 minutes, from about 1 minute to about 30 minutes, from about 1 minute to about 20 minutes, from about 1 minute to about 15 minutes, from about 1 minute to about 10 minutes, from about 1 minute to about 5 minutes, from about 5 minute to about 10 minutes, from about 10 minute to about 15 minutes, from about 15 minute to about 20 minutes, from about 20 minute to about 25 minutes, from about 25 minute to about 30 minutes, from about 30 minute to about 35 minutes, from about 35 minute to about 40 minutes, from about 40 minute to about 45 minutes, from about 45 minute to about 50 minutes, from about 55 minute to about 60 minutes, from about 1 hour to about 1.5 hours, from about 1 hour to about 2 hours, from about 1 hour to about 4 hours, from about 2 hours to about 5 hours, from about 4 hours to about 8 hours, from about 8 hours to about 12 hours, from about 12 hours to about 16 hours, from about 16 hours to about 20 hours, from about 20 hours to about 24 hours, from about 16 hours to about 36 hours, from about 24 hours to 48 hours, or from about 48 hours to about 72 hours.

In some embodiments, a particle is a polymeric particle. The particles or beads can be composed of the same polymer throughout, or they can be core-shell polymers as described, for example, in U.S. Pat. No. 4,847,199, 4,703,018, and 5,284,752; and European Patent Publication No. EP0280556, e.g., where the shell polymer has the requisite reactive groups.

To aid manipulation and separation of immobilized material, and also to facilitate automation if required, some embodiments of the invention utilize magnetisable (“magnetic”) particles or beads. The term “magnetic” as used herein means that a support or bead is capable of having a magnetic moment imparted to it when placed in a magnetic field, and thus is displaceable under the action of that field. In other words, a particle or bead comprising magnetic material may readily be removed from other components of a sample by magnetic aggregation, which typically provides a quick, simple and efficient way of separating particles or beads. In addition, such magnetic aggregation is typically a less rigorous method of separation than traditional techniques, such as centrifugation, which can generate shear forces which may disrupt cells or degrade some other moieties, e.g., proteins or nucleic acids bound to a particular particle or bead.

In some embodiments, a particle is a polymeric particle containing ferromagnetic crystals, superparamagnetic crystals or a mixture thereof. Magnetic polymer particles are known and may, for example, be prepared and/or utilized according to the processes described in, e.g., U.S. Pat. No. 4,654,267; 5,232,782 5,763,203; and 5,814,687. In some embodiments, the present invention can utilize particles (e.g., beads) comprising paramagnetic, non superparamagnetic and/or superparamagnetic crystals. Paramagnetic particles will typically exhibit slight magnetic remanent properties. Non-superparamagnetic crystals are remanent in the sense that, upon exposure to a magnetic field, the material will have residual magnetization in the absence of a magnetic field. Superparamagnetic polymeric particles are magnetically displaceable but are not permanently magnetizable which can avoid magnetic remanence and possible clumping. In some embodiments, superparamagnetic crystals may be of any material capable of being deposited in superparamagnetic crystalline form in and/or on the polymeric particles. In some embodiments, magnetic particles (e.g., magnetic beads) are monodisperse (i.e., are substantially uniform in size, e.g., size having a diameter standard deviation of less than 5%) to typically provide uniform kinetics and separation. In some embodiments, a particle (e.g., bead) is spherical and/or monodisperse. Preparation of superparamagnetic monodisperse particles is described, for example, in U.S. Pat. No. 4,774,265.

Embodiments utilizing magnetic particles or beads typically involve contacting magnetic particles or beads capable of binding (e.g., directly or indirectly) an agent in a sample, allowing the beads to bind an agent(s), if present in the sample, and exposing the beads/sample to a magnet or magnetic field to separate the magnetic particles or beads from a portion of the sample.

In some embodiments, a bead or particle is capable of being immobilized on an immobilizing moiety, e.g., a solid support. This immobilization to a solid phase allows easy manipulation of the bead or particle and the bound agent, if any. Attachment to a solid phase can enable the separation of the components from the rest of the components in the mixture. This can be achieved for example by carrying out washing steps, or if the agent(s) is attached to magnetic beads or magnetic particles, using a magnetic field to effect physical separation of the linked component from the rest of the components in the mixture. Thus, magnetic particles (e.g., beads) with a bound agent(s) may be isolated onto a suitable surface by application of a magnetic field, e.g., using a magnet. It is usually sufficient to apply a magnet to the side of a vessel containing a sample mixture to aggregate particles (e.g., magnetic beads) to the wall of the vessel and to remove the remainder of the sample so that the remaining sample and/or the particles are available for any further steps.

A solid support may be any of the well-known supports or matrices which are used for immobilization, separation etc., in chemical or biochemical procedures. These may take the form of particles, sheets, dip-sticks, gels, filters, membranes, microfibre strips, tubes, wells or plates, fibres or capillaries, combs, pipette tips, microarrays or chips or combinations thereof, and conveniently may be made of a polymeric material, e.g., agarose, sepharose, cellulose, nitrocellulose, alginate, Teflon, latex, acrylamide, nylon membranes, plastic, polystyrene, glass or silica or metals. Numerous suitable solid supports are commercially available.

The well-known monodisperse polymeric magnetic beads sold by Invitrogen Dynal AS (Oslo, Norway) under the trade mark Dynabeads™, are exemplary of commercially available magnetic particles which may be used or modified for use according to the invention.

In some embodiments, a bead or particle is non-magnetic. Non-magnetic beads or particles suitable for use in the present invention are, for example, available from Invitrogen Dynal AS (Oslo, Norway) under the trademark Dynospheres, as well as from Qiagen, GE Healthcare Life Sciences, Serotec, Seradyne, Merck, Nippon Paint, Chemagen, Promega, Prolabo, Polysciences, Agowa and Bangs Laboratories.

An agent binding molecule may be covalently attached to a particle or bead through reactive groups on the substrate surface by methods known in the art. These include, for example, attachment through hydroxyl, carboxyl, aldehyde or amino groups which may be provided by treating the particle to provide suitable surface coating.

Supports with functionalized surfaces are commercially available from many manufacturers, such as those particle manufacturers described herein. Magnetic particles with the following functionalized surfaces are available, e.g., from Invitrogen (Dynal AS, Oslo, Norway), and are utilized in some embodiments of the present invention: Hydrophobic beads; Dynabeads® M-450 Epoxy (with epoxy groups); Dynabeads® M-450 Tosylactivated (with tosyl groups); Dynabeads® M-280 Tosylactivated (with tosyl groups); Dynabeads® MyOne Tosylactivated (with tosyl groups); Dynabeads® M-500 Subcellular (with tosyl groups); Hydrophilic beads; Dynabeads® M-270 Epoxy (with epoxy groups); Dynabeads® M-270 Carboxylic acid (with carboxylic acid groups); Dynabeads® MyOne Carboxylic acid (with carboxylic acid groups); or Dynabeads M-270 Amine (with amino groups).

The appropriate choice of surface may depend on the type of moieties which are to be attached. An attachment can be achieved through amino or sulfhydryl groups on a binding molecule which are available for reaction directly with reactive groups on the outer surface of the particles. There are many useful reactive groups which react with a free amine group of a binding molecule. Such groups include, but are not limited to, carboxy, active halogen, activated 2-substituted ethylsulfonyl, activated 2-substituted ethylcarbonyl, active ester, vinylsulfonyl, vinylcarbonyl, aldehyde, epoxy, amino and sulfhydryl. Some of these groups will react directly with a binding molecule (e.g., an antibody) while others, such as carboxy, require the use of a compound to produce an intermediate which will react with a binding molecule. Reagents suitable for crosslinking of the solid surface of a particle (e.g., a bead) and a binding molecule include cyanogen bromide, carbonyldiimidazole, glutaraldehyde, hydroxysuccinimide and tosyl chloride. In some embodiments, a Tosyl- or epoxy surface is used.

In some embodiments, particles or beads are utilized that are tosylactivated and/or are coated with forms of epoxy, carboxylic acid, or amines. Invitrogen (Carlsbad, Calif.) provides kits with appropriately derivatized beads and chemicals, buffers, and procedures to attach binding molecules to particles or beads via different functional groups.

Particles or beads that bind essentially any agent(s) can be utilized for the concentration an agent(s). Some embodiments of the invention utilize particles or beads that bind Legionella (e.g., Dynabeads® anti-Legionella, Catalog#730-03, Invitrogen, Carlsbad, Calif.), E. coli O157 (e.g., Dynabeads® anti-E. coli O157, Catalog#710-03, Invitrogen), E. coli O103 (e.g., Dynabeads® EPEC/VTEC O103, Catalog#710-11, Invitrogen), E. coli O111 (e.g., Dynabeads® EPEC/VTEC O111, Catalog#710-09, Invitrogen), E. coli O145 (e.g., Dynabeads® EPEC/VTEC O145, Catalog#710-07, Invitrogen), E. coli O26 (e.g., Dynabeads® EPEC/VTEC O26, Catalog#710-13, Invitrogen), Listeria (e.g., Dynabeads® anti-Listeria, Catalog#710-06, Invitrogen), and/or Salmonella (e.g., Dynabeads® anti-Salmonella, Catalog#710-02, Invitrogen).

In some embodiments, a method may be performed using an automated system for handling of such magnetic particles. Some embodiments of the invention combine a detection method with automated concentration/purification, e.g., to aid with detection of agents from clinical, environmental, and other samples. The sample containing an agent may be transferred to such an apparatus, and magnetic particles carrying, e.g., binding molecules against an agent(s), can be added. In some embodiments, processing and/or concentrating with particles or beads utilizes partial or complete automation, e.g., using a BeadRetriever™ magnetic bead processor, Invitrogen, Carlsbad, Calif. In some embodiments, an apparatus has a system for ready and efficient transfer of a support carrying particles or beads from one well to another. Magnetic particles or beads are described herein as exemplary particles or beads that can be utilized in accordance with the present invention. However, the invention is not limited to the type of particles or beads since any particle or bead can be used that binds an agent(s) and subsequently allows for concentration and or purification of the agent bound beads. For example, a nonmagnetic or a magnetic particle or bead can typically be concentrated from an aqueous sample, for example using centrifugation.

In some embodiments, once an agent is concentrated using particles or beads the agent can be released from the particle or bead and analyzed according to the invention. For example, if an agent is bound to an antibody associated with a bead in a solution. In some embodiments, the solution can be changed or modified (e.g., ionic strength and/or pH change) to release the agent into the solution. This agent containing solution can then be run directly in an assay or the conditions of the solution can be modified (e.g., return to neutral pH) prior to the sample analysis. In some embodiments, an agent is analyzed without release from a particle or bead. In some embodiments, a particle is labeled allowing for detection of the agent using methods as described herein.

In some embodiments, a binding molecule will be attached to a bead or particle, wherein the bead or particle is between from about 0.1 μm to about 100 μm, about 0.1 μm to about 10 μm, about 0.1 μm to about 1 μm, about 1 μm to about 100 μm, about 1 μm to about 10 μm, about 1 μm to about 5 μm, about 5 μm to about 10 μm, about 1 μm to about 2 μm, about 2 μm to about 3 μm, about 3 μm to about 4 μm, about 4 μm to about 5 μm, about 5 μm to about 6 μm, about 6 μm to 7 μm, about 7 μm to about 8 μm, about 8 μm to about 9 μm, or about 9 μm to about 10 μm. In some embodiments, a population of particles or beads (e.g., in the size ranges described above) is utilized wherein the average size has a standard deviation of about ±0.05 μm, ±0.1 μm, ±0.2 μm, ±0.3 μm, ±0.4 μm, ±0.5 μm, ±0.6 μm, ±0.7 μm, ±0.8 μm, ±0.9 μm, or ±1.0 μm. In some embodiments, a particle or bead (e.g., magnetic) is about 0.5 μm, about 0.6 μm, about 0.7 μm, about 0.8 μm, about 0.9 μm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, 4.1 μm, about 4.2 μm, about 4.3 μm, about 4.4 μm, about 4.5 μm, about 4.6 μm, about 4.7 μm, about 4.8 μm, about 4.9 μm, or about 5.0 μm. In some embodiments, the diameter or average diameter of a bead or particle is of at least 0.01 μm and/or has a maximum diameter of not more than 10 μm or not more than 6 μm. In some embodiments, the diameter or average diameter of a bead or particle is about 1.0 μm, about 2.8 μm or about 4.5 μm.

Concentration, purification and/or detection of agents using beads or particles is described, for example in Bead Retriever User Manual Rev. 03, Mar. 5, Invitrogen, Carlsbad, Calif.; Demnerova et al., Microbiology 3(4):225-9 (2000); Docherty et al., Lett Appl Microbiol. 22(4):288-92 (1996); Guillot et al., Journal of Microbiology Methods 54(1):29-36 (2003); Hartig et al., Electrophoresis 16(5):789-92 (1995); Li et al., Journal of Food Protection, 66(3):512-7 (2003); Lai et al., Crit Care Med. 33(12 Suppl):S433-4 (2005); Lim et al., Clin Microbiol Rev. 18(4):583-607 (2005); Monteiro et al., J Clin Microbiol. 39(10):3778-80 (2001); Nundy et al., Journal of Food Protection 61(11):1507-10 (1998); Petrenko et al., J Microbiol Methods 58(2):147-68 (2004); Siddons et al. Epidemiol Infect 113(1):31-9 (1994); Taylor et al., Vet Microbiol. 56(1-2):135-45 (1997); Uhlen et al., Clinical Microbiology Review 7(1):43-54 (1994); Widjojoatmodjo et al., J Clin Microbiol. 30(12):3195-9 (1992); Wolfe et al., Applied and Environmental Microbiology February: 841-845 (1999); Wolfbagen et al., J Clin Microbiol. 32(7):1629-33 (1994); Xu et al., J Biomed Opt. 10(3):031112 (2005); or Yazdankbah et al., Vet Microbiol. 67(2):113-25 (1999).

Considerations and suggestions for methods of concentrating and/or isolating agents using agent binding particles or beads include, but are not limited to, typically use filtered pipette tips for sample transfer and additional pipette manipulations; typically vortex beads before use to provide a homogenous mixture (the beads are typically only vortexed at this point, and not after introduction to the sample); typically if dealing with an extremely viscous or fatty sample a dilution may be required to dilute the sample, e.g., with the specified wash buffer (e.g., PBS-Tween); typically use a 360° rotational mixer (e.g., 25-30 rpm with top to bottom rotation) for the incubation periods because flat bead mixers or rotational plate shakers can allow the matrix to settle around the beads, instead of actively mixing, which can increase nonspecific binding; typically optimize incubation times keeping in mind that increasing incubation will typically only slightly increase agent recoveries, but can greatly increase the potential for nonspecific binding; typically an incubation time of 10 minutes is a good starting point and in many cases will be optimal or acceptable; typically washing should be thorough and typically do not use a vortex, but gently agitate, e.g., by hand to re-suspend the solution; typically ensure that the beads are properly into solution before applying the magnet; typically manually pipette the liquid out of the tube, in almost all cases do not vacuum aspirate (aspiration of the beads from a sample tube when discarding supernatant can result in a lack of agent recovery); typically adding additional wash steps can reduce background debris, however it may also decrease the recovery efficiency of an agent; change microcentrifuge tubes between wash steps if background contamination is a concern; and many undesirable compounds will adhere to surfaces (e.g., plastic), so switching the vessel may reduce carry-over.

Others methods for concentrating an agent(s) from or in a sample(s) involve binding of the agent(s) via charge interactions. For example, an agent of interest may exhibit a charge or can be processed to exhibit a charge. Then concentration and purification methods that utilize charge interactions can be utilized. These include, but are not limited to, ion exchange chromatography and Chargeswitch® technology available from Invitrogen, Carlsbad, Calif.

ChargeSwitch® Technology features a charged surface that is “switchable” by changing the pH of the surrounding buffer. At low pH, the surface is positively charged and binds negatively charged agents. Surfaces including microplates are coated with this ChargeSwitch® surface and are available from Invitrogen, Carlsbad, Calif. In some embodiments, ChargeSwitch® Technology is utilized to bind negatively charged agents. In some embodiments, a negatively charged agent is a nucleic acid. For example, at low pH (e.g., about pH 6.5), the surface is positively charged and binds the negatively charged nucleic acid backbone, allowing easy removal of proteins and other contaminants using a simple wash step. Then in some embodiments, the pH is raised (e.g., to about 8.5) to elute the bound agent into the solution. Exact pH levels may vary depending on the agent. These pH levels are known or can be determined easily by one skilled in the art. ChargeSwitch® Technology can be utilized to purify, isolate or concentrate essentially any agent that exhibits a charge or it can be used to remove charged material from a sample away from an agent(s) of interest. In some embodiments, ChargeSwitch® Technology is utilized to isolate, purify, concentrate or remove genomic DNA (e.g., gDNA from plants, bacteria, animal cells, tissues, etc., a plasmid, PCR products, nucleic acids of or from viruses. Numerous related kits, reagents and protocols are available from Invitrogen, Carlsbad, Calif. Chargeswitch technology can also be utilized to isolate agents from or prepared from (e.g., amplified from) essentially any sample including, but not limited to, buccal swabs or forensic sample types, including blood, saliva, hair, semen, cigarette butts, and samples collected from various “touch” surfaces.

In some embodiments, affinity chromatography or related methods are utilized to process, concentrate, purify, or isolate an agent in a sample. This includes column chromatography methods.

Making binding sites on agents accessible to binding molecules can be an important function of a sample preparation. In some embodiments, no treatment is necessary as, for example, when the binding site is an epitope on the surface of a microbe in an aqueous sample that is freely accessible to a binding molecule. In some embodiments, sample preparation is performed that makes an internal binding site of an agent accessible to a binding molecule. This is the case, for example, when nucleic acid binding molecules are used to bind to binding sites on genomic DNA. Target cells are made permeable, such as by lysis, to the probes and their genomic DNA can be denatured. When a large number of different types of agents are tested for in the same sample, the sample preparation is effective for the entire spectrum of targets. In some embodiments, sample preparation includes, but is not limited to, cell lysis.

### Detectable Labels and Detection Methods

Labels useful in the invention described herein include any detectable substance attached or associated with a binding molecule or agent directly or indirectly. Any effective detection method can be used including, but not limited to, optical, fluorescent, light scattering, spectroscopic, electrical, piezoelectrical, magnetic, Raman scattering, surface plasmon resonance, radiographic, calorimetric, and colorimetric methods.

Various approaches for labeling binding molecules can be used in accordance with the present invention, e.g., to achieve a desired sensitivity level. A variety of signal generating labels can be used in accordance with the present invention including, but not limited to, fluorescent dyes, fluorescently dyed nanospheres, polymerized fluorophore molecules, light scattering labels (LSLs), quantum dots, phosphors, lumiphores, fluorophores, chromogens, radioactive isotopes, magnetic particles, metal nanoparticles such as a gold or silver nanoparticles, enzymes, or enzyme-coated particles. These labels can generate a variety of types of signals including, but not limited to fluorescent, chemiluminescent, and colorimetric.

The invention can exploit various types of signal character including: fluorescence, scattered light, light polarization, radio waves, particle size, magnetic field, chemiluminescence, and radioactivity. There can be multiple signal classes within a signal character. For example, if a signal character is fluorescence, various characteristic emission spectra comprise the signal classes (e.g., red fluorescence, green fluorescence, and blue fluorescence). General approaches that can be used with this invention to generate high signal complexity are: (1) distinct labeling, (2) combinatorial labeling, and/or (3) ratio labeling. For examples of general methods for detection, see U.S. Patent Publication Nos. 2003/0170613, 2003/0143580, and 2003/0082516.

Exemplary labels include, but are not limited to, a cyanine, an oxazine, a thiazine, a porphyrin, a phthalocyanine, a fluorescent infrared-emitting polynuclear aromatic hydrocarbon such as a violanthrone, a fluorescent protein, a near IR squaraine dye, a fluorescein, a 6-FAM, a rhodamine, a Texas Red, a tetramethylrhodamine, a carboxyrhodamine, a carboxyrhodamine 6G, a carboxyrhodol, a carboxyrhodamine 110, a Cascade Blue, a Cascade Yellow, a coumarin, Cy2®, Cy3®, Cy3.5®, Cy5®, Cy5.5®, a Cy-Chrome, a phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, a fluorescein isothiocyanate (e.g., fluorescein-5-isothiocyanate), a 5-FAM (5-carboxyfluorescein), a 6-FAM (6-carboxyfluorescein), a 5,6-FAM, a 7-hydroxycoumarin-3-carboxamide, a 6-chloro-7-hydroxycoumarin-3-carboxamide, dichlorotriazinylaminofluorescein, a tetramethylrhodamine-5 (and -6)-isothiocyanate, a 1,3-bis-(2-dialkylamino-5-thienyl)-substituted squarines, the succinimidyl esters of 5 (and 6) carboxyfluoroscein, a 5 (and 6)-carboxytetramethylrhodamine, a fluorescein maleimide, a 7-amino-4-methylcoumarin-3-acetic acid, a 7-amino-4methylcoumarin-3-acetic acid, BODIPY FL, BODIPY FL-Br2, BODIPY 530/550, BODIPY 558/568, BODIPY 564/570, BODIPY 576/589, BODIPY 5811591, BODIPY 630/650, BODIPY 650/665, BODIPY R6G, BODIPY TMR, BODIPY TR, conjugates thereof, and combinations thereof. For more examples, see Dyes and Pigments 17:19-27 (1991) or U.S. Pat. No. 5,631,169. Labels include, but are not limited to organo-metallic complexes such as ruthenium and lanthanide complexes such as described in U.S. Pat. Nos. 4,745,076 and 4,670,572. Exemplary lanthanide chelates include europium chelates, terbium chelates and samarium chelates.

In some embodiments, a label is an enzyme. Exemplary enzymes, which can create a detectable signal in the presence of suitable substrates and assay conditions, include, but are not limited to, alkaline phosphatase, horseradish peroxidase, β-galactosidase, glucose oxidase, galactose oxidase, neuraminidase, a bacterial luciferase, an insect luciferase and a sea pansy luciferase (e.g., Renilia koefiikeri).

Incorporating numerous signal elements can increase the fluorescence intensity of a signaling moiety. For example, fluorescent nanospheres (e.g., about 20 nm in diameter; for example from Invitrogen, Carlsbad, Calif.) can generate a signal equivalent to about 180 fluorescein molecules. Fluorescently dyed polystyrene microparticles (e.g., about 1 nm in diameter) can incorporate millions of fluorophore signaling elements.

A large number of covalent attachment strategies suitable for attaching or associating a label (e.g., a light scattering label (LSL), a quantum dot or a nanocrystal) to a binding molecule are known to those skilled in the art. For example, an amino group can be introduced into a label binding molecule, e.g., through standard synthesis chemistries. Chemistries to activate a label for covalent coupling to an amine-modified or amine containing binding molecule include, but are not limited to, cyanogen bromide, N-hydroxysuccinimide or carbodiimide. Affinity Chromatography by W. H. Scouten, 1981, John Wiley & Sons, and Solid Phase Biochemistry, Analytical And Synthetic Aspects by W. H. Scouten, 1983, John Wiley & Sons) describe activation techniques that can be practices in accordance with the present invention. In some cases, for example N-hydroxysuccinimide and carbodiimide, the label will typically contain at least one surface carboxyl group; for cyanogen bromide activation the label will typically contain at least one surface hydroxyl group. Hetero- and homo-bifunctional linkers might also be employed in such covalent conjugations.

In some embodiments of the invention, detection is via light scattering or RLS. RLS detection methods are described herein. A LSL is a molecule or a material, often a particle, which causes incident light to be scattered elastically, e.g., substantially without absorbing the light energy. LSL particles with the appropriate chemical groups and diameter for use as LSLs can be obtained from several commercial sources (for example, Bangs Laboratories, Inc., Carmel, Ind., USA). Additionally, U.S. Pat. No. 6,586,193 describes methods for labeling binding molecules, e.g., labeling antibodies with an LSL. LSLs are described in detail elsewhere herein.

In the art of material science and related fields, it is known that certain types of molecules can be attached to surfaces, other molecules or metals and the like. Typically, there are certain types of chemical groups at specific locations within a molecule which allow for one part of the molecule to become bound to a surface, a second molecule or a label, e.g., while other parts are not bound to the surface. For example, the adsorption of thiol and disulfide containing substances, and amphiphilic substances, such as n-alkonic acids and certain detergent molecules onto metal surfaces is known (e.g., see Nuzzo et al., Journal of the American Chemical Society, 105:4481-4483 (1983); Allara et al., Langmuir 1:45-52 (1984); and Bain et al., i Journal of the American Chemical Society, 111:321-335 (1989). In some embodiments, attachment is conferred onto binding molecule and other substances by incorporating an appropriate chemical group(s) into location(s) within the molecular structure of the substance that is to be attached and/or into the binding molecule. In some embodiments, molecules are attached whose molecular structure is charged or ionic, or is polarized such that at one end of the molecular structure it is hydrophobic while at the other end it is hydrophilic. For further methods of attaching various labels, see U.S. Pat. No. 5,294,369.

In some embodiments, nucleic acids containing a phosphate backbone which contains a high negative charge are labeled, e.g., with a metal particle. In some embodiments, a single-stranded nucleic acid is end labeled with a thiol or disulfide at the 3′ or 5′ end with or without additional hydrophobic groups incorporated into the same region of the molecule. This modified nucleic acid will bind to the metal surface or particle at the end labeled with these groups. The ionic part of the nucleic acid keeps the main chain of the nucleic acid's molecular structure away from the surface such that it is accessible for molecular interactions with most any substance that can specifically bind to it. Other types of molecules can be similarly attached to metal particles.

Linker arms of various lengths and composition can also be incorporated into a molecular structure. For example, a molecule can be used where its molecular structure is optimized for attachment, for example to a label or binding molecule. As an example, a polypeptide can be chemically modified (e.g., at one terminus) by the addition of a disulfide or a thiol chemical group(s). The polypeptide may be composed of amino acids or engineered such that the polypeptide chain will not significantly interact with a label or binding molecule except through the chemically modified portion. At the other terminus a free amino group exists, or alternatively, has been chemically modified for a desired conjugation process such that a desired substance can be attached at this position.

One of ordinary skill in the art will recognize the many different variations of attachment methods that can made by varying the chemical groups, molecular weights, molecular structure, labeling reaction conditions, and the type of conjugation chemistry (e.g., cross-linking, covalent attachment, etc.) that is used.

Some embodiments of the invention include an ability to use large area imaging to detect individual targets. Detection of agents labeled (e.g., directly or indirectly) with signaling moieties can be effected once the complexes are localized, e.g., in a detection zone. The detection process used depends on the type of signal character of the signaling moieties (e.g., fluorescence, chemiluminescence, or light scattering). For some signal characters (e.g., light scattering and fluorescence), complexes, e.g., in a detection zone, are illuminated by a light source. For others (e.g., chemiluminescence, radio transmission, or magnetic fields), illumination may not be required. Various detection modes can be used including CCD cameras, film, and direct visualization.

Various imaging systems can be used to detect and analyze signals from semiconductor nanocrystals. In some embodiments, an imaging system (e.g., automated detection) for use with the present methods comprises an excitation source, optionally a monochromator (or any device capable of spectrally resolving the image, or a set of narrow band filters) and a detector array. In some embodiments, an excitation source can comprise blue or UV wavelengths shorter than the emission wavelength(s) to be detected. This may be, but is not limited to, a broadband UV light source, such as a deuterium lamp, e.g., with a filter in front; an output of a white light source such as a xenon lamp or a deuterium lamp (e.g., after passing through a monochromator to extract out the desired wavelengths); or any of a number of continuous wave (cw) gas lasers, including but not limited, to any of the Argon Ion laser lines (457, 488, 514, etc. nm) or a HeCd laser; a solid state diode laser in the blue such as GaN and GaAs (doubled) based lasers or the doubled or tripled output of YAG or YLF based lasers; or any of the pulsed lasers with output in the blue.

In some embodiments, emitted light can be detected with a device that provides spectral information for the substrate, e.g., a grating spectrometer, a prism spectrometer, an imaging spectrometer, or the like, or use of interference (bandpass) filters. In some embodiments, a two-dimensional area imager such as a CCD camera, is used to image many objects simultaneously. In some embodiments, spectral information is generated by collecting more than one image, e.g., via different bandpass, longpass, or shortpass filters (e.g., interference filters, or electronically tunable filters as appropriate). In some embodiments, more than one imager may be used to gather data simultaneously e.g., through dedicated filters, or the filter may be changed in front of a single imager. In some embodiments, an imaging based systems is utilized that can scan a surface to find fluorescent signals such as a biometric imaging system.

When imaging samples labeled with multiple fluorophores, it is desirable to resolve spectrally the fluorescence, e.g., from each situs. Such samples can arise, for example, from multiple types of semiconductor nanocrystals (and/or other fluorophores) being used or from multiple molecules labeled with different types of fluorophores bound at a single location. Decoding the spectral code of a sample can take place prior to, simultaneously with, or subsequent to determining whether a label is associated with an agent.

Techniques related to imaging include, but are not limited to, Fourier transform spectral imaging (Malik et al., J. Microsc. 182:133 (1996); Brenan et al., Appl. Opt. 33:7520 (1994)) and Hadamard transform spectral imaging (Treado et al., Anal. Chem. 61: 732A (1989); Treado et al., Appl. Spectrosc. 44:1-4 (1990); Treado et al., Appl. Spectrosc. 44:1270 (1990); Hammaker et al., J. Mol. Struct. 348;135 (1995); Mei et al., J. Anal. Chem. 354:250 (1996); and Flateley et al., Appl. Spectrosc. 47:1464 (1993)), imaging through variable interference (Youvan, Nature 369:79 (1994); Goldman et al., Biotechnology 10:1557 (1992)), acousto-optical (Mortensen et al., IEEE Trans. Inst. Meas. 45:394 (1996); Turner et al., Appl. Spectrosc. 50:277 (1996)) or liquid crystal filters (Morris et al., Appl. Spectrosc. 48:857 (1994)) or simply scanning a slit or point across the sample surface (Colarusso et al., Appl. Spectrosc. 52:106A (1998)), many of which are capable of generating spectral and spatial information across a two-dimensional region of a sample.

### Binding Molecules

The term “binding molecule” refers to any molecule that can bind or attach to another molecule or agent of interest. For example, a molecule (e.g., an antibody) that binds an agent of interest would be considered a binding molecule. Binding molecules include, but are not limited to, lectins or fragments (or derivatives) thereof which retain binding function; antibodies (e.g., monoclonal, including chimeric or genetically modified monoclonal antibodies, humanized, or polyclonal)); peptides; aptamers; nucleobases (synthetic, natural, or modified); nucleic acid molecules (including, but not limited to, single stranded RNA or single-stranded DNA, or single-stranded nucleic acid hybrids); biotin; avidin, or streptavidin, or avidin derivatives; a transcription factor; or a Zinc finger binding protein. In some embodiments, a binding molecule(s) is from the U.S Government's Critical Reagents Program which is a repository of reagents made available to groups working under U.S. Government contract. For clarity, either member of a binding pair is considered a binding molecule. In some instances, one member of a binding pair can be an agent.

The term “capture binding molecule” refers to a binding molecule that initially binds the agent in an assay. In some embodiments, a capture binding molecule is immobilized, e.g., to a reactive surface. Binding molecules will typically bind through non-covalent interactions such as ionic attractions, hydrogen bonding, Vanderwaals forces, hydrophobic interactions and the like. Although, in some embodiments, binding may be through covalent interactions. Typical interactions of binding molecules include, by way of example and not limitation: immunological interactions between an antibody or Fab fragment and its antigen, hapten or epitope; biochemical interactions between a protein (e.g. hormone or enzyme) and its receptor (for example, avidin or streptavidin and biotin), or between a carbohydrate and a lectin; chemical interactions, such as between a metal and a chelating agent; nucleic acid base pairing between complementary nucleic acid strands and between a peptide nucleic acid analog (PNA) and a corresponding nucleic acid. In some embodiments, a capture binding molecule is an antibody that binds an agent, e.g., wherein the agent is a protein or peptide from an infectious agent. This embodiment can be used for example, to detect a viral agent in a sample. In some embodiments, a capture binding molecule is a protein or peptide from an infectious agent that binds an antibody(s), in this case the antibody is the agent to be detected. This embodiment can be utilized to measure the level of antibodies in serum for a particular infectious pathogen(s). In some embodiments of the invention, one or more capture binding molecules are first immobilized onto a surface, e.g., of an optical waveguide to form a reactive surface.

Essentially any type of antibody may be utilized as a binding molecule in accordance with the present invention. These include, but are not limited to, synthetic antibodies, monoclonal antibodies, recombinantly produced antibodies, intrabodies, multispecific antibodies, bispecific antibodies, human antibodies, humanized antibodies, chimeric antibodies, synthetic antibodies, single-chain Fvs (scFv), Fab fragments, F(ab′) fragments, disulfide-linked Fvs (sdFv), anti-idiotypic (anti-Id) antibodies, and epitope-binding fragments of any of the above. Antibodies used in the methods of the present invention include immunoglobulin molecules and immunologically active portions of immunoglobulin molecules. The immunoglobulin molecules of the invention can be essentially of any type (e.g., IgG, IgE, IgM, IgD, IgA and IgY), class (e.g., IgG1, IgG2, IgG3, IgG4, IgA1 and IgA2) or subclass of immunoglobulin molecule.

Antibodies or antibody fragments can be essentially from or derived from any organism including, but not limited to, a bird, a mammal, a mouse, a human, a goat, a bovine, a donkey, a guinea pig, a camel, a chicken, a sheep, a dog, a cat, a horse, a rat, a hamster or a rabbit. In some embodiments, the antibodies are human or humanized antibodies, e.g., monoclonal. As used herein, “human” antibodies include antibodies having the amino acid sequence of a human immunoglobulin and include antibodies isolated from human immunoglobulin libraries or from animals (e.g., a mouse) that express antibodies from human genes. In some embodiments, an antibody is a murine antibody. Antibodies or antibody fragments used in accordance with the present invention may be monospecific, bispecific, trispecific or of greater multispecificity. Multispecific antibodies may specifically bind to different epitopes of a desired target molecule or may specifically bind to both a target molecule as well as a heterologous epitope, such as a heterologous polypeptide or solid support material. See, e.g., PCT Publication Nos. WO 93/17715, WO 92/08802, WO 91/00360, and WO 92/05793; U.S. Pat. Nos. 4,474,893, 4,714,681, 4,925,648, 5,573,920, and 5,601,819; Tutt et al., J. Immunol. 147:60-69 (1991); and Kostelny et al., J. Immunol. 148:1547-1553 (1992). The present invention may also be practiced with single domain antibodies, including camelized single domain antibodies (see e.g., Muyldermans et al., Trends Biochem. Sci. 26:230 (2001); Nuttall et al., Cur. Pharm. Biotech. 1:253 (2000); Reichmann and Muyldermans, J. Immunol. Meth. 231:25 (1999); PCT Publication Nos. WO 94/04678 and WO 94/25591; and U.S. Pat. No. 6,005,079). In some embodiments, an antibody is a human antibody. In some embodiments, an antibody is a humanized antibody.

Well known techniques are available for the preparation of an antibody(s) or an antibody fragment for binding a particular agent(s) and need not be described in detail. For example, an animal is immunized or challenged with a desired antigen/agent according to an appropriate immunization schedule. In some cases, the antigen/agent is coupled to a carrier molecule such as BSA to improve recognition. In some embodiments, the immunization is performed with an adjuvant. After a suitable time period, the animal is bled and antibodies are extracted. Alternatively, antibody can be obtained from ascites fluid. Other methods for generating desired antibodies are known in the art, e.g., utilizing display techniques such as phage display (e.g., see U.S. Pat. No. 7,118,879). Antibodies have numerous amino, carboxyl and sulfhydryl groups that might be utilized for coupling reactions.

The construction of chimeric antibodies is a procedure in which a chimeric antibody is made by, e.g., joining a murine variable region to a human constant region. Additionally, “humanized” antibodies may be made by joining a hypervariable region of an antibody (e.g., murine) to a constant region and portions of variable region (light chain and heavy chain) sequences of human immunoglobulins using one of several techniques known in the art.

Aptamers can be made using methods known in the art, e.g., described in U.S. Pat. No. 5,789,157. Lectins, and fragments thereof, are also commercially available.

Synthesis of binding molecules comprised of oligonucleotides is also routine, using automated synthesizers such as the ABI 480. These instruments prepare oligonucleotides of virtually any desired sequence. In some embodiments, oligonucleotides can be modified with terminal amines or other reactive groups for coupling. A review of coupling chemistries is found in Goodchild, Bioconjugate Chemistry, 1(3):165-187 (1990).

In some embodiments, a binding molecule is covalently attached to a reactive surface, e.g., through chemical coupling means. In some embodiments, a reactive surface may be derivatized directly with a variety of chemically reactive groups which then, under certain conditions, form stable covalent bonds with the applied binding molecule. Alternatively, a reactive surface may first be coated with chemically-derivatized polymers, such as dextran or PEG, which then form covalent bonds with applied binding molecules. Certain types of detergents may also be coated to the reactive surface, then derivatized, in situ, and reacted with binding molecules. For example, glass and quartz waveguides contain groups that can be activated to reactive hydroxyl and siloxy groups, which can be coupled to specific binding molecules via linkers. Such linkers include, for example, homo- and hetero-bifunctional linkers. In some embodiments, a reactive surface is glass treated with 3-aminopropyltriethoxysilane.

Typically, a label is covalently bound to a binding molecule, but this is not essential. Physical adsorption of a binding molecule(s) onto labels or vice versa is also suitable. The attachment need only be strong enough to withstand the subsequent reaction conditions without substantial loss of the label, e.g., from washing steps, other fluid flow or other steps of the assay.

Typically, a binding molecule when associated with a reactive surface is done in such a manner that the specific binding properties of a binding member are not lost. For example, an antibody can be coupled via its Fc portion (e.g., see U.S. Pat. No. 5,191,066) and oligonucleotides can be coupled via terminal amines or other functional groups. Linker arms (e.g., see U.S. Pat. No. 4,948,882) can be placed on “sterically tolerant” positions of base moieties to facilitate coupling to solid phases without loss of hybridization or binding capabilities. In some embodiments, a reactive surface may be coated with a binding molecule such as streptavidin through physical adsorption and then reacted with a biotin-labeled binding molecule or vice versa with biotin coating and a streptavidin-labeled binding molecule. However, for various embodiments of the invention a binding molecule associated with a surface need not be covalently attached.

### Light Scattering (LS)

Some embodiments of the invention utilize LS such as resonance light scattering (RLS) as a detection method or detection means. Therefore, the present invention provides methods, inter alia, for detecting, analyzing, quantitating or identifying an agent using LS. In some embodiments, LS is performed using LS particles as labels. In some embodiments of the invention, the detection and/or measurement of the light-scattering properties of the particles typically correlate to the presence and/or amount, or absence, of one or more analytes or agents in a sample. Various aspects and descriptions related to light scatter and related detection methods are described in, for example, U.S. Pat. Nos. 4,313,734, 4,480,042, 5,017,009, 5,151,956, 5,350,697, 5,599,668, 6,214,560, 6,180,415 and 6,586,193; U.S. Patent Publication Nos. 2001/0002315 and 2002/028519; PCT Publication Nos. WO 97/40181, WO 03/021853 and WO 99/20789; Lin et al., Clin. Diag. Lab. Immunol. 12:418 (2005); Stimpson et al., Proc. Natl. Acad. Sci. USA 92:6379-6383 (1995); Yguerabide & Yguerabide, Anal. Biochem. 261:157-176 (1998); Yguerabide & Yguerabide Anal. Biochem. 262:137-156, (1998); Yguerabide & Yguerabide, Journal of Cellular Biochemistry Supplement 37:71-81 (2001); Schultz et al., Proc. Natl. Acad. Sci. 97:996-1-1 (2000); Bao et al., Analytical Chemistry 74:1792-1797 (2002); Absorption and Scattering of Light By Small Particles Bohren et al., John Wiley and Sons (1983); The Scattering of Light and Other Electromagnetic Radiation, Kerker, Academic Press (1969); Colloids and the Ultramicroscope-A Manual of Colloid Chemistry and Ultramicroscopy, Zsigmondy, John Wiley & Sons, Inc (1914); Hunter, Foundation of Colloid Science, Vol. I:105 (1991); Shaw et al., Introduction to Colloid and Surface Chemistry, 2nd ed., 41, 1970; Stolz, SpringerTracts, Vol. 130; Klein and Metz, Photographic Science and Engineering 5:5-11, (1961); Eversole and Broida, Physical Review 15:1644-1654, (1977); Kreibig and Zacharias, Z. Physik 231:128-143 (1970); Bloemer et al., Physical Review 37:8015-8021 (1988); Wiegel, Zeitschrift fur Physik, Bd. 136:642-653 (1954); Hayat, “Immunogold-Silver Staining”, CRC Press, Inc. (1995); and “GeniconRLS™ One-Color and Two-Color Microarray Toolkits™” Instruction Manual, Version B, Jan. 18, 2005 from Invitrogen (Carlsbad, Calif.).

In general LS technology is based on physical properties of particles (e.g., metal colloidal particles). In some embodiments, these particles are nanometer-sized and, when illuminated with either coherent or polychromatic light, the particles scatter incident radiation in a manner consistent with electromagnetic theory known as resonance light scattering. The light produced by sub-microscopic RLS particles arises when their electrons oscillate in phase with incident electromagnetic radiation, although applicants do not wish to be bound by any theoretical speculation as to the mechanistic explanation. The resulting scattered light is typically in the visible range and typically intense, often being at least several orders of magnitude greater than fluorescence light when compared on a per label basis. The level of intensity and color is determined largely by particle composition, size and shape. Typically when illuminated with white light, a particle suspension preferentially scatters light that has a color that corresponds to the peak wavelength of its light scattering spectral band (e.g., see Yguerabide and Yguerabide, Analytical Biochemistry 262:137-15 (1998) and Yguerabide and Yguerabide, Analytical Biochemistry 262:157-176 (1998)).

LS technology can be used in methods which detect low concentrations of agents, and in some cases, without the need for signal or agent molecule amplification. In some embodiments, LS allows for the detection of agents wherein the amount and types of reagents are typically reduced relative to some other methods in the art. In some embodiments, using LS technology, they typically require about 10-fold less starting material than fluorescence methods.

In contrast to the use of fluorescent labels, where the agent(s) binds to a compound comprising a fluorescent molecule, the principle behind LS is that the agent(s) is bound to at least one detectable light scattering particle (directly or indirectly). In some embodiments, a LS particle has a size smaller than the wavelength of the illuminating light. These particles are illuminated with a light beam under conditions where the light scattered by the particle can be detected. The scattered light is then a measure of the presence of one or more agents in a sample.

Some benefits of LS particles include that many of these particles they do not photobleach, fade, quench or decay; the color or wavelength of the scattered light can be changed by altering particle composition and/or particle size; and/or the particles can be coated with binding molecules (e.g., antibodies or nucleic acid probes) for detection of agents including analyte antigens or DNA sequences. Furthermore, LS particles often offer a broad dynamic range: by judicious choice of integrated light intensity measurements or direct observation by eye, an agent can be detected over a wide range of agent concentrations, and the region of dynamic range can be adjusted by changing the particle size. LS particles are also often compatible with homogeneous assays, for example in solution, or in solid phase assays wherein high sensitivity can be obtained through particle counting. In short, LS often allows sensitive quantitative assays can be conducted with relatively simple instrumentation.

To affect specific binding in analytical bioassays, the surface of LS particles can be derivatized with a variety of biomolecules/or binding molecules. For related methods and disclosure see Yguerabide and Yguerabide, Analytical Biochemistry 262:137-15 (1998) and Yguerabide and Yguerabide, Analytical Biochemistry 262:157-176 (1998).

The wide range of specific light scattering signals from different particle types means that one skilled in the art typically can detect and measure to a high degree of specificity one or more analytes or agents in a sample. Some embodiments of the invention utilize optical resolvability of two or more different particle types for multi-agent detection, e.g., simultaneous detection of two or more different agents in a sample. In some embodiments, the use of specific particle types that possess measurable and detectable light scattering properties in a defined assay format enables ready application of methods described herein to micro-arrays and other high-throughput techniques. The color and intensity of the scattered light signal is typically a function of particle size, shape and composition.

In many instances, modest increases in gold particle size results in a relatively large increase in the light scattering power of the particle (the Csca). The incident wavelength for the maximum Csca is increased significantly with particle size and the magnitude of scattered light intensity is significantly increased. When illuminated with white light, certain metal-like particles of identical composition but different size can be distinguished from one another in the same sample by the color or wavelength of the scattered light. The relative magnitude of the scattered light intensity can be measured and used together with the color or wavelength of the scattered light to detect different particles in the same sample specifically and sensitively, and in some instances, even in samples with high non-specific light backgrounds. In some embodiments, LSLs utilized in the present invention are colloidal particles, such as colloidal gold, silver or selenium or minute latex particles.

In some embodiments, the LS particles (e.g., gold or silver particles) are or have an average diameter of about 1, about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 1000, or about 10,000 nm. In some embodiments, LS particles (e.g., gold or silver particles) are or have an average diameter from about 1 nm to about 10,000 nm, from about 1 nm to about 1000 nm, from about 1 nm to about 100 nm, from about 1 nm to about 75 nm, from about 1 nm to about 50 nm, from about 1 nm to about 25 nm, from about 25 nm to about 75 nm, from about 25 nm to about 50 nm, from about 50 nm to about 75 nm, from about 35 nm to about 45 nm, from about 40 nm to about 50 nm, from about 45 nm to about 55 nm, from about 50 nm to about 60 nm, from about 55 nm to about 65 nm, from about 60 nm to about 70 nm, from about 65 nm to about 75 nm, from about 70 nm to about 80 nm, from about 75 nm to about 85 nm, from about 80 nm to about 90 nm, from about 85 nm to about 95 nm, from about 90 nm to about 100 nm, from about 95 nm to about 105 nm, from about 100 nm to about 110 nm, from about 105 nm to about 115 nm, from about 110 nm to about 120 nm, from about 115 nm to about 125 nm, from about 100 nm to about 200 nm, from about 200 nm to about 300 nm, from about 300 nm to about 400 nm, from about 400 nm to about 500 nm, from about 500 nm to about 600 nm, from about 600 nm to about 700 nm, from about 700 nm to about 800 nm, from about 800 nm to about 900 nm, from about 900 nm to about 1000 nm, from about 1000 nm to about 2500 nm, from about 2500 nm to about 5000 nm, from about 5000 nm to about 7500 nm, from about 7500 nm to about 10000 nm, from about 37.5 nm to about 42.5 nm, from about 42.5 nm to about 47.5 nm, from about 47.5 nm to about 52.5 nm, from about 52.5 nm to about 57.5 nm, from about 57.5 nm to about 62.5 nm, from about 62.5 nm to about 67.5 nm, from about 67.5 nm to about 72.5 nm, from about 72.5 nm to about 77.5 nm, from about 67.5 nm to about 72.5 nm, from about 72.5 nm to about 77.5 nm, from about 77.5 nm to about 82.5 nm, from about 82.5 nm to about 87.5 nm, from about 87.5 nm to about 92.5.5 nm, from about 92.5 nm to about 97.5 nm, or from about 97.5 nm to about 102.5 nm.

Commercially available particle preparations typically have particle size distributions e.g., from about <10 to about <20 percent coefficient of variation. Percent coefficient of variation is defined as the standard deviation of the particle size distribution divided by the mean of the particle preparation. For example, for a 60 nm particle preparation with a coefficient of variation of 20%, one standard deviation unit is about ±12 nm. This means that about 10% of the particles are smaller than 48 nm or greater than 72 nm. Such variation in size can have effects on the intensity of scattered light and the color of scattered light depending on the approximate “mean” size of the particles in the preparation.

In some embodiments of the invention, label particles have size distribution with a coefficient of variation between from about 0.1% to about 40%, from about 1% to about 30%, from about 0.1% to about 10%, from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 2.5% to about 7.5%, from about 7.5% to about 12.5%, from about 12.5% to about 17.5%, from about 17.5% to about 22.5%, from about 22.5% to about 27.5%, from about 27.5% to about 32.5%, from about 32.5% to about 37.5%, or from about 37.5% to about 42.5%.

The labeling particles utilized in some embodiments of the present invention can be of various shapes. In some embodiments, labeling particles used in an assay are of an essentially homogeneous shape. In some embodiments, labeling particles used in an assay are of more than one shape. Shapes of labeling particles (e.g., LSLs) can be, but are not limited to, spherical, oval, ellipsoidal, asymmetrical, rods, stars or multi-particle aggregates.

In some embodiments, it is also possible to utilize a LAM in the solution, e.g., in contact with the reaction surface. This has the advantage of reducing background scattering very near to its source. LAMs are described in detail elsewhere herein.

In some embodiments, the labeling particles for RLS or light scattering are comprised of gold, silver, copper, aluminum, latex, selenium, polystyrene, polymethylacrylate, polycarbonate or similar materials. In the case of metals, the particles can also be salts of a metal(s). In some embodiments, the particles comprise at least two different elements, e.g., gold and silver or silver plated gold particles. In some embodiments, light scattering particles are comprised of 2, 3, 4, 5, 6, 7, 8, 9, 10 or more elements. In some embodiments, light scattering particles are comprised of between from about 1 to 20 elements; from about 1 to 10 elements; from about 10 to 20 elements; from about 1 to 5 elements; from about 5 to 10 elements; from about 10 to 15 elements; from about 15 to 20 elements; from about 2 to 4 elements; from about 4 to 6 elements; from about 6 to 8 elements; from about 8 to 10 elements; or from about 10 to 12 elements. Light scattering particles/labels can be produced by methods known in the art or can be purchased from commercial entities, e.g., Bangs Laboratories, Inc., Fishers, Ind. or BioAssay Works, Ijamsville, Md.

In some embodiments, at least two different LSLs can be employed in the same assay, e.g., to detect at least two different agents. The at least two different LSLs can vary based on their composition (e.g., one comprises gold and the other silver), based on their size (e.g., one is about 80 nm and the other is 60 nm), based on both their composition and size (e.g., 80 nm gold particles and 60 nm silver particles), based on their shape, or combinations thereof. In some embodiments, 2, 3, 4, 5, 6, 7, 8, 9, 10, 11, 12, or more different LSLs are employed in the same assay. In some embodiments, between from about 2 to about 12, from about 2 to about 4, from about 2 to about 8, from about 2 to about 10, from about 10 to about 12, from about 8 to about 12, from about 6 to about 12, from about 4 to about 12, from about 2 to about 10, from about 4 to about 8, from about 4 to about 6, or from about 6 to about 8, different LSLs are employed in the same assay.

In some embodiments, a combination of LSLs and at least one other label type (e.g., a fluorophore) are utilized in an assay. This can be used to, inter alia, detect multiple agents.

Some embodiments of the invention provide a signal generation and detection system including a control and analysis system, a signal generation and detection apparatus, or reader, and companion software for controlling the reader and for capturing, processing and analyzing LS images and other data. In some embodiments, a reader includes an illumination system having a shutter/aperture assembly for delivering precise patterns of light to a sample and a detection system comprising a camera (e.g., a charge-coupled device (CCD) camera). The system may be operated manually or via software instructions and algorithms for generating, capturing, processing and analyzing images (e.g., RLS images). In some embodiments, the control system performs multiplexed assays of two or more colors or wavelengths, e.g., to allow separation and analysis of detected light from labels.

Typically, a label is covalently bound to a binding molecule, but this is not essential. Physical adsorption of a binding molecule(s) onto particulate labels (e.g., light scattering labels) is also suitable. The attachment need only be strong enough to withstand the subsequent reaction conditions without substantial loss of the label, e.g., from washing steps, other fluid flow or other steps of the assay.

Various methods for attaching or associating a label with a binding molecule are known in the art or described herein. Additionally, there are companies that will provide as service the labeling of binding molecules, e.g., BioAssay Works, Ijamsville, Md., provides labeling of compounds such as binding molecules with metal particles.

### Light Absorbing Materials

In some embodiments of the invention, a light absorbing material (LAM) may be utilized in accordance with an assay of the present invention. In some embodiments, a LAM is added to a mixture of sample and labeled binding molecule. A LAM can be designed to prevent stray light from interfering in a light scattering reaction. Without being bound by theory, it is believed that stray light arises primarily from microscopic imperfections in the reflecting interface and from scattering of the evanescent wave by particles that migrate to, but are not bound in, the penetration depth. A LAM can be designed such that, when dispersed in bulk solution, it absorbs and minimizes the effect of such stray light, typically better than when such a material is coated onto a surface to form an opaque layer. Suitable LAMs include the conjugate itself as well as numerous light absorbing compounds or dyes. Light absorbing dyes are any compounds that absorb energy from the electromagnetic spectrum, ideally at wavelength(s) that correspond to the wavelength(s) of the light source. In some embodiments, a LAM will be comprised of conjugated heterocyclic structures. In some embodiments, a LAM is selected from, but not limited to, azo dyes, diazo dyes, triazine dyes, food colorings, biological stains, Coomasie Brilliant Blue R-250 Dye (Biorad Labs, Richmond, Calif.); Reactive Red 2 (Sigma Chemical Company, St. Louis, Mo.), bromophenol blue (Sigma); xylene cyanol (Sigma); and phenolphthalein (Sigma). Combinations of essentially any LAMs can be utilized in assays. The Sigma-Aldrich Handbook of Stains, Dyes and Indicators by Floyd J. Green, published by Aldrich Chemical Company, Inc., (Milwaukee, Wis.) provides numerous other dyes and corresponding data. With these data, dyes with the appropriate light absorption properties can be selected to coincide with the wavelengths emitted by the light source.

In most cases, LAMs are selected that do not interfere or do not irreparably interfere with the absorption of a labeled binding molecule (e.g., labeled with a LSL), or with the specificity of a binding molecule of the assay (e.g., an immobilized or labeled binding molecule). For example, if a label binding molecule is a peptide, polypeptide or protein, the LAM typically would not denature the peptide, polypeptide or protein. Similarly, if a labeled binding molecule is a nucleotide sequence, the LAM typically would not denature the nucleotide sequence. Once selected on the basis of light absorption properties, the dyes can be evaluated empirically to ensure the dye does not interfere with the specific binding events required for the particular assay employed.

In some embodiments, a labeled binding molecule or conjugate itself can also serve as a LAM. Using higher than necessary concentrations of a labeled binding molecule or conjugate, for example, concentrations that provide an effective O.D, which in some cases, may be of at least 15, more than 300, or more than 500. In some embodiments, an O.D. is

Methods of concentrating a binding molecule or conjugate include, but are not limited to, affinity purification, filtration, centrifugation, or as described herein for concentrating an agent in a sample. In some embodiments, a LAM dye(s) is used and optionally in conjunction with a concentrated labeled binding molecule or conjugate.

In some cases, a LAM will increase the optical density (O.D.) of the solution, e.g., to at least 15, and provide a dark background against which scattering at the sites shows as a bright area. In some embodiments, a LAM containing solution will be of an O.D. between from about 1 to about 500, about 1 to about 300, about 2 to about 100, about 2 to about 50, about 15 to about 50, about 50 to about 100, about 100 to about 200, about 200 to about 300, about 30 to about 50, about 10 to about 20, about 2 to about 20, from about 2 to about 4, from about 3 to about 5, from about 4 to about 6, from about 5 to about 7, from about 6 to about 8, from about 7 to about 9, from about 8 to about 10, from about 9 to about 11, from about 10 to about 12, from about 11 to about 13, from about 12 to about 24, from about 13 to about 15, from about 14 to about 16, from about 15 to about 17, from about 16 to about 18, from about 17 to about 19, or from about 18 to about 20.

While LAMs are an optional feature of the invention, in some embodiments, their use results in the ability to use higher concentrations of labeled binding molecules or conjugate, higher intensities of light and/or larger label particles, all of which can greatly improve performance. Not wishing to be bound by theory, an enhanced effect of using a LAM is possibly due to the elimination of stray light at a point close to its source. Therefore, the present invention provides compositions comprising a LAM and optionally the composition comprises a labeled binding molecule. The present invention also provides methods of decreasing background signal in an assay or assay chamber of the invention.

### Evanescent Waveguides and Related Methods

Total internal reflection (TIR) is known in the art, e.g., see U.S. Pat. Nos. 4,608,344; 5,192,502; and 5,599,668. Total internal reflection is an optical phenomenon that occurs when light strikes a medium boundary at a “steep” angle. If the refractive index is lower on the other side of the boundary essentially no light can pass through, so essentially all of the light is reflected. The critical angle is the angle of incidence above which the total internal reflection occurs.

When light crosses a boundary between materials with different refractive indices, the light beam will be partially refracted at the boundary surface, and partially reflected. However, if the angle of incidence is shallower (closer to the boundary) than the critical angle, then the light will stop crossing the boundary altogether and instead essentially reflects back internally.

TIR operates upon the principle that light traveling in a denser medium (i.e. having the higher refractive index, N1) and striking the interface between the denser medium and a rarer medium (i.e. having the lower refractive index, N2) is totally reflected within the denser medium if it strikes the interface at an angle, θ_{R}, greater than the critical angle, θ_{C}, where the critical angle is defined by the equation:

θ_{0 C}=arcsin(N₂/N₁)

Under these conditions, an electromagnetic waveform known as an “evanescent wave” is generated. The electric field associated with the light in the denser medium forms a standing sinusoidal wave normal to the interface. The evanescent wave penetrates into the rarer medium, but its energy E dissipates exponentially as a function of distance Z from the interface. A parameter known as “penetration depth” (dₚ) is defined as the distance from the interface at which the evanescent wave energy has fallen to 0.368 times the energy value at the interface. See, Sutherland et al., J. Immunol. Meth., 74:253-265 (1984) defining dₚ as the depth where E=(e⁻¹)·E₀. Penetration depth is calculated as follows:

$${d}_{p} = \frac{λ / {N}_{1}}{2 π {\{ {sin}^{2} {θ}_{R} - {( {N}_{2} / {N}_{1} )}^{2} \}}^{1 / 2}}$$

Factors that tend to increase the penetration depth are: increasing angle of incidence, θ_{R}; closely matching indices of refraction of the two media (e.g., N₂/N₁→1); and increasing wavelength, λ. For example, if a quartz TIR element (N₁=1.46) is placed in an aqueous medium (N₂=1.34), the critical angle, θ_{C}, is 66° (=arcsin 0.9178). If 500 nm light impacts the interface at θ_{R}=70° (i.e. greater than the critical angle) the dₚ is approximately 270 nm.

Within the penetration depth, the evanescent wave in the rarer medium (typically a reaction solution) can excite fluorescence in the sample. Examples of devices and methods related to TIR fluorescence for immunoassays are described, for example, in Harrick, et al., Anal. Chem., 45:687 (1973); U.S. Pat. Nos. 4,447,564, 4,577,109, 4,582,809, 4,654,532, and 4,716,121; and PCT Publication No. WO 93/20240.

Some embodiments of the invention provide methods, assays, and compositions that utilize TIR for the analysis, detection, identification or quantitation of an agent(s) in a sample.

TIR has also been used in conjunction with light scattering detection in a technique referred to as Scattered Total Internal Reflectance (“STIR”). See, e.g., U.S. Pat. Nos. 4,979,821 and 5,017,009 and WO 94/00763. According to this technique, a beam of light is scanned across the surface of a TIR element at a suitable angle and the light energy is totally reflected except for the evanescent wave. Particles such as red blood cells, colloidal gold or latex specifically bound within the penetration depth will scatter the light and the scattered light is detected, e.g., by a photodetection means. Some embodiments of the invention involve scanning the light beam across several loci of specific binding molecules which are either (1) the same binding molecules at varying concentration to achieve a wider dynamic range, or (2) different binding molecules to test for different agents in a multiplex format.

In some embodiments of the invention, devices of the invention are used by sequentially directing a light beam to individual sites and creating small loci of evanescent wave generation. In some embodiments, the entire waveguide is illuminated at once, thereby creating evanescent wave energy across essentially the entire reactive surface. This simultaneous illumination of the entire reactive surface enables simultaneous examination and comparison of all the sites, which can permit a rapid detection method. In some embodiments, the entire waveguide reactive surface can be seen (and/or detected) at once and it is all illuminated simultaneously, so the accumulation of LSL at a situs or region can be observed and compared to other sites in real time since there is no need to scan each situs either for illumination with incident light or for detection of scattered light.

Typically in assays utilizing an evanescent wave method, the reagents and the sample (e.g., conjugate-sample solution), need not be washed off the capture site to allow detection.

### Nanocrystals and Quantum Dots

Some embodiments of the invention provide methods, assays, and compositions that utilize nanocrystals for the analysis, detection, identification or quantitation of an agent(s) in a sample. Some embodiments of the invention utilize nanocrystals as detectable labels. Each of the characteristics of nanocrystals as described herein is examples of characteristics that can be used in accordance with the present invention.

Some characteristics of nanocrystals include that they can be produced in a narrow size distribution and, since the spectral characteristics are a function of the size, can be excited to emit a discrete fluorescence peak of narrow bandwidth. In other words, the ability to control the spectral characteristics of nanocrystals (e.g., narrow bandwidth, discrete emission wavelengths, a single wavelength can excite an array of nanocrystals with different emissions) are some of the major advantages for their use. Another advantage of the nanocrystals is their resistance toward photobleaching under light sources. As known in the art, a manual batch method may be used to prepare semiconductor nanocrystals of relative monodispersity (e.g., the diameter of the core varying approximately 10% between quantum dots in a preparation; e.g., see Bawendi et al., J. Am. Chem. Soc. 115:8706 (1993)).

The term “semiconductor nanocrystal” and “quantum dot” are used interchangeably herein and refer to an inorganic crystallite of about 1 nm or more and about 1000 nm or less in diameter or any integer or fraction of an integer there between.

Semiconductor nanocrystals are quantum dots that can be excited, e.g., with a single excitation light source, resulting in a detectable fluorescence emission (Wang, C., et al. Science 291:2390-2 (2001)). In some embodiments, they have a substantially uniform size of less than 200 Angstroms or have a substantially uniform size in the range of sizes of between from about 1 nm to about 5 nm, or less than 1 nm. Methods for making semiconductor nanocrystals are known in the art. One nonlimiting method of making semiconductor nanocrystals is by a continuous flow process (e.g., see U.S. Pat. No. 6,179,912). In some embodiments, quantum dots are comprised of a Group II-VI semiconductor material (e.g., ZnS or CdSe), or a Group III-V semiconductor material (e.g., GaAs). However for some embodiments, a desirable feature of quantum dots when used for nonisotopic detection applications is that the quantum dots are water-soluble. The following provide descriptions related to nanocrystals, quantum dots, semiconductor nanocrystal, and the like: U.S. Pat. Nos. 6,838,243; 6,955,855 and 7,060,252.

Semiconductor nanocrystals can be made from essentially any material and by any technique that produces semiconductor nanocrystals having emission characteristics useful in the methods, articles, assays and compositions taught herein. Semiconductor nanocrystals have absorption and emission spectra that typically depend on their size, size distribution and composition. Suitable methods of production are disclosed, for example, in U.S. Pat. Nos. 6,048,616; 5,990,479; 5,690,807; 5,505,928; or 5,262,357; PCT Publication No. WO 99/26299; Murray et al., J. Am. Chem. Soc. 115:8706-8715; and Guzelian et al., J. Phys. Chem. 100:7212-7219 (1996).

Semiconductor nanocrystals typically have a uniform nanometer size. A semiconductor nanocrystal is capable of emitting electromagnetic radiation upon excitation (e.g., the semiconductor nanocrystal is luminescent). A semiconductor nanocrystal typically includes a “core” of one or more first semiconductor materials, which may be surrounded by a “shell” of a second semiconductor material. A semiconductor nanocrystal core surrounded by a semiconductor shell is referred to as a “core/shell” semiconductor nanocrystal. In some embodiments, a surrounding “shell” material will have a bandgap energy that is larger than the bandgap energy of a core material and may be chosen to have an atomic spacing close to that of the “core” substrate.

The core and/or the shell can be a semiconductor material including, but not limited to, those of the group II-VI (ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, and the like) and III-V (GaN, GaP, GaAs, GaSb, InN, InP, lnAs, InSb, and the like) and IV (Ge, Si, and the like) materials, Pb, PbS, PbSe, and an alloy or a mixture thereof and alloys of any semiconducting material(s). In some embodiments, a core and/or the shell can be a semiconductor material including, but not limited to, AlS, AlP, and AlSb. In some embodiments of the invention, nanocrystals have a core comprising compounds selected from the group consisting of CdSe, CdS, CdTe (collectively referred to as “CdX”). See, e.g., Norris et al., Physical Review B. 53:16338-16346 (1996); Nirmal et al., Nature 383:802-804 (1996). In some embodiments of the invention, a shell which is typically used to passivate CdX core nanocrystals is comprised of YZ wherein Y is Cd or Zn, and Z is S, or Se, or even Te. Semiconductor nanocrystals having a CdX core and a YZ shell are described in, e.g., Danek et al., Chem. Mater. 8:173-179 (1996); Dabbousi et al., J. Phys. Chem. B 101:9463 (1997); Rodriguez-Viejo et al., Appl. Phys. Lett. 70:2132-2134 (1997); and Peng et al., J. Am. Chem. Soc. 119:7019-7029 (1997).

The composition, size and size distribution of a semiconductor nanocrystal can affect its absorption and/or emission spectra. Exemplary semiconductor nanocrystals that emit energy in the visible range include, but are not limited to, CdS, CdSe, CdTe, ZnSe, ZnTe, GaP, and GaAs. Exemplary semiconductor nanocrystals that emit energy in the near IR range include, but are not limited to, InP, lnAs, InSb, PbS, and PbSe. Exemplary semiconductor nanocrystals that emit energy in the blue to near-ultraviolet include, but are not limited to, ZnS and GaN. The size of semiconductor nanocrystals in a given population can be determined, for example, by the synthetic scheme used and/or through use of separation schemes, including for example size-selective precipitation and/or centrifugation. The separation schemes can be employed at an intermediate step in the synthetic scheme or after synthesis has been completed. For a given composition, larger semiconductor nanocrystals typically absorb and emit light at longer wavelengths than smaller semiconductor nanocrystals. Semiconductor nanocrystals typically absorb strongly in the visible and UV and can be excited efficiently at wavelengths shorter than their emission peak. This characteristic allows the use in a mixed population of semiconductor nanocrystals of a single excitation source to excite all the semiconductor nanocrystals if the source has a shorter wavelength than the shortest semiconductor nanocrystal emission wavelength within the mixture; it also confers the ability to selectively excite subpopulation(s) of semiconductor nanocrystals within the mixture by judicious choice of excitation wavelength.

In some embodiments, a surface of a semiconductor nanocrystal is modified to enhance emission efficiency by adding an overcoating layer to form a “shell” around the “core” semiconductor nanocrystal, because defects in the surface of the core semiconductor nanocrystal can trap electrons or holes and degrade its electrical and optical properties. Addition of an insulating shell layer removes nonradiative relaxation pathways from an excited core, resulting in higher luminescence efficiency. In some embodiments, materials for the shell are semiconductor materials having a higher bandgap energy than the core and, in some instances, also having good conductance and valence band offset. In some embodiments, it is advantageous to have the conductance band of the shell of a higher energy and the valence band of a lower energy than those of the core. In some embodiments, nanocrystal cores that emit energy in the visible (e.g., CdS, CdSe, CdTe, ZnSe, ZnTe, GaP, GaAs) or near IR (e.g., InP, InAs, InSb, PbS, PbSe), a material that has a bandgap energy in the ultraviolet may be used for the shell, for example ZnS, GaN, and magnesium chalcogenides, e.g., MgS, MgSe, and MgTe. In some embodiments, a semiconductor nanocrystal core that emits in the near infra-red, contains materials having a bandgap energy in the visible, such as CdS or CdSe, or the ultraviolet may be used. Preparation of core-shell semiconductor nanocrystals is described in, e.g., Dabbousi et al. J. Phys. Chem. B 101:9463 (1997); Kuno et al., J. Phys. Chem. 106:9869 (1997); Hines et al., J. Phys. Chem. 100:468; PCT Publication No. WO 99/26299; and U.S. Pat. No. 6,207,229. Semiconductor nanocrystals can be made further luminescent through overcoating procedures, e.g., as described in Danek et al. Chem. Mat. 8(1):173-180 (1996), and Peng et al. J. Am. Chem. Soc. 119:7019-7029 (1997).

In some embodiments, semiconductor nanocrystals are prepared in coordinating solvent, such as trioctylphosphine oxide (TOPO) and trioctylphosphine (TOP), resulting in the formation of an organic layer (e.g., a passivating organic layer) on the surface of semiconductor nanocrystals with and without a shell. Such passivated semiconductor nanocrystals can typically be readily solubilized in organic solvents, for example toluene, chloroform or hexane. In some embodiments, molecules in a passivating layer can be displaced and/or modified to provide an outermost coating that adapts the semiconductor nanocrystals for use in other solvent systems, for example aqueous systems.

In some embodiments, an outermost layer of an inorganic material such as silica can be added around a shell to improve the aqueous dispersibility of the semiconductor nanocrystals, and the surface of the silica can optionally be derivatized (Bruchez et al., Science 281:2013 (1998)).

In some embodiments, a displacement reaction may also be employed to modify a semiconductor nanocrystal to improve the solubility in a particular solvent (e.g., organic or aqueous). For example, if it is desired to associate the semiconductor nanocrystals with a particular solvent or liquid, such as pyridine, the surface can be specifically modified with pyridine or pyridine-like moieties which are soluble or miscible with pyridine to ensure solvation. Water-dispersible semiconductor nanocrystals can be prepared, for example, as described in PCT Publication No. WO 00/17655.

A semiconductor nanocrystal can be optionally surrounded by a “coat” of an organic capping agent. The organic capping agent may be any number of materials, but typically has an affinity for the semiconductor nanocrystal surface. In general, the capping agent can be, but is not limited to, an isolated organic molecule, a polymer (or a monomer for a polymerization reaction), an inorganic complex, or an extended crystalline structure. A coat can be used to convey solubility, e.g., an ability to disperse a coated semiconductor nanocrystal homogeneously into a chosen solvent, functionality, binding properties, and/or the like. In addition, a coat can be used to tailor optical properties of a semiconductor nanocrystal. Thus, the terms “semiconductor nanocrystal” or “quantum dot” as used herein include a coated semiconductor nanocrystal core, as well as a core/shell semiconductor nanocrystal.

The surface layer of a semiconductor nanocrystal may be modified by displacement to render the semiconductor nanocrystal reactive for a particular reaction, e.g., a coupling reaction. For example, displacement of TOPO moieties with a group containing a carboxylic acid moiety enables the reaction of modified semiconductor nanocrystals with amine containing moieties to provide an amide linkage. For examples of these (linking) reactions, see, e.g., U.S. Pat. No. 5,990,479; Bruchez et al., Science 281:2013-2016 (1998); Chan et al., Science 281:2016-2018 (1998); Bruchez, “Luminescent SCNCs: Intermittent Behavior and use as Fluorescent Biological Probes” (1998) Doctoral dissertation, University of California, Berkeley; and Mikulec “SCNC Colloids: Manganese Doped Cadmium Selenide, (Core) Shell Composites for Biological Labeling, and Highly Fluorescent Cadmium Telluride” (1999) Doctoral dissertation, Massachusetts Institute of Technology. In some embodiments, a semiconductor nanocrystal may be conjugated to moieties directly or indirectly through a linker.

Examples of suitable spacers or linkers include, but are not limited to, polyethyleneglycols, dicarboxylic acids, polyamines and alkylenes. In some embodiments, spacers or linkers are optionally substituted with functional groups, for example hydrophilic groups such as amines, carboxylic acids and alcohols or lower alkoxy group such as methoxy and ethoxy groups. In some embodiments, a spacer will have an active site on or near a distal end. In some embodiments, active sites are optionally protected initially by protecting groups. Protecting groups which are useful include, but are not limited to, FMOC, BOC, t-butyl esters, t-butyl ethers, and the like. Various exemplary protecting groups are described in, for example, Atherton et al., Solid Phase Peptide Synthesis, IRL Press (1989).

In some embodiments of the invention, a diameter of a nanocrystal or the average diameter of a population of nanocrystals is between from about 0.1 nm to about 100 nm, about 1 nm to about 100 nm, about 1 nm to about 1000 nm, about 0.1 nm to about 1 nm, about 1 nm to about 50 nm, from about 2 nm to about 50 nm, from about 5 nm to about 50 nm, from about 10 nm to about 50 nm, from about 15 nm to about 50 nm, from about 20 nm to about 50 nm, from about 25 nm to about 50 nm, from about 30 nm to about 50 nm, from about 35 nm to about 50 nm, from about 40 nm to about 50 nm, from about 45 nm to about 50 nm, from about 1 nm to about 45 nm, from about 1 nm to about 40 nm, from about 1 nm to about 35 nm, from about 1 nm to about 30 nm, from about 1 nm to about 25 nm, from about 1 nm to about 20 nm, from about 1 nm to about 15 nm, from about 1 nm to about 10 nm, from about 1 nm to about 3 nm, from about 1 nm to about 5 nm, from about 5 nm to about 10 nm, from about 10 nm to about 15 nm, from about 15 nm to about 20 nm, from about 20 nm to about 25 nm, from about 25 nm to about 30 nm, from about 30 nm to about 35 nm, from about 35 nm to about 40 nm, from about 40 nm to about 45 nm, from about 45 nm to about 50 nm, from about 50 nm to about 55 nm, from about 55 nm to about 60 nm, from about 60 nm to about 65 nm, from about 65 nm to about 70 nm, from about 70 nm to about 75 nm, from about 75 nm to about 80 nm, from about 80 nm to about 85 nm, from about 85 nm to about 90 nm, from about 90 nm to about 95 nm, from about 95 nm to about 100 nm, from about 100 nm to about 200 nm, from about 200 nm to about 300 nm, from about 300 nm to about 400 nm, from about 400 nm to about 500 nm, from about 500 nm to about 600 nm, from about 600 nm to about 700 nm, from about 700 nm to about 800 nm, from about 800 nm to about 900 nm, from about 900 nm to about 1000 nm, or from about 2 nm to about 20 nm. In some embodiments, a nanocrystal or the average diameter of a population of nanocrystals is, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29 or about 30 nm.

In some embodiment of the invention, a nanocrystal is a doped metal oxide (“dMO”) nanocrystal, semiconductor nanocrystal, or combinations thereof. dMO nanocrystals are nanocrystals that can be excited, e.g., with a single excitation light source, resulting in a detectable fluorescence emission. In some embodiments of the invention, dMO nanocrystals are utilized as labels, e.g., for binding molecules and/or agents. In some embodiments, dMO nanocrystals are comprised of metal oxides doped with one or more rare earth elements, wherein the dopant comprising the rare earth element is capable of being excited (e.g., with ultraviolet light) to produce a narrow spectrum of fluorescence emission. Methods for making dMO nanocrystals are known to include, but are not limited to, a sol-gel process (e.g., see U.S. Pat. No. 5,637,258), and an organometallic reaction. A desirable feature of dMO nanocrystals when used for nonisotopic detection applications is that the nanocrystals be water-soluble. “Water-soluble” is used herein to mean that the nanocrystals are sufficiently soluble or suspendable in an aqueous-based solution including, but not limited to, water, water-based solutions, and buffer solutions, that are used in a detection process or assay.

In some embodiments, the water-solubility of a semiconductor nanocrystal is enhanced by adding to a semiconductor nanocrystal a layer comprising mercaptocarboxylic acid (Chen and Nie, Science 281:2016-2018 (1998)), or silica (e.g., see Bruchez, Jr. et al., Science 281:2013-2015 (1998) and U.S. Pat. No. 5,990,479), or one or more layers of amino acids (U.S. Pat. No. 6,114,038). Depending on which layer composition is used, the treated nanocrystal may have limited stability in an aqueous solution, particularly when exposed to air (oxygen) and/or light. More particularly, oxygen and light can, in some cases, cause molecules comprising a layer to become oxidized, thereby forming disulfides which, in some instances, can destabilize the attachment of the layer molecules to the semiconductor nanocrystals. Thus, oxidation may cause the layer molecules to become detached from the surface of the quantum dots, there-by exposing the surface of the quantum dots which may result in “destabilized quantum dots”. Destabilized quantum dots, in some cases, may form aggregates when they interact together, and the formation of such aggregates may eventually lead to irreversible flocculation of the quantum dots. Depending on the layer composition, it can cause non-specific binding, particularly to one or more molecules in a sample other than the target molecule (e.g., agent).

Some embodiments of the present invention, utilize fluorescent nanocrystals which are encapsulated by a vesicle or capsid comprising a liposome (e.g., see U.S. Pat. No. 7,060,252). In some embodiments of the present invention, fluorescent nanocrystals are encapsulated by or trapped within a vesicle or capsid comprising a liposome. In some embodiments, the surface of a liposome is functionalized with surface groups comprising a reactive functionality, e.g., that may be used to form a bond with one or more molecules of an affinity molecule which has a reactive functionality which is capable of forming a bond with surface groups of the liposome. In some embodiments, a functionalized, encapsulated fluorescent nanocrystal comprises one or more fluorescent nanocrystals encapsulated by or trapped within a liposome which is functionalized by the addition of one or more affinity molecules. In some embodiments, the present invention utilizes a functionalized, encapsulated fluorescent nanocrystal which comprises one or more fluorescent nanocrystals encapsulated by or trapped within a liposome. In some embodiments, the liposome portion may be disrupted to release fluorescent nanocrystals in a method of “quenching” the fluorescence in a reaction (e.g., see U.S. Pat. No. 7,060,252).

In some embodiments of the invention, nanocrystals comprising nanocrystals coated with an imidazole-containing compound are utilized, e.g., as labels for a detection assay. In some embodiments, a nanocrystal(s) comprises a nanocrystal coated with an imidazole containing compound and is cross-linked with a phosphine cross-linking compound.

In some embodiments of the invention, nanocrystals formed into three dimensional dendrimers are utilized. These dendrimers can function to generate and significantly amplify a detectable signal, see, e.g., U.S. Pat. No. 6,261,779.

In some embodiments, nanocrystals can be utilized to label nucleobases, providing fluorescence-labeled nucleobases, e.g., for nucleic acid strand synthesis or nucleic acid sequence detection (see, e.g., U.S. Pat. No. 6,221,602). In some embodiments, nanocrystals can be utilized to label proteins, polypeptides or peptides.

By exposing the labels comprising semiconductor nanocrystal, e.g., as prepared and described herein, to light of an excitation source, a semiconductor nanocrystals can be excited to emit light. In some embodiments, an excitation source is of an energy capable of exciting at least one population of semiconductor nanocrystals used in an experiment or assay to emit light and, in some cases, chosen to be of higher energy than the shortest emission wavelength of the semiconductor nanocrystals used. Further, the excitation source can be chosen such that it excites a minimum number of semiconductor nanocrystals in a sample(s) to produce detectable light. In some embodiments, an excitation source will excite a sufficient number of different populations of semiconductor nanocrystals to allow unique identification of the different populations of semiconductor nanocrystals used in the experiment. For example, using two different populations of binding molecules labeled with different ratios of red to blue semiconductor nanocrystals, it would not necessarily be sufficient to only excite the red emitting semiconductor nanocrystals, e.g., by using green or yellow light, of the assay. Typically, one would use a light source comprising at least one wavelength that is capable of exciting the blue emitting and the red emitting semiconductor nanocrystals simultaneously, e.g., violet or ultraviolet. In some embodiments, there may be one or more light sources used to excite different populations of semiconductor nanocrystals simultaneously, or sequentially, but a given light source may only excite subpopulations of semiconductor nanocrystals that emit at lower energy than the light source, due to the absorbance spectra of the semiconductor nanocrystals. In addition, one must consider that if a lamp source is used, degradation of the lamp may result in changes in the excitation source.

### Assay Chambers and Reactive Surface

The term “assay chamber” refers to a chamber, substrate, surface, or device where compounds of an assay of the invention interact, e.g., where binding of an agent occurs. An assay chamber is not limited to a chamber per se, but is a container or surface in or on which an assay or binding takes place. For example, if an assay is a sandwich assay, then the “capture” of the agent by a first binding molecule and binding of the captured agent by a second binding molecule occurs in or on an “assay chamber”.

In some embodiments, an assay chamber comprises a reactive surface. The term “reactive surface” refers to a surface where the binding and/or detection of the agent occurs. Using a sandwich assay format as an example, the capture binding molecule (e.g., an antibody capable of binding the agent) is typically bound to the reactive surface. A reactive surface can be essentially of any material which is compatible for the described assay(s). For example, if the assay is a sandwich assay then the surface comprises material to which a capture binding molecule(s) can be bound or associated. A reactive surface or substrate may take essentially any form including, but not limited to, a plate, slide, cover slip, bead, pellet, disk, particle, strand, precipitate, membrane, porous gel, sheet, tube, sphere, container, capillary, pad, slice, film, chip, multiwell plate or dish, optical fiber, etc.

The present invention provides various types and designs of assay chambers as described herein. These assay chambers can be used with detection apparatuses as described herein or with other devices for performing an assay. In some embodiments, an assay chamber is not used with a device per se, e.g., all assay reagents are introduced and removed manually. Assay chambers of the invention can be utilized for various assay formats, e.g., as described herein.

Some embodiments of the invention provide an assay chamber that is capable of analyzing multiple agents. Some of the embodiments of the invention are designed so as to test for a different agent(s) or a panel of different agents by using different assay chambers (e.g., channels of a flow cell) and corresponding assay reagents. This provides the advantage of a detection apparatus that can be utilized for the detection and/or analysis of essentially any agent by only changing the assay chamber and assay reagents. Some embodiments of the invention provide “disposable” assay chambers. In some embodiments, an assay chambers may be optionally archived following performance of an assay. In some embodiments, an assay chamber is a flow cell. In some embodiments, an assay chamber is a volumetrically distinct container, e.g., of varying size.

In some embodiments the reactive surface is glass, e.g., a 3-aminopropyltriethoxysilane (also known as APS, AES, APES or SILANE) treated glass. In some embodiments, the surface area is on a standard glass microscope slide or cover slip, either treated or untreated. In some embodiments, the surface is at least part of a Corning® GAPS slide (Corning, Acton, Mass.), a Corning® UltraGAPS slide, a Corning® GAPS II slide, a Corning® CMT-GAPS™ slide, or a polylysine coated glass or slide. In some embodiments, slides are produced by a commercial supplier, for example by Erie Scientific Company, Portsmouth, N.H. In some embodiments of the invention, the reactive surface or substrate comprises a component selected from the group consisting of a polymerized Langmuir Blodgett film, a functionalized glass, Si, Ge, GaAs, GaP, SiO₂, SiN₄, a modified silicon, or anyone of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonate, or combinations thereof

Surfaces on a substrate or reactive surface can be composed of the same material as the substrate or reactive surface or can be made from a different material, and can be coupled to the substrate by chemical or physical means. Such coupled surfaces may be composed of any of a wide variety of materials, for example, polymers, plastics, resins, polysaccharides, silica or silica-based materials, carbon, metals, inorganic glasses, membranes, or any of the above-listed substrate or reactive surface materials. In one embodiment, a surface will be optically transparent and/or will have surface Si-OH functionalities, such as those found on silica surfaces.

A substrate or reactive surface may be chosen to provide appropriate optical characteristics for the detection methods used. A substrate and/or surface can be transparent to allow the exposure by light applied from one or multiple directions. A substrate and/or surface may be provided with reflective “mirror” structures to increase the recovery of light emitted, e.g., by a semiconductor nanocrystal or other label. A substrate and/or its surface may also be coated to decrease the amount of spurious incident light. The optical density of a substrate or surface may be designed according to an assay method.

A substrate and/or its surface may be of a material which is resistant to, or is treated to resist, the conditions to which it is to be exposed in use, and can be optionally treated to remove any resistant material after exposure to such conditions.

Targets or capture binding molecules can be fabricated on or attached to a substrate or reactive surface by any suitable method, for example the methods described in U.S. Pat. No. 5,143,854; PCT Publication Nos. WO 92/10092 and WO 90/15070; and Fodor et al., Science 251:767-777 (1991). Techniques for the synthesis of arrays using mechanical synthesis strategies are described in, e.g., PCT Publication No. WO 93/09668 and U.S. Pat. No. 5,384,261 which can be utilized with the present invention. Guidance for fabrication, sample labeling and conditions for hybridization are described, for example, in Bittner, et al. Nature 406:536-540 (2000); Khan, et al. Electrophoresis 20:223-9 (1999); Duggan, Science 283:83-87 (1999); and DeRisi et al., Nature Genet 14:457-60 (1996). Additional flow channel or spotting methods applicable to attachment of targets to a substrate are described in U.S. Pat. Nos. 5,384,261 and 5,677,195. In some embodiments, reagents are delivered to a substrate or reactive surface by either (1) flowing within a channel or (2) “spotting”.

A protective coating, such as a hydrophilic or hydrophobic coating (depending upon the nature of the solvent), can be used over portions of a substrate or reactive surface to be protected, optionally in combination with materials that facilitate wetting by a reactant solution in other regions. In this manner, flowing solutions are further prevented from passing outside of their designated flow paths. In some embodiments, dispensers include a micropipette, optionally robotically controlled; an ink jet printer; a series of tubes; a manifold; an array of pipettes; or the like so that various reagents can be delivered to the reaction or binding sites sequentially or simultaneously.

Considerations for preparing surfaces include the following. Any contamination of this sort may cause nonspecific light scattering. In some embodiments, background is minimized by making sure that buffers do not dry on the slides; use clean, filtered, compressed air or nitrogen to dry slides; do not use powdered gloves; handle slides with forceps if possible, especially during the final wash step. In some embodiments, chemical blocking is not performed with succinic anhydride, e.g., because it can lead to high levels of non-specific background. In some embodiments, surfaces are not washed with SDS-containing buffers after printing and prior to processing of the arrays. In the case of LS technology, it can be sensitive to contamination by dust particles or residue from dried droplets of buffers on either side of the glass slide. Also, further considerations and a troubleshooting guide are provided in “GeniconRLS™ One-Color and Two-Color Microarray Toolkits™” Instruction Manual, Version B, Jan. 18, 2005 from Invitrogen (Carlsbad, Calif.).

Therefore, the present invention provides methods of making assay chambers and or reactive surfaces. Some embodiments provide methods of making assay chambers that comprise at least one binding molecule. Also provided are methods for attaching or binding a binding molecule to an assay chamber or reactive surface.

Some assay chambers of the invention comprise a waveguide. A “waveguide” refers to a two dimensional TIR element such that light is essentially totally internally reflected at multiple points, thereby creating an evanescent wave, e.g., that is substantially uniform across all or nearly the entire surface. In some embodiments, a two dimensional waveguide may be planar in configuration. In one embodiment of the present invention, a TIR element is essentially a two dimensional waveguide.

FIGS. 11A-C illustrate an exemplary embodiment (e.g., a flow cell), wherein a waveguide device or sample chamber 30 comprises a planar waveguide element 32, a parallel planar plate 34, adhesive means and flow gasket 48, a sample port 50, circulation ports 52 and optionally identification means 54 (e.g., a bar code). The waveguide element thus has parallel surfaces 36 and 38 as well as a light-receiving edge. Similarly, the plate 34 has parallel surfaces 42 and 44. The waveguide element 32 and the plate 34 are held together in spaced parallel fashion, such that the element surfaces 38 and the plate surface 42 define a channel 46. The element and plate may be held together by any convenient means, including adhesive means on a flow gasket 48 (shown as hatched areas) consisting of double stick or two-sided tape disposed along the edges of the element and plate. In some embodiments, a channel(s) (e.g., 46) is of a size so as to enable capillary transfer of a fluid sample there through. In some embodiments, the height will typically be less than about 1 mm or less than about 0.1 mm.

In some embodiments, an assay chamber includes at least one of the following: a tape or gasket thickness to create the channel depth; using a black tape or a black gasket to reduce background noise; or masking of the slide with black material (e.g., epoxy) creating windows for the reaction sites significantly reducing background.

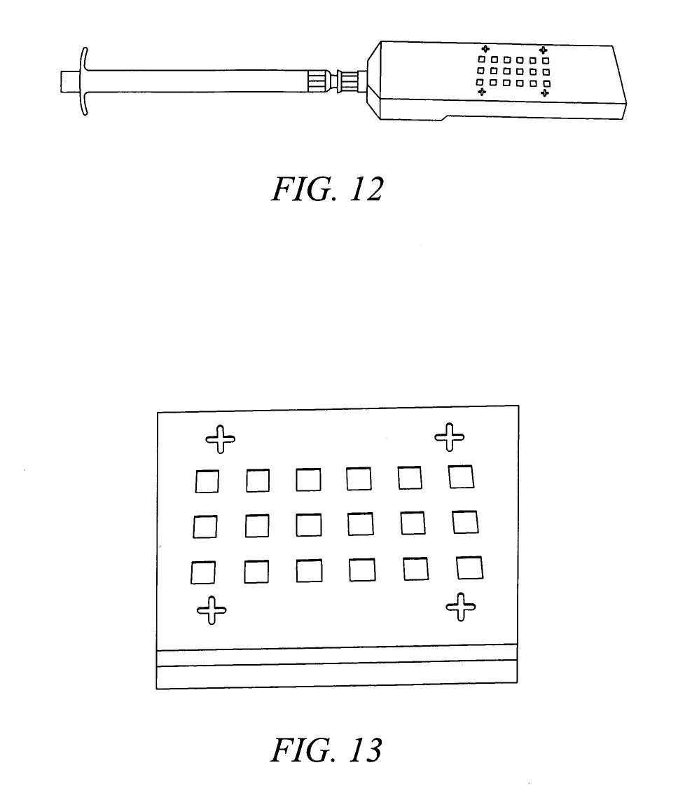

In some embodiments, an assay chamber or a flow cell will comprise a substrate with entrance and exit ports to deliver reagents through the channels. In some embodiments, a separate sample introduction port is included to allow direct injection into a flow cell. In some embodiments, a sample introduction interface is present on an assay chamber (e.g., a flow cell) wherein the sample directly enters into the flow cell, e.g., with a standard syringe into a sample port(s) (e.g., see FIG. 12). In some embodiments, the syringe is left attached to the assay chamber throughout the performance of the assay and in some instances remains on the assay chamber when the assay chamber is disposed of. This allows for a closed system during the performance of the assay.

In some embodiments, an assay chamber or flow cell is comprised of three major components a waveguide element (e.g., a glass slide), a gasket and a base (superstructure). In some embodiments, a glass slide 32 is masked and produced with AES coating, e.g., by Erie Scientific, Portsmouth, N.H. In some embodiments, a flow gasket 48 is cut to specifications. In some embodiments, a flow gasket 48 is cut via a rotary die-cutting process. Cutting a gasket to desired specifications can be performed by a commercial entity, e.g., by Brady Medical Converting. In some embodiments, a flow gasket 48 is a 3M Double coated tape #9690B with black PET carrier. In some embodiments, a base 34, sometime referred to as a superstructure, is machined to specifications, e.g., by New London Precision Instruments, Ijamsville, Md. In some embodiments, a base is injection molded. In some embodiments, a base 34 is plastic or metal. In some embodiments, a base 34 is machined or made from acrylic. In some embodiments, a base 34 is of a black color. In some embodiments, a gasket is produced with adhesive coating on both sides, and serves to hold the cell components together, as well as to define the flow channels. In some embodiments, a luer connector is threaded to the end of the sample channel for sample injection.

In some embodiments, an assay chamber or flow cell is double sided and comprises two waveguide elements (e.g., glass slides), two gaskets and a base (superstructure). Some of these embodiments are similar to the embodiment shown in FIG. 11 except a superstructure is sandwiched between two waveguide elements. Some of these types of assay chambers can be read using two cameras, e.g., one directed at each camera. Alternatively, one camera can be used wherein the camera can move (manually or automated) to record each or the two waveguides. In some embodiments, the camera does not move, but the waveguide is capable of moving so that each waveguide can be imaged. Assay chambers with one or two waveguide elements are only meant as examples. The invention provides assay chambers with essentially any number of waveguide elements. The number is limited only by the size of the assay chamber. For example, a superstructure could be 3, 4, 5, 6, 7, 8, 9, 10, 11, 12, 13, 14, 15, 16, 17, 18, 19, 20, or more sided with a waveguide element on each side. For example, in some embodiments, an assay chamber with 3 waveguide elements may comprise a superstructure with a cylindrical triangle; four could be a cylindrical rectangle or square, four could be a cylindrical pentagon; etc.

In some embodiments, a waveguide element 32 comprises a binding molecule, e.g., a capture binding molecule such as an antibody. In some embodiments, a waveguide element 32 comprises a binding molecule (e.g., an antibody) localized in distinct regions or sites or “spots” 54, e.g., positioned over/in a channel 46. In some embodiments, a waveguide element 32 comprises multiple channels. In some embodiments, a waveguide element 32 comprises at least one channel for a negative control, at least one channel for a positive control, and at least one channel for a sample.

In some embodiments, a waveguide element 32 is made of an optically transparent material such as glass, quartz, plastics such as polycarbonate, acrylic, or polystyrene. In some embodiments, a waveguide element comprises a material, typically on the “top” edge 36, that reduces or “masks” the capability of light to pass though, e.g., see FIG. 13. Typically, this material is not present directly “over” the test region(s), situs or sites where an agent is detected, e.g., creating a window “over” the test situs. In some embodiments, this material will be of a dark color such as black to decrease the amount of light “escaping” the waveguide element at regions other than the test regions or sites comprising binding molecules. This coating may also result in symbols, letters, words, etc. being displayed on the surface, e.g., a plus sign(s) (FIG. 13). These symbols, letters, words may be of any color and in some embodiments they are white. In some embodiments, words are displayed on the surface to identify assays or agents that are detectable using the flow cell or assay chamber. In some embodiments, each window is marked so as to identify the contents of at least one or each region or situs, e.g., marked as positive control, negative control, test region, or a specific agent(s) being detected in the situs or region.

A waveguide element may be comprised of a plastic or a glass, for example, a standard glass microscope slide or cover slip may be used. In some embodiments, a waveguide element may be machined or produced by injection molding. Injection molding allows for the introduction of various features during the molding process. In most embodiments, the refractive index of a waveguide is greater than the refractive index of the sample fluid or readout solution. For an aqueous readout solution, the refractive index, n, is typically about 1.33, so in some embodiments of the invention a waveguide has a refractive index of greater than 1.35, usually about 1.5 or more. In some embodiments of the invention the refractive index of a waveguide is greater than about 1.3, greater than about 1.35, greater than about 1.40, greater than about 1.45, greater than about 1.50, greater than about 1.55, greater than about 1.60 and greater than about 1.65. In some embodiments of the invention the refractive index of the waveguide is between from about 1.0 to about 6.0, from about 1.0 to about 5.0, from about 1.0 to about 4.0, from about 1.0 to about 3.0, from about 1.0 to about 2.0, from about 2.0 to about 6.0, from about 3.0 to about 6.0, from about 4.0 to about 6.0, from about 5.0 to about 6.0, from about 1.0 to about 2.0, from about 2.0 to about 3.0, from about 3.0 to about 4.0, from about 4.0 to about 5.0, from about 5.0 to about 6.0, from about 1.5 to about 2.5, from about 2.5 to about 3.5, from about 3.5 to about 4.5, from about 4.5 to about 5.5, from about 1.0 to about 1.1, from about 1.1 to about 1.2, from about 1.2 to about 1.3, from about 1.3 to about 1.4, from about 1.4 to about 1.5, from about 1.5 to about 1.6, from about 1.6 to about 1.7, from about 1.8 to about 1.9, from about 1.9 to about 2.0, from about 2.0 to about 2.1, from about 2.1 to about 2.2, etc. In some embodiments of the invention, a readout solution is less than about 1.3, less than about 1.35, less than about 1.40, less than about 1.45, less than about 1.50, less than about 1.55, less than about 1.60 and less than about 1.65. In some embodiments of the invention the refractive index of a readout solution is between from about 1.0 to about 5.0, from about 1.0 to about 4.0, from about 1.0 to about 3.0, from about 1.0 to about 2.0, from about 2.0 to about 5.0, from about 3.0 to about 5.0, from about 4.0 to about 5.0, from about 1.0 to about 2.0, from about 2.0 to about 3.0, from about 3.0 to about 4.0, from about 4.0 to about 5.0, from about 1.5 to about 2.5, from about 2.5 to about 3.5, from about 3.5 to about 4.5, from about 4.5 to about 5.5, from about 1.0 to about 1.1, from about 1.1 to about 1.2, from about 1.2 to about 1.3, from about 1.3 to about 1.4, from about 1.4 to about 1.5, from about 1.5 to about 1.6, from about 1.6 to about 1.7, from about 1.8 to about 1.9, from about 1.9 to about 2.0, from about 2.0 to about 2.1, from about 2.1 to about 2.2, from about 1.30 to about 1.35, from about 1.32 to about 1.37, from about 1.37 to about 1.42, from about 1.40 to about 1.45, from about 1.42 to about 1.47, from about 1.45 to about 1.50, from about 1.47 to about 1.52,etc.

In some embodiments, the readout solution is water or aqueous based. In some embodiments, the readout solution comprises glycerol (e.g., 50% in water). By “readout solution” is meant the solution present in an assay chamber or at reactive surface when LS is measured or detected.

In some embodiments, a base or superstructure 34 is constructed of materials such as glass, quartz, plastics such as polycarbonate, acrylic, or polystyrene. In some embodiments, a base is black. In some embodiments, the light receiving end of a waveguide element is disposed in a narrow slit of a mask, e.g., in order to minimize the effects of stray light originating from a light source. Minimization of stray light may also be improved by the use of light absorbing materials. In some embodiments, a superstructure is produce by injection molding or is machine. Injection molding allows for the introduction of various features during the molding process as opposed to adding them later or machining them in, thus saving time and possibly resources. For example, if a luer lock compatible port (e.g., sample port) is desired, it can be designed into the mold which eliminates the need to create the port and/or attach the luer lock device later. Also, a port(s) for fluidic transportation can be designed into the mold.

In some embodiments, an assay chamber comprises one or more channels. In some embodiments, channels are formed using a two sided adhesive or gasket between two planar objects. In these embodiments, two sided adhesive or gasket performs at least two functions, one being the joining of a superstructure and a waveguide element and the second being the formation of the channels. In some embodiments, a gasket is used that does not comprise an adhesive. In this embodiment, the “height” of the channel can be determined by or contributed to by the thickness of a two sided adhesive or gasket. In some embodiments, one or both of the planar objects comprise a ditch feature, which forms a channel. A ditch feature may be made by any methods including, but not limited to, machining, etching, or molding into the structure. In some embodiments, a channel is formed using a ditch feature with or without a two sided adhesive and/or gasket.

In some embodiments, an assay chamber or waveguide element comprises 1, 2, 3, 4, 5, 6, 7, 8, 9, 10, 11, 12, 13, 14, 15, 16, 17, 18, 19, 20, 21, 22, 23, 24, 25, or more channels. In some embodiments, a assay chamber or waveguide element comprises between from about 1 to about 10000, from about 1 to about 1000, from about 1 to about 100, from about 100 to about 1000, from about 500 to about 1000, from about 1 to about 10, from about 1 to about 20, from about 1 to about 30, from about 1 to about 40, from about 1 to about 50, from about 10 to about 100, from about 20 to about 100, from about 30 to about 100, from about 40 to about 100, from about 50 to about 100, from about 60 to about 100, from about 70 to about 100, from about 80 to about 100, from about 90 to about 100, from about 10 to about 20, from about 15 to about 25, from about 20 to about 30, from about 25 to about 35, from about 35 to about 45, from about 40 to about 50, from about 45 to about 55, from about 50 to about 60, from about 55 to about 65, from about 60 to about 70, from about 65 to about 75, from about 70 to about 80, from about 75 to about 85, from about 80 to about 90, from about 85 to about 95, from about 90 to about 100, from about 100 to about 200, from about 200 to about 300, from about 300 to about 400, from about 400 to about 500, from about 500 to about 600, from about 600 to about 700, from about 700 to about 800, from about 800 to about 900, from about 900 to about 1000, from about 1000 to about 3000, from about 3000 to about 7000, or from about 7000 to about 10000 channels.

Channels of an assay chamber can be of any size, radius, width and/or depth that allows for assay reagents to contact a reactive surface (e.g., containing a capture binding molecule) and allows for a detectable signal to be produced. In some embodiments, channels of an assay chamber of the invention have a width and/or depth of between from about 0.1 μm to about 1 meter, from about 1 mm to about 1 meter, from about 1 cm to about 1 meter, 10 cm to about 1 meter, 100 cm to about 1 meter, from about 0.1 μm to about 1 μm, from about 0.1 μm to about 10 μm, from about 0.1 μm to about 100 μm, from about 0.1 μm to about 1 mm, from about 0.1 μm to about 1 cm, from about 1 μm to about 10 μm meter, from about 10 μm to about 100 μm, from about 100 μm to about 1 mm, from about 1 mm to about 2 mm, from about 1.5 mm to about 2.5 mm, from about 2 mm to about 3 mm, from about 2.5 mm to about 3.5 mm, from about 3 mm to about 4 mm, from about 3.5 mm to about 4.5 mm, from about 4 mm to about 5 mm, from about 4.5 mm to about 5.5 mm, from about 5 mm to about 6 mm, from about 6.5 mm to about 7.5 mm, from about 7 mm to about 8 mm, from about 8.5 mm to about 9.5 mm, from about 9 mm to about 1 cm, from about 9.5 mm to about 1.5 cm, from about 1 cm to about 2 cm, from about 2 cm to about 3 cm, from about 1 cm to about 10 cm, from about 10 cm to about 100 cm, from about 3 cm to about 7 cm, or from about 7 cm to about 10 cm. In some embodiments, the radius, width and/or depth of a channel is about 0.1 mm, about 0.2 mm, about 0.28 mm, about 0.3 mm, about 0.4 mm, about 0.5 mm, about 0.6 mm, about 0.7 mm, about 0.8 mm, about 0.8 mm, about 0.9 mm, about 1 mm, about 1.15 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, or about 10.5 mm. In some embodiments, a cross section of a channel is circular, rectangular, a square or elliptical shape. In some embodiments, a two dimensional cross section of a channel comprises an area between from about 0.1 μm² to about 1 meter², from about 1 mm² to about 1 meter², from about 1 cm² to about 1 meter², 10 cm² to about 1 meter², 100 cm² to about 1 meter², from about 0.1 μm² to about 1 μm², from about 0.1 μm² to about 10 μm², from about 0.1 μm² to about 100 μm², from about 0.1 μm² to about 1 mm², from about 0.1 μm² to about 1 cm², from about 1 μm² to about 10 μm² meter, from about 10 μm² to about 100 μm², from about 100 μm² to about 1 mm², from about 1 mm² to about 2 mm², from about 1.5 mm² to about 2.5 mm², from about 2 mm² to about 3 mm², from about 2.5 mm² to about 3.5 mm², from about 3 mm² to about 4 mm², from about 3.5 mm² to about 4.5 mm², from about 4 mm² to about 5 mm², from about 4.5 mm² to about 5.5 mm², from about 5 mm² to about 6 mm², from about 6.5 mm² to about 7.5 mm², from about 7 mm² to about 8 mm², from about 8.5 mm² to about 9.5 mm², from about 9 mm² to about 1 cm², from about 9.5 mm² to about 1.5 cm², from about 1 cm² to about 2 cm², from about 2 cm² to about 3 cm², from about 1 cm² to about 10 cm², from about 10 cm² to about 100 cm², from about 3 cm² to about 7 cm², or from about 7 cm² to about 10 cm².

In some embodiments, an assay chamber is a flow cell that will be used, e.g., to run an assay to detect or analyze multiple agents. In some embodiments, an assay chamber will comprise multiple sites, wherein a situs comprises binding molecules that bind an agent or agents.

A “situs” (plural=“sites” herein) is a distinct or a delimited area, e.g., on a reactive surface or assay chamber. In some embodiments, situs comprises a specific binding molecule(s) for an agent. In some embodiments, the binding molecule(s) is immobilized. It is understood that non-situs portions of the surface will also exist outside of a delimited area.

An assay chamber or reactive surface may comprise one or multiple sites. In some embodiments, a reactive surface or assay chamber comprises at least two sites wherein the at least two sites bind the same agent(s) or bind different agents. In some embodiments, an assay chamber comprises a first situs that binds an agent and a control situs that acts as a control for at least one part of the assay. For example, the control situs can be a positive control or negative control. In some embodiments, multiple sites comprise the same binding molecule and are exposed to the same samples. These sites can act as replicates during data analysis. In some embodiments, an assay chamber comprises 2, 3, 4, 5, 6, 7, 8, 9, 10, or more replicates. In some embodiments, multiple sites of an assay chamber or reactive surface comprise the same binding molecule and are exposed to different samples. In some embodiments, the multiple sites can be in one channel or multiple channels. In some embodiments, an assay chamber comprises at least three sites comprising 1) a test situs to bind an agent, if present, in a sample; 2) a negative control situs that acts as a negative control; and a 3) a positive control situs that acts as a positive control. Using FIG. 11 as an example, the figure shows 18 sites and 6 sites per channel.

In some embodiments of the invention, a reactive surface comprising at least one situs is formed, e.g., on one side of a waveguide element. While some embodiments may have only a single test situs, some embodiments of the invention also utilize a plurality of such sites. Multiple test sites may contain the same or different specific binding molecules. In some embodiments, an immobilized specific binding molecule(s) is referred to herein as a “capture binding molecule”. In some embodiments, a situs is a small spot or dot. In some embodiments, the non-situs portions surround a situs. Of course many other situs sizes and configurations are possible and within the invention. A situs may also be configured as a line or bar; as a letter or numeral; as a circle, rectangle, triangle, square, or as any other graphic such as, for example, any graphic typically employed in computer icon or clip-art collections.

In some embodiments of the invention, a situs configuration is the shape of a cross, which results in a “plus” symbol in the event of a positive result. In some embodiments, only the one line (e.g., the vertical portion or portions) of the plus sign contains agent binding molecules, while the other line (e.g., the horizontal portion or portions) of the plus sign contains a label which is detectable independent of the presence of an agent. Besides the “plus/minus” verification configuration, other shapes of this variation are also possible.

In some embodiments, a negative control situs is “spotted” using the same methods and reagents as the corresponding test situs. In some embodiments, a negative situs is “spotted” using the same methods and reagents as the test situs, except the capture binding molecule is replaced with a related molecule that does not specifically bind the agent. For example, an antibody that binds the agent is replaced with an antibody (e.g., of the same isotype as the capture antibody that binds the agent(s)) that does not bind the agent(s). In some embodiments, a positive control situs comprises a binding molecule or mix of different binding molecules that do not bind the agent, but bind another agent of interest known to be in a sample, thus acting as a positive control for the assay. The binding molecules of a positive situs may bind to an agent naturally present in a sample e.g., IgG antibodies if the sample is serum or a ubiquitous plant protein if the sample is plant tissue. In some embodiments, the binding molecules of a positive situs may bind an agent that is “spiked” into a sample, therefore acting as a positive control for the assay. In some embodiments, a region comprises sites comprising at least one test situs, at least one positive control situs and at least one negative control situs. In some embodiments, a assay chamber or reactive surface does not comprise a positive control situs and/or a negative control situs. In some embodiments, a waveguide element comprises a negative control region and/or a positive control region. Using FIG. 11 as an example, there are 6 sites per channel. Typically each channel will be contacted with one sample, e.g., a test sample, a positive or negative control sample.

In some embodiments, the size of a situs is limited only by the resolution and/or magnification limits of the system. In some embodiments, a situs is contained within or has an area of about 0.1 μm², 1 μm², 1 μm², 10 μm², 80 μm², 100 μm², 1 mm², 2 mm², 3 mm², 4 mm², 5 mm², 6 mm², 7 mm², 8 mm², 9 mm², or 10 mm². In some embodiments, a situs is contained within or has an area between from about 0.01 μm² to about 10 mm², about 0.01 μm² to about 0.1 μm², about 0.01 μm² to about 1 μm², about 0.1μm² to about 1 μm², about 1 μm² to about 10 μm², about 1 μm² to about 100 μm², about 10 μm² to about 100 μm², about 50 μm² to about 100 μm², about 70 μm² to about 80 μm², about 75 μm² to about 85 μm², about 75 μm² to about 100 μm², about 10 μm² to about 1 mm², about 100 μm² to about 1 mm², about 1 mm² to about 10 mm², about 1 mm² to about 2 mm², about 1 mm² to about 3 mm², about 3 mm² to about 4 mm², about 4 mm² to about 5 mm², about 5 mm² to about 6 mm², about 6 mm² to about 7 mm², about 7 mm² to about 8 mm², about 8 mm² to about 9 mm², or about 9 mm² to about 10 mm².

In some embodiments, the area (size) of a situs need be large enough only to immobilize a sufficient amount of a binding molecule(s) to enable capture and detection of the agent, e.g., by RLS. This is dependent in part on the density of the situs. Small areas are preferred when many sites will be placed on a reactive surface, giving a high “site density”. In some embodiments utilizing visual detection, areas large enough to be detected without magnification can be used or large enough areas to be used with compatible magnification methods, for example about 1 to about 50 mm², or 1 cm² or even larger. There is essentially no upper size limit except as dictated by manufacturing costs and user convenience and any desired situs size or shape is suitable. The size of a situs may be optimized for a desire detection level.

In some embodiments, the density (quantity per unit area) of a capture binding molecule(s) on a reactive surface typically correlates positively with the sensitivity of the system to a point. Extremely high densities may provide sub-optimal performance, e.g., due to steric restrictions imposed. Optimal density for best sensitivity typically involves a trade off between maximizing the number of binding sites per unit area, and maximizing the access to such sites keeping in mind diffusion kinetics requirements and steric considerations.

Application of a capture binding molecule onto a reactive surface may be accomplished by any convenient means. For example, manual or automated use of micropipetters or microcapillary tubes may be conveniently used for spotting or spraying a population of a capture binding molecule(s) onto a reactive surface. Some embodiments of the invention use an automated process, e.g., for convenience, reproducibility or cost-savings. Automated application methods include, for example, positive displacement pumps, X-Y positioning tables, and/or ink jet spraying or printing systems and the like. In some embodiments, a capture binding molecule is spotted onto a surface using a high-throughput instrument such as the BioDot Arrayer (BioDot, Inc, Irvine, Calif.) or similar device.

When appropriate, the binding molecules may first be put into a solution to facilitate a process of depositing the samples onto the reactive surface. In some embodiments, a suitable solution will upon drying, allow the binding molecule to retain or retain a portion of its specificity and/or binding properties, and does not significantly interfere with the refractive properties of the element. In some embodiments, a crosslinking agent is included to increase the amount of binding molecule at the capture site, provided the crosslinking agent still allows the binding molecule to bind an agent.

In some embodiments, after the binding molecule has been deposited on one or more sites of the surface, the binding molecule solution is allowed to dry and thereby the binding molecule becomes immobilized on the surface. Drying may be performed at room temperature (e.g., about 25° C., ambient temperature or another suitable temperature). When desired, the evaporation/drying may be performed at elevated temperature, so long as the temperature does not significantly inhibit the ability of the binding molecule(s) to specifically interact with its corresponding binding partner or agent. For example, where the immobilized capture binding molecule is a protein, non-denaturing temperatures should be employed. Additionally, drying can occur at reduced pressure.

In some embodiments, a capture binding molecule is deposited on a reactive surface using photolithographic methods. There exists a number of commercially available heterobifunctional photoactivatable crosslinkers (PACs) that may be employed to allow for the photolithographic addressing of a binding molecule(s) (e.g., an antibody or nucleic acid) on an array or reactive surface. PACs typically have in common an aryl-azido moiety that upon excitation with UV radiation decomposes yielding a reactive nitrene. This nitrene reacts to form a covalent bond with other molecules that contain amines such as proteins. In some embodiments a glass surface can be functionalized with, for example, a silyl-amino reagent and subsequently derivatized with the PAC. Binding molecules such as antibodies can then be delivered, e.g., fluidically, to an array or surface, and spots of interest would be irradiated to afford conjugation. In some embodiments a binding molecule such as an antibody could be derivatized with a PAC and then applied to an amino-functionalized array with subsequent irradiation of specific spots. One consideration is non-specific binding (NSB) of a binding molecule (e.g. an antibody) to the surface. As a class of molecules proteins have a wide variation of hydrophobic profiles, and they bind with a range of avidities to surfaces. The nature of a surface may have an impact on the extent of binding by the protein. In some embodiments, a surface is passivated with a hydrophilic polymer such as poly(ethylene glycol) or a protein such as bovine serum albumin or casein is used. In some embodiments, passivated surfaces can be functionalized in order to utilize the PAC-mediated conjugation of the binding molecules such as antibodies.

In some embodiments, a capture binding molecule (e.g. an antibody or antibody fragment) is deposited on a surface at one or more sites using a BioDot arrayer (e.g., Model #: AD3200, BioDot, Inc. Irvine, Calif.). In some embodiments of the invention, a capture binding molecule is deposited in distinguishable sites to form assay replicates. In some embodiments, distinguishable sites of replicates are in close proximity, e.g., not separated by any other sites that, for example, contain another capture binding molecule(s). In some embodiments, distinguishable sites of replicates are not in close proximity, e.g., are separated by at least one other situs that, for example, contain another capture binding molecule(s). The number of distinguishable sites can be 2, 3, 4, 5, 6, 7, 8, 9, 10, or more. In some embodiments, the number of distinguishable sites is between from about 2 to about 1000, about 2 to about 100, about 2 to about 10, about 2 to about 8, about 2 to about 6, about 2 to about 4, about 3 to about 5, about 5 to about 10, about 10 to about 50, about 50 to about 100, about 100 to about 500, or about 500 to about 1000. In some embodiments, an assay chamber comprises 4 situs, wherein 3 are test situs and 1 is a control situs (e.g., negative control situs). Some embodiments of the invention comprise multiple sets of sites, e.g., wherein a set comprises 4 situs, wherein 3 are test situs and 1 is a control situs.

In addition to immobilization of capture binding molecules to a surface, a surface may be treated so as to block non-specific interactions, e.g., between the reactive surface and an agent in a sample which is to be tested. In the case of a protein binding molecule (e.g., an antigen, antibody or PNA) on the surface, the blocking material is typically applied after immobilization of a capture binding molecule. Suitable protein blocking materials are casein, zein, bovine serum albumin (BSA), 0.5% sodiumdodecyl sulfate (SDS) and 1× to 5× Denhardt's solution (1× Denhardt's is (0.02% Ficoll, 0.02% polyvinylpyrrolidone and 0.2 mg/ml BSA). Other blockers can be detergents and long-chain water soluble polymers. In some embodiments, a blocking material is 1% w/v Casein Hammersten Grade in PBS, Kathon as preservative, pH 7.4. The blocking material may be conveniently applied to the surface (e.g., a reactive surface) as an aqueous or buffered aqueous solution. Typically, but not necessarily, a blocking solution is applied to the surface at any time after a first capture binding molecule(s) is immobilized. For example, in the case of a nucleic acid binding molecule, the blocking material may be applied before or after immobilization of the binding molecule.

In some embodiments, the first specific binding member may be specific for the agent through the intermediary of additional cognate pairs or binding proteins. For example, a binding molecule may be biotinylated and attached to a reactive surface via a biotin-avidin cognate binding pair, e.g., see European Patent Publication No. EP 0139489. In some embodiments, the binding molecule may be attached to a reactive surface through a mediator probe, e.g., see U.S. Pat. No. 4,751,177. When using intermediary cognate binding molecules in combination with light scatter techniques, typically one must keep in mind that the total distance from the interface (at the reactive surface) to the light scattering label should not greatly exceed the penetration depth.

In some embodiments, a reactive surface is formed on the surface 38 of a waveguide element 32 which faces into a channel 46, see FIG. 11. This can facilitate the contacting of assay reagents with a situs or site on the reactive surface, e.g., by permitting flow (e.g., capillary flow) across the reactive surface. In some embodiments, flow can be enhanced by the use of an absorbent or bibulous material such as paper at one end of the channel. In some embodiments, flow is produced using a pump. In some embodiments, an assay chamber in connection with a detection apparatus is capable of continuous flow and/or a flow loop, e.g., through a channel.

In some embodiments, an assay chamber comprises at least 3 lanes with 6 sites per lane. In some embodiments, this includes one lane each for sample, positive control and negative control. One skilled in the art will recognize that the number of detection sites and/or channels could be increased or decreased to accommodate a specific use or for a particular type of analysis. In some embodiments, an assay chamber of the invention is used in the field for on site detection of bio-threat agents.

In some embodiments, an assay chamber consists of or comprises a glass surface or component (e.g., a microscope type slide) spotted with a capture binding molecule(s) of interest such as antibodies or protein ligands. In some embodiments, the slide may also act as a waveguide for illumination. Some embodiments include a layer of double sided black tape or a black gasket, e.g., which is laser cut, die cut, or water jet cut with the appropriate channels, with the tape thickness determining the desired depth of a channel(s). In some embodiments, a flow cell or assay chamber comprises 3 components: a base (e.g., 34 in FIG. 11), a double sided tape or gasket (e.g., 48 in FIG. 11), and a reactive surface (e.g., 38 in FIG. 11) on e.g., 32 in FIG. 11. In some embodiments, a base contains entry and exit ports for fluid introduction and for circulating a sample over the targets (e.g., 50 in FIG. 11). In some embodiments, double-sided tape or a gasket of appropriate thickness can be cut with a design for flow to a reaction/detection zone and a reactive surface, e.g. on a slide that has been patterned with a black mask (e.g., see FIG. 13) and/or spotted with a binding molecule (e.g., capture; e.g., see FIG. 11B). In some embodiments, the tape or gasket is cut with a laser cut, a water jet cut, and/or die cut. In some embodiments, the base is laser cut, machined and/or molded. Once a design has been determined the parts can be made and assembled. Some aspects of the invention comprise aligning slots in the tape with holes in the base and sandwiching this with the reactive surface (e.g., glass), which creates a flow path, e.g., that produces laminar flow. This along with spotting equipment for spotting binding molecules such as antibodies allows for an easily configurable format for any microfluidic flow application. In some embodiments, a detection apparatus will circulate a sample(s) and/or reagent through a sample chamber such as a flow cell. In some embodiments, a sample will be introduced with a syringe (e.g., through a check valve) into a flow cell or sample chamber. In some embodiments, pumps and valves will circulate a sample over the target zone then direct it to waste. After an appropriate time the assay chamber or slide is imaged and the results are determined.

In some embodiments, channels are cut in a substance(s) (e.g., a plastic sheets) then they are laminated together, e.g., with tape and/or another adhesive means. In some embodiments, a flow cell of the invention comprises channels that are not formed via cutting of a substance or plastic sheet.

In some embodiments, a substance such as “tape”, a gasket or equivalents is utilized to, for example to determine the channel depth. In some embodiments, a substance such as “tape”, a gasket or an equivalent that is black, dark colored, non-translucent, opaque and/or minimally translucent can be utilized, e.g., to reduce background noise.

In some embodiments, a masking substance is utilized. A masking substance or element, inter alia, can reduce background signal such as background light scattering. In some embodiments, a masking substance or element reduces or blocks reflection and/or refraction. In some embodiments, mask may serve to reduce background light, and to isolate and simplify detection of and differentiation between positive and negative samples. In some embodiments, a masking of the slide of a flow cell or waveguide element with a black, dark colored, opaque, non-translucent and/or minimally translucent substance is utilized, for example creating windows for reaction sites, e.g., see FIG. 13. This may lead to significantly reducing background. In some embodiments, masking can be done with a rough grain to avoid light reflections. In some embodiments, the substance can be on the same side of a waveguide element as the side with at least one situs. In some embodiments, the substance can be on the opposite side of a waveguide element as the side with at least one situs. In some embodiments, the substance can be on both the same and opposite side of a waveguide element as the side with at least one situs. In some embodiments, the masking substance or element is not present over a situs or in line with a situs and a detection device. In some embodiments, a mask substance or element is used to block or diminish light or a signal from a non-situs area. A mask element or substance can be of essentially any material which reduces, diminishes or blocks light or a detectable signal. In some embodiments, a mask element or substance comprises Teflon or epoxy. In some embodiments, a mask element or substance comprises a black color. In some embodiments, a mask element is black except for marking in or on the mask, e.g., for labeling or aligning an assay chamber or waveguide element.

For clarity, a two-plane device is but one embodiment. In some embodiments, a single two dimensional waveguide element can also be used, for example where the reaction surface is coated on one side. It may need to be oriented with the reaction surface in an upwardly facing direction, however, to facilitate contact with the sample and light scattering label reagent. Scattering of light in an evanescent wave may then be observed from the underside, e.g., using a mirror if desired.

In some embodiments, an assay chamber in combination with an assay format is archiveable. By archiveable is meant that the physical end product of the assay can be stored and results read at a later period of time. In some of these embodiments, an assay chamber is a flow cell, e.g., as shown or similar to FIG. 11. In some of these embodiments, an assay format utilizes LSLs as the detecting label. In some embodiments, the final reagent added to the assay is an oil or a glycerol. In addition, an assay chamber or flow cell can be filled at any time with fixative or any clear substance to protect the reaction sites. In some embodiments, this would include microscopy fixatives such as balsam-based or mineral oil-based compounds, or clear acrylic.

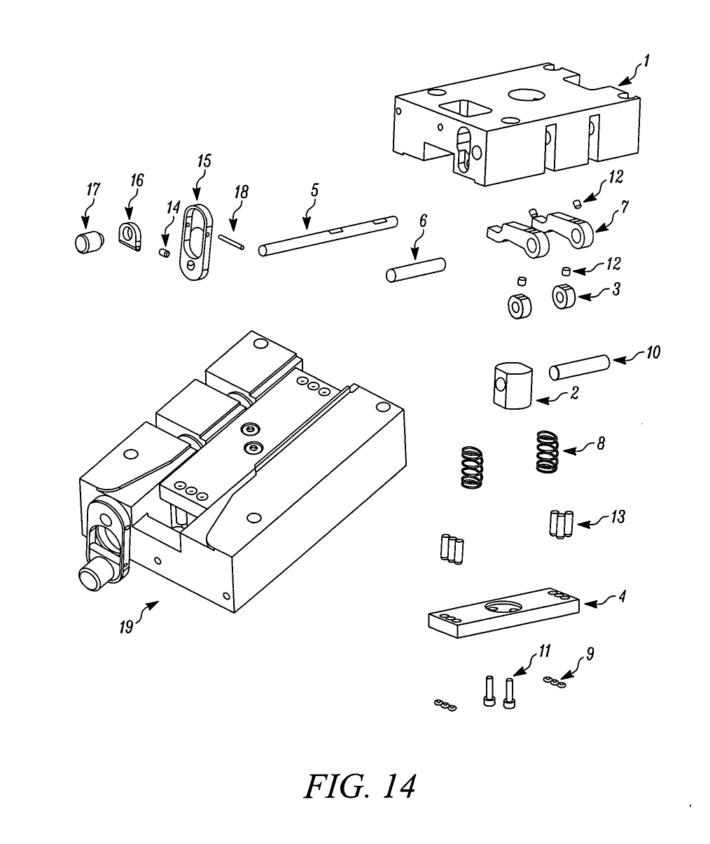

Some embodiments of the present invention provide an apparatus (e.g., a detection apparatus) wherein an assay chamber (e.g., a flow cell or waveguide device) is removable/replaceable and/or disposable. Some embodiments of the invention provide a device for holding an assay chamber of the present invention. FIG. 14 shows an exemplary embodiment of a clamping device for holding an assay chamber of the invention. This embodiment includes a slide back cover 1; a post 2; a cam 3; an o-ring plate 4; a camshaft 5 (e.g., part#51-12 from W.M. Berg, East Rockaway, N.Y.); a shaft, a lever arm (e.g., product#S1-14 from W.M. Berg) 6; an arm, lever 7; a spring, compression 8 (e.g., product#51533, Century Spring, Los Angeles, Calif.); an O-ring, as568a-002 9 (e.g., product#9452k112 McMaster-Carr, Atlanta, Ga.); a dowel pin, 0.25 inch dia×1.188 inch long, stainless steel 10; a socket head cap screw, #4-40×0.375, stainless steel 11; a set screw, cup point, #4-40×0.125 inch, stainless steel 12; a minstac fitting 13; a set screw, cup point, #4-40×0.187 inch long 14; a base, knob 15; a pivot joint, knob, a handle, knob 17; and a spring pin, 0.062 inch dia×0.625 inch long 18. In some embodiments, assembly of a device as shown in FIG. 14 comprises a press fit of component 10 into component 2. In some embodiments, assembly of a device as shown in FIG. 14 comprises applying a Loctite® adhesive for cylindrical fits to component 17 and bond to component 16. In some embodiments, assembly of a device as shown in FIG. 14 comprises a press fit of component 18 (spring pin) through the walls of component 15 to capture component 16.

In some embodiments, an assay chamber holding device is a clamp. In some embodiments, an assay chamber clamp provides a mechanical advantage using cam levers to translate rotational motion (or torque) in linear force actuation. In some embodiments, a O-ring plate (or shoe) serves as a multi-orifice (e.g., for one or more independent flow channels) seal to a flow cell superstructure and a manifold for liquid flow inlet & outlet. In some embodiments, an O-ring material is an elastomeric seal material, e.g., a Viton™ fluropolymer elastomer (DuPont Performance Elastomers LLC, Wilmington, Del.) In some embodiments, dual compression springs can be changed to vary the total compression (seal) force.

In some embodiments, a flow cell contains a marking that is read by the detection apparatus (e.g., a computer) to determine if the flow cell is properly positioned and/or inserted correctly. In some embodiments, if determined to be incorrectly positioned or inserted an audible and/or visible signal is generated. In some embodiments, if determined to be incorrectly positioned or inserted the detection apparatus will not allow the assay to be run until it detects that the flow cell or assay chamber is inserted correctly.

In some embodiments, an assay chamber is clamped into place in a detection apparatus of the present invention. In some embodiments, a detection apparatus of the present invention comprises a switch (e.g., a microswitch) which is activated or deactivated when a sample chamber (e.g., a flow cell) is inserted into and/or attached properly. In some embodiments, a clamp incorporates a microswitch to sense when the clamp is closed, and a bar code light and sensor to detect the presence of a flow cell. In some embodiments, pumps of the detection apparatus will operate only if a sample chamber (e.g., a flow cell) is properly inserted and/or the clamp is fully closed. This maintains a closed and safe system for the operator. The verification (or sensing) of a proper insertion of an assay chamber (e.g., a flow cell) could be, but is not limited to, mechanical (e.g., micro switch), magnetic (e.g., proximity or reed switch), or optical (e.g., a photo detector, a CMO, a CCD image array, or a laser reflection) verification.

In some embodiments, a sample chamber or flow cell is held in a detection apparatus using a clamp device such as depicted in FIG. 14. In some embodiments, a clamp device comprises at least one and any number of components selected from the group consisting of a flow cell clamp handle (e.g., 300 in FIG. 3) and a clamp shoe, a mobile element that presses against a flow cell and establishes (e.g., leak-proof) connections (e.g., 310 in FIG. 3), and a slot for flow cell (e.g., 320 in FIG. 3).

### Detection of Signals

As described herein, assays of the invention utilize a detectable signal, typically from a label as described herein. For example, a signal from a labeled binding molecule. Signals include, but are not limited to, optical, fluorescent, light scattering, spectroscopic, electrical, piezoelectrical, magnetic, Raman scattering, surface plasmon resonance, radiographic, calorimetric, and colorimetric methods.

In some embodiments, the detectable signal is scattered light. Scattered light may be detected, e.g., visually or by photoelectric means. For visual detection the eye and brain of an observer perform the image processing steps that result in the determination of scattering or not at a particular situs. Scattering is observed when the situs appears brighter than the surrounding background. If the number of sites is small, e.g., a dozen or less, the processing steps can be read essentially simultaneously. If the number of sites is large (a few hundred or more) a photoelectric detection systems may be a better choice.

Photoelectric detection systems include any system that uses an electrical signal which is modulated by the light intensity at a situs. Examples include, but are not limited to, a photodiode, a photodiode array, a charge coupled device, a photo transistor, a photoresistor, a photomultiplier tube, a camera, a CCD camera, a complementary metal-oxide-semiconductor (CMOS; also known as complementary-symmetry metal-oxide-semiconductor) camera or a video camera. In some embodiments, a detection system comprises a RLS scanner such as a GSD-501 RLS scanner (Invitrogen, Carlsbad, Calif.). In some embodiments of the invention, multiple detectors are arranged in an array corresponding to the array of sites on a reactive surface and optionally some detectors correspond to non-situs portions. In some embodiments, one detector (e.g., a CCD camera) is used to detect multiple situs at once. Some embodiments result in digital representations of the reactive surface such as those rendered by a charge coupled device (CCD) camera, optionally in combination with available frame grabbing and image processing software.

In some embodiments, the detector is located approximately perpendicular to an evanescent wave. In some embodiments, the detector is located approximately parallel to an evanescent wave. In some embodiments, the detector is located so as not to be parallel to an evanescent wave. In some embodiments, a CCD camera is utilized for detection of a signal, e.g., a signal resulting from a light scattering devise. In some embodiments, a Luminera 1.2 mp camera (Lumenera Corporation, Ottawa, Ontario, Canada).

In some embodiments, a detector (e.g., a CCD camera or video camera) forms an image of the reactive surface (e.g., the entire reactive surface), e.g., including all or some situs and/or all or some non-situs portions. In some embodiments, the detector detects and feeds this image to, e.g., a frame grabber card of a computer. In some embodiments, the image is converted to digital information by assigning a numerical value to each pixel. The digital system may be binary (e.g. bright=1 and dark=0). In some embodiments, a 8-bit gray scale is used, wherein a numerical value is assigned to each pixel such that a zero (0) represents a black image, and two hundred and fifty-five (255) represents a white image, the intermediate values representing various shades of gray at each pixel. Some embodiments extract and/or utilize an 8-bit or 16-bit CCD camera. Some embodiments extract and/or use a 10-bit image (e.g., .raw file) with a dynamic range from 0 to 1024 increments.

The detection and measurement of one or more detectable properties can be correlated to the presence, absence, or concentration of one or more agents in a sample. In some embodiments, a detection system optionally comprises a magnifying lens that forms a magnified image of the light scattering particle patch or a portion of the patch. In some embodiments, a magnifying lens is not utilized. In some embodiments, an illuminating system makes the label particles appear as bright objects on a dark background. In some embodiments, the number of label particles in a magnified image is quantified by particle counting. Some embodiments of the invention measure scattered light intensity (which is typically proportional to particle number or density). In some embodiments, label particle counting methods and/or detection of a signal(s) includes, but is not limited to, (a) by eye (unaided or with an ocular lens, depending on particle size), (b) an electronic imaging system (e.g., video camera, CCD camera, image intensifier) and/or (c) a photosensitive detector with a field limiting aperture and a scanning light beam arrangement. In some embodiments, a signal (e.g. scattered light intensity or fluorescence) is measured with an electronic imaging system or photosensitive detector. In some embodiments, for example at low particle surface densities (e.g., less than about 0.1 particles per μm²), a particle counting method is employed. In some embodiments, for example while at higher surface densities (especially, where the individual particles are closer than the spatial resolution capabilities of the magnifying lens), a steady light scattering intensity measurement is employed. In some embodiments, the detection apparatus is designed to easily shift between these two methods of detection, that is, between particle counting and intensity measurements.

In some embodiments, a measurement of signal is communicated to, and optionally analyzed by a computer. In some embodiments, a computer is a miniature OQO (OQO, San Francisco, Calif.). In some embodiments, a computer uses a Microsoft XP operating system (e.g., Tablet XP). In some embodiments, a computer has WiFi capability and/or USB (e.g., USB 2.0) connectivity. In some embodiments, a detection apparatus of the invention comprises a graphic user interface (GUI), e.g., see FIGS. 7 and 8.

In some embodiments, the information is displayed on a monitor, and/or stored in RAM and/or any storage device for further manipulation. In some embodiments, the digitized data file may be converted and imported into a software drawing application. This will permit printing of the image for archival purposes or analysis. Many software packages are available that will accept or convert file imports in a wide variety of file formats, including “raw”, TIFF, GIF, PCX, BMP, RLE, and many others. Typically, for printing and archival manipulations the conversions and importations should not alter the content of the data so as to result in a true and faithful representation of the image.

In some embodiments of the invention, image processing software may be used to analyze the digital information and/or determine the boundaries or contours of each situs, and/or the average or representative value of intensity at each situs. Typically the intensity of the signal correlates positively with the amount of labeled binding molecule present at the situs, and the amount of labeled binding molecule present correlates (negatively or positively, depending on the assay format) to the amount of agent at such situs.

Therefore, the present invention provides methods and compositions for acquiring, detecting and analyzing a signal(s) from an assay or the like.

### Image Analysis and Software

The present invention provides various methods for analysis of results and detectable signals. In some embodiments, results are determined by a user observing with their eye any detectable signal and mentally and/or manually analyzing and determining a result. In some embodiments, detectable signals are acquired and/or measured using electronic detection means or devices and optionally this data is analyzed via a computer and/or software.

Numerous versions of image analysis software are known in the art that are compatible with the embodiments of the invention as described herein. In some embodiments, analysis software performs qualitative and/or quantitative analysis. In some embodiments, analysis software performs, but is not limited to, slide-to-slide, assay chamber-to-assay chamber, situs-to-situs or sample-to-sample linear normalization. In some embodiments, analysis software performs artifact pixel removal and/or floor and ceiling pixel removal. In some embodiments, analysis software performs standard deviation reflecting outlier rejection. In some embodiments, the analysis of data comprises the use of a spreadsheet software such as Microsoft Excel.

For some embodiments of the invention using imaging detectors, computer software is used to identify and/or quantify an agent(s). In some embodiments, software may correct for illumination non-uniformity. In some embodiments, or if necessary, software may correct for fluorescence cross-talk through a deconvolution matrix. In some embodiments, or if necessary, software may align images using registration marks imprinted on a substrate, reactive surface, or assay chamber. In some embodiments, software may perform algorithms to distinguish agents from other signals. In some embodiments, software and/or a user may assign an identity to each imaged agent in a sample. In some embodiments, software may calculate a total number of agents in each category. In some embodiments, software may image and record a bar code for sample identification and/or for assay parameters. In some embodiments, software may automatically save output data (e.g., internal standard and/or sample data), images, and/or a bar code(s) to a database(s), e.g., that can be queried via a web browser interface. Commercially available image analysis packages can be used to provide these functions. Software packages for multicolor image analysis that can be used include, but are not limited to, Image-Pro or Image-Pro Plus (Media Cybernetics, Silver Spring, Md.); MetaMorph, e.g., version 7 (Molecular Devices, Sunnyvale, Calif.); or MatLab (The Mathworks, Inc, Natick, Mass.). In some embodiments, ArrayVision™ RLS available from Invitrogen, Carlsbad, Calif. is utilized for analysis.

Some embodiments of the invention allow multiple images of the same situs to be accumulated and analyzed over time. In some embodiments for repetitive images (e.g., of a waveguide or TIR element), illumination can occur multiple times or the lamp simply remains on until images are made at each desired time. In some cases, this will depend on the type of label. For example, time points may be preferred where the label is susceptible to photobleaching. In some embodiments, light scattering at a first time t1 is compared with scattering at a second time t2, e.g., to obtain kinetic information. This kinetic information can be valuable especially when the assay is intended to be quantitative, since the time-dependency (i.e. rate) of the increase or decrease in the amount of light scattering may be more accurately indicative of the levels of the binding pair members present in the sample than the total amount of scatter by the reaction at any given reaction point in time. Additionally, positive samples may be termed earlier than using just an endpoint assay. The use of multiple images can provide a data set over which the increase in scattered light detected is of a known function with respect to time. Measuring the rate of change of the intensity of scattered light from a given situs or region versus time provides a reaction rate. By using reaction kinetics, the rate can be correlated to a quantitative measure of agent concentration in the sample. In some embodiments, data is gathered at more than two times. Typically, the more data points obtained, the more reliable the kinetic or rate information. Therefore, the invention also provides methods and compositions for measuring reaction kinetics.

An alternative method may be used instead of reaction kinetics. In this method one integrates the detectable signal (e.g., scattered light intensity) versus time. The area obtained by this integration typically correlates to the concentration of the detected agent in a solution.

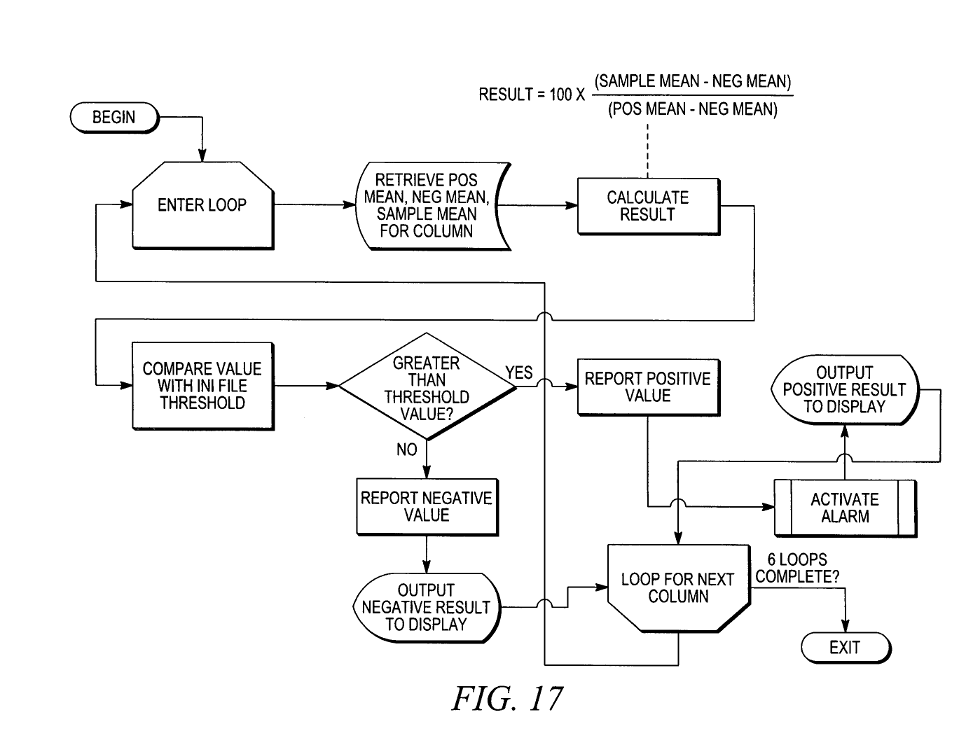

FIG. 17 shows an exemplary method and procedure for sample analysis and/or calculating results. In this exemplary method, a result for a sample is placed as a percentage of the distance between the negative and positive control pixel intensities. For example, a sample with equal intensity to the positive control would score 100. The flow chart in FIG. 17 shows that values in an “.ini” file can be used to establish minimum positive thresholds, maximum negative values, and/or the percentage used to identify a positive. These empirically-determined values can be used to minimize false positives, and/or to increase sensitivity at the expense of increased false positives.

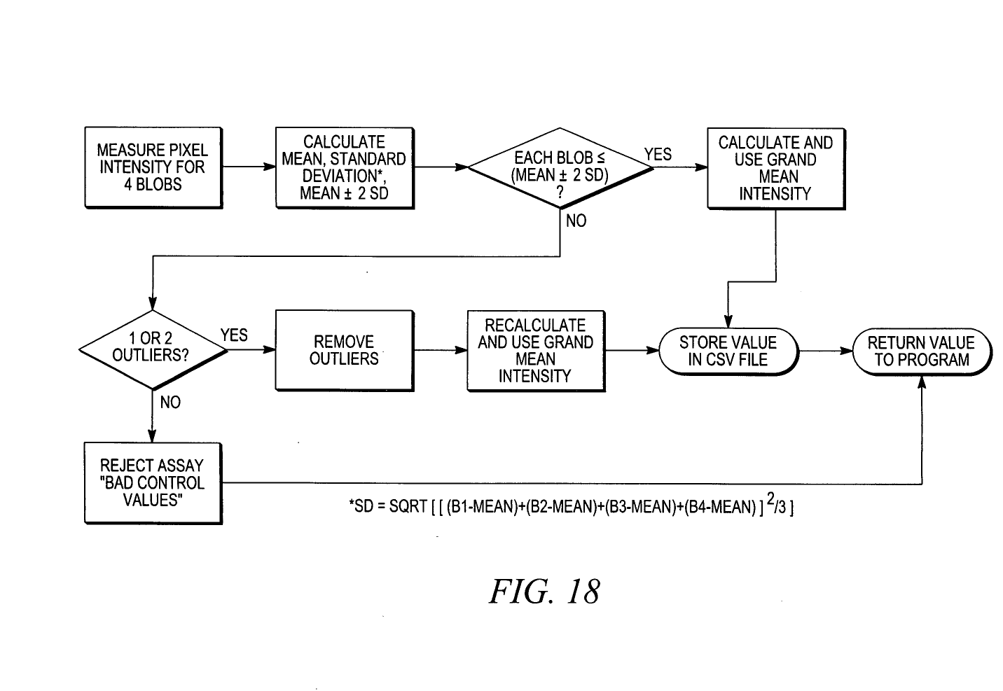

FIG. 18 shows an exemplary subroutine for blob inclusion/rejection. In some embodiments, each array feature (“blob”) is assessed for pixel intensity. The flow chart in FIG. 18 shows that outliers can be eliminated statistically from consideration, if desired. This can be important to accommodate misprinted or faulty array features.

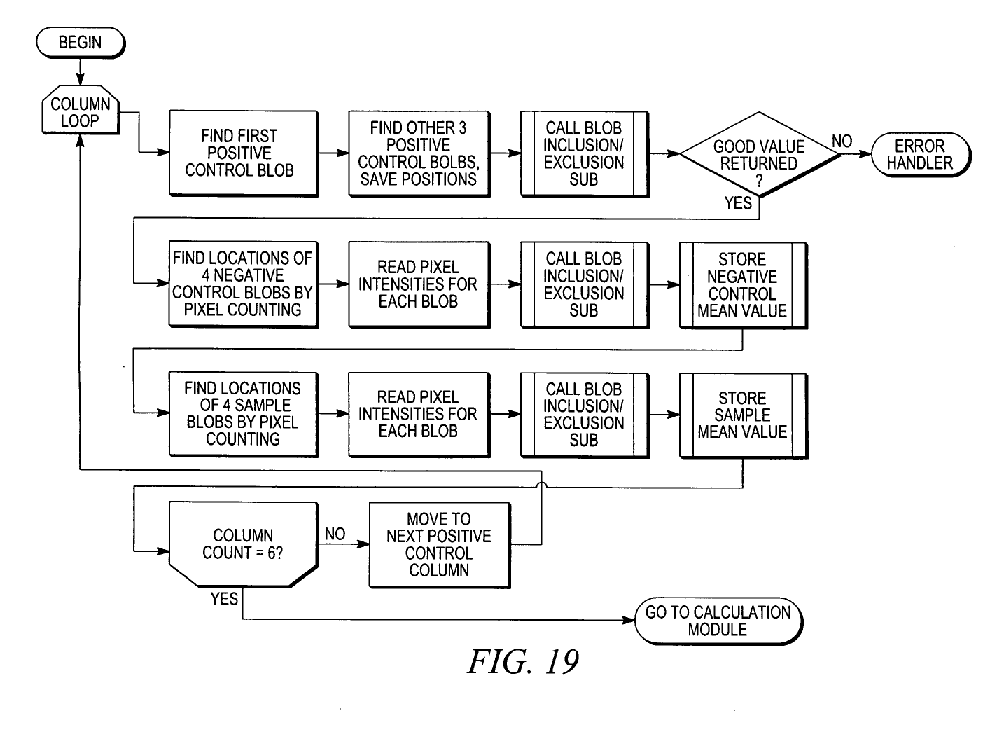

FIG. 19 shows an exemplary method for blob mean pixel intensity acquisition. The flow chart shows a method for systematically examining array features in a positive control, negative control and sample for each of six analytes in the current configuration as an example. Values can then be used to calculate the positivity or negativity of the sample.

### Light Source or Illuminator

Some embodiments of the invention utilize light and or a light source. Some embodiments, utilize a detectable label that produces a colorimetric signal which typically uses light to detect the colorimetric signal. Some embodiments, utilize a detectable label that utilizes light to produce a detectable signal. For example, a wavelength(s) of light is used to excite a label, wherein the excited label emits light at a detectable wavelength. In some embodiments, a LSL is utilized which typically requires a light source as described further herein.

A light source for generating a light beam for use in accordance with the present invention may be nearly any source of light or electromagnetic energy, including, but not limited to, energy in the visible, ultraviolet, and near-IR spectra. Of course a lights source(s) or an illuminator(s) will be compatible with a detection method(s) being employed in a particular assay. The term “light” is construed broadly herein and is not confined to the visible range. In some embodiments, non-visible wavelengths are detected by detectors optimized for the particular wavelength. The light may be, but is not limited to, monochromatic, polychromatic, collimated, uncollimated, polarized, or unpolarized light. The illumination light can be, but is not limited to, steady-state or pulsed; coherent or not coherent; polarized or unpolarized; or one, two or more different wavelengths (e.g., from the same light source or from two or more different light sources). Some embodiments of the invention utilize light sources including, but not limited to, a laser, a light emitting diode, a flash lamp, an arc lamp, an incandescent lamp, an ultracondenser (e.g., from Zeiss (Thornwood, NY)), a low wattage helium-neon laser, a laser diode, a tungsten filament bulb, a white light-emitting diode (e.g., 5 watt), a fiber lite (e.g., Bausch and Lomb), an incandescent light bulb, a Xenon arc lamp (e.g., 1000 W, ModelA-6000, Photon Technology Incorporated, Monmouth Junction, N.J.), fluorescent discharge lamps, natural visible light sources, a burning candle, igniting gas, a light stick (e.g., firefly luciferase) and/or the sun. In some embodiments of the invention, a portable disposable light source, such as those described herein, is utilized and in these embodiments the light source can optionally be a small incandescent light bulb, e.g., powered by a battery such as is used in a pocket flashlight. In some embodiments, a light source includes potentiometer means for varying the intensity of the light source. In some embodiments, filters and/or lenses may be employed to adjust the intensity to a suitable level. In some embodiments, a light source is not a laser. In some embodiments, a light source is not a UV light. In some embodiments, filters are used to allow only particular ranges of wavelengths.

In some embodiments, the light is collimated by a special lens and collected by a fiber optic bundle. In some embodiments, this bundle is a cable of over 5000 individual optical fibers, e.g., that carry the light with little loss. In some embodiments, at the end of the fiber bundle, the fibers are fanned into a line array, e.g., an aperture 1 inch long by 1/1000 inch high. In some embodiments, this light illuminates a glass slide test array through the edge of the slide, using the slide device as a waveguide.

Detection means for determining the degree of light scattering of the present invention may comprise both instrument and visual means. In some embodiments, detectable events across a reactive surface and/or assay chamber (e.g., light scattering events across the entire waveguide) can be monitored essentially simultaneously, whether by the eye and brain of an observer or by photodetection devices including, e.g., CCD cameras forming images that are digitized and processed using computers/software. In some embodiments, an illuminating system is utilized to illuminate an individual label, a group of labels, a situs or group of sites with light in such a manner that they appear as bright objects on a darker background. This allows visualization of particles attached to a surface or free in a fluid film above the surface. In some embodiments, free particles can be distinguished from attached particles by their Brownian motion which is absent in attached particles.

In some embodiments of the invention, the illuminating system is designed to (1) deliver a beam of light to a situs (or group of sites) and/or (2) minimize the amount of the illuminating light that enters the detecting system directly or through reflections. In some embodiments, this can be achieved by constraining the light beam and its reflections to angles that are outside the light collecting angles of the detecting system. In one illumination method, the collecting lens and the light source are on opposite sides of a solid-phase surface. In other embodiments, the illuminating light source and magnifying lens are on the same side of the surface.

### Kits

The present invention also provides various kits related to the assays and detection apparatuses of the invention as described herein. In some embodiments, kits may include one or more of the following: an assay reagent, combinations of assay reagents, all necessary reagents for an assay, a sample buffer, a wash buffer, a decontamination liquid or buffer, a labeled binding molecule(s), an unlabeled binding molecule(s), a control reagent(s) (e.g., positive and/or negative control samples), a reagent pack, a cleaning and/or disinfecting pack, an assay chamber (e.g., interchangeable), a detection apparatus, a manual, instructions, personal protective gear (such as gloves, a suit (e.g., Tyvek® suit), a respirator, a self contained breathing apparatus, safety glasses), software, sample collection containers (e.g., tubes, boxes, syringes), or a syringe (e.g., for inputting a sample into an assay chamber or detection apparatus). Some kits comprise at least one assay chamber (e.g., a flow cell) and at least one corresponding assay reagent, e.g. a detection binding molecule and/or a labeled binding molecule. In some embodiments, a kit comprises an assay chamber and all of the necessary reagents for performing the assay, optionally the reagents can be in a concentrated or dry (e.g., lyophilized form), for example requiring only reconstitution and/or dilution by a user and/or by the apparatus. In some embodiments related to kits comprising reagents, some or all of the reagents in the kit can be in the form of a reagent pack or packs that can be directly placed in a detection apparatus.

### Business Methods

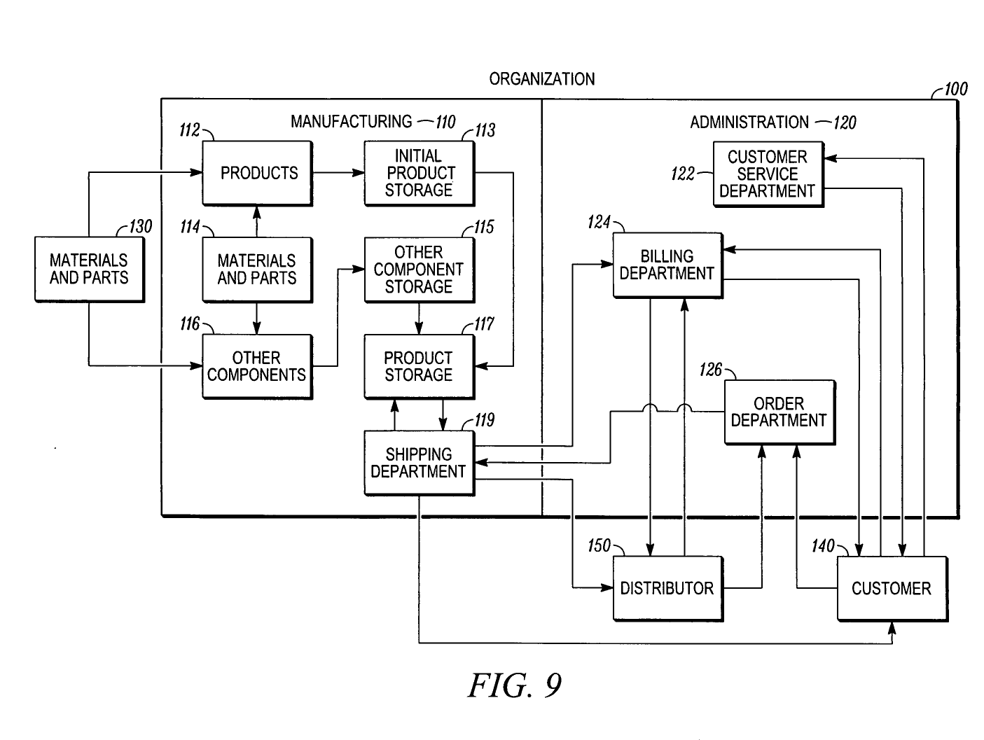

The present disclosure also provides systems and methods of providing company products to an acquirer of the products, for example, systems and methods for providing a customer or a product distributor a product such as 1) a reagent(s) related to an assay of the invention; 2) a component of a detection apparatus of the invention; or 3) a detection apparatus of the invention. FIG. 9 provides a schematic diagram of a product management system. In practice, the blocks in FIG. 9 can represent any organization which can be one entity (e.g., a legal entity) or a combination of entities that provides products or systems as disclosed herein. This organization can include departments in a single building or in different buildings, a computer program or suite of programs maintained by one or more computers, a group of employees or contractors, a computer I/O device such as a printer or fax machine, a third party entity or company that is otherwise unaffiliated with the company, or the like.

The product management system as shown in FIG. 9 is exemplified by organization 100, which receives input in the form of an order from a product or system acquirer, e.g., distributor 150 or customer 140 or the like, to order department 126, or in the form of materials and parts 130 from an acquirer; and provides output in the form of a product delivered from shipping department 119 to distributor 150 or customer 140. Organization 100 system is organized to optimize receipt of orders and delivery of products (e.g., those described herein) to a party outside of the company, e.g., in a cost efficient manner, and to obtain payment either directly or indirectly, for such product.

With respect to methods of the present disclosure, the term “materials and parts” refers to items that are used to make and package a product or other component that organization 100 sells to an acquirer. As such, materials and parts include, for example, buffers, paper, ink, reaction vessels, plastic, glass, filters, metal, assay reagents, binding molecules, pumps, light source, computer, etc. In comparison, the terms “other components” and “products” refer to items sold or otherwise supplied by the organization. Other components are exemplified by labels, covers, bottles, collars, and sleeves. As such, it will be recognized that an item useful as materials and parts as defined herein further can be considered an “other component”, which can be provided by the organization. Thus, the term “products” refers to materials and parts as well as other components that are sold or desired to be sold or otherwise provided by an organization to one or more acquirers or users.

Referring to FIG. 9, organization 100 includes manufacturing 110 and administration 120. Products 112 and other components 116 are produced in manufacturing 110, and can be stored separately therein such as in initial product storage 113 or product storage 117 and other component storage 115, respectively. Materials and parts 130 can be provided to organization 100 from an outside source and/or materials and parts 114 can be prepared by organization, and used to produce products 112 and other components 116, which, in turn, can be assembled and sold or otherwise supplied as a product. Manufacturing 110 also includes shipping department 119, which, upon receiving input as to an order, can obtain products to be shipped from product storage 117 and forward the product to a party outside the company. For example, upon receiving input from order department 126 that a customer 140 has ordered, for example, a detection apparatus, shipping department 119 can obtain from product storage 117 this product and ship the product to customer 140 (and providing input to billing department 124 that the product was shipped).

As further exemplified in FIG. 9, administration 120 includes order department 126, which receives input in the form of an order for a product from customer 140 or distributor 150. Order department 126 then provides output in the form of instructions to shipping department 119 to fill the order (e.g., to forward products as requested to customer 140 or distributor 150). Shipping department 119, in addition to filling the order, may further provide input to billing department 124, e.g., in the form of a confirmation that the products have been shipped. Billing department 124 then can provide output in the form of a bill to customer 140 or distributor 150 or other acquirer as appropriate, and can in certain embodiments further receive input that the bill has been paid, or, if no such input is received, can further provide output to customer 140 or distributor 150 that such payment may be delinquent.

An additional optional component of organization 100 includes a customer service department 122, which can receive input from customer 140 and can provide output in the form of feedback or information to customer 140. Furthermore, although not shown in FIG. 9, customer service 122 can receive input or provide output to any other component of organization. For example, customer service department 122 can receive input from customer 140 indicating that an ordered product was not received, wherein customer service department 122 can provide output to shipping department 119 and/or order department 126 and/or billing department 124 regarding the missing product, thus providing a means to assure customer 140 satisfaction. Customer service department 122 also can receive input from customer 140 in the form of requested technical information, for example, for confirming that instructions of the disclosure can be applied to the particular need of customer 140, and can provide output to customer 140 in the form of a response to the requested technical information.

As such, the components of organization 100 are suitably configured to communicate with each other to facilitate the transfer of materials and parts, other components, products, and information within organization 100, and organization 100 is further suitably configured to receive input from or provide output to an outside party. For example, a physical path can be utilized to transfer products from product storage 117 to shipping department 119 upon receiving suitable input from order department 126. Order department 126, in comparison, can be linked electronically with other components within organization 100, for example, by a communication network such as an intranet, and can be further configured to receive input, for example, from customer 140 by a telephone network, by mail or other carrier service, or via the internet. For electronic input and/or output, a direct electronic link, such as a T1 line or a direct wireless connection, can be established, particularly within organization 100 and, if desired, with distributor 150 or materials or parts provider 130, or the like.

Although not illustrated, organization 100 may have one or more data collection systems, including, for example, a customer data collection system, which can be realized as a personal computer, a computer network, a personal digital assistant (PDA), an audio recording medium, a document in which written entries are made, any suitable device capable of receiving data, or any combination of the foregoing. Data collection systems can be used to gather data associated with a customer 140 or distributor 150, including, for example, a customer's shipping address and billing address, as well as more specific information such as a customer's or other acquirer's ordering history and payment history, such data being useful, for example, to determine that the acquirer has made sufficient purchases to qualify for a discount on one or more future purchases.

Organization 100 can utilize a number of software applications to provide components of organization 100 with information or to provide a product or system acquirer access to one or more components of organization 100, for example, access to order department 126 or customer service department 122. Such software applications can comprise a communication network such as the internet, a local area network, or an intranet. For example, in an internet-based application, a customer 140 can access a suitable web site and/or a web server that cooperates with order department 126 such that customer 140 can provide input in the form of an order to order department 126. In response, order department 126 can communicate with customer 140 to confirm that the order has been received, and can further communicate with shipping department 119, providing input that products should be shipped to customer 140. In this manner, the business of organization 100 can proceed in an efficient manner.

In a networked arrangement, billing department 124 and shipping department 119, for example, can communicate with one another by way of respective computer systems. As used herein, the term “computer system” refers to general purpose computer systems such as network servers, laptop systems, desktop systems, handheld systems, personal digital assistants, computing kiosks, and the like. Similarly, in accordance with known techniques, distributor 150 can access a web site maintained by organization 100 after establishing an online connection to the network, particularly to order department 126, and can provide input in the form of an order. If desired, a hard copy of an order placed with order department 126 can be printed from the web browser application resident at distributor 150.

Various software modules associated with implementation of the present disclosure can be suitably loaded into the computer systems resident at organization 100 and any acquirer as desired, or the software code can be stored on a computer-readable medium such as a floppy disk, magnetic tape, or an optical disk. In an online implementation, a server and web site maintained by organization 100 can be configured to provide software downloads to remote users such as distributor 150, materials and parts 130, and the like. When implemented in software, the techniques of the present disclosure are carried out by code segments and instructions associated with the various process tasks described herein.

Thus, methods for selling or supplying products to such parties are provided, as are methods related to sales or supplies, including customer support, billing, product inventory management within the organization, etc. Examples of such methods are shown in FIG. 9, including, for example, wherein materials and parts 130 can be acquired from a source outside of organization 100 (e.g., a supplier) and used to prepare products, which can be maintained as an inventory in product storage 117. The other components 116 can be obtained from a source outside of organization 100 (materials and parts 130) or can be prepared within organization 100 (materials and parts 114). As such, the term “product” is used generally herein to refer an item sent to an acquirer of the product (a customer, a distributor, etc.).

At the appropriate time, the product is removed from product storage 117, for example, by shipping department 119, and sent to a requesting party such as customer 140 or distributor 150. Typically, such shipping occurs in response to the acquirer placing an order, which is then forwarded within the organization as exemplified in FIG. 9, and results in the ordered product being sent to the acquirer. Data regarding shipment of the product to the party is transmitted further within the organization, for example, from shipping department 119 to billing department 124, which, in turn, can transmit a bill to the acquirer, either with the product, or at a time after the product has been sent. Further, a bill can be sent in instances where the acquirer has not paid for the product shipped within a certain period of time (e.g., within 30 days, within 45 days, within 60 days, within 90 days, within 120 days, within from 30 days to 120 days, within from 45 days to 120 days, within from 60 days to 120 days, within from 90 days to 120 days, within from 30 days to 90 days, within from 30 days to 60 days, within from 30 days to 45 days, within from 60 days to 90 days, or during a similar time period). Typically, billing department 124 also is responsible for processing payment(s) made by the acquirer. It will be recognized that variations from the exemplified method can be utilized; for example, customer service department 122 can receive an order from the acquirer, and transmit the order to shipping department 119 (not shown), thus serving the functions exemplified in FIG. 9 by order department 126 and the customer service department 122.

Methods of the disclosure also include providing technical service to those using a product. While such a function can be performed by individuals involved in product research and development, inquiries related to technical service generally are handled, routed, and/or directed by an administrative department of the organization (e.g., customer service department 122). Often communications related to technical service (e.g., solving problems related to use of the product or individual components of the product) require a two way exchange of information, as exemplified by arrows indicating pathways of communication between customer 140 and customer service department 122.

As mentioned above, any number of variations of the process exemplified in FIG. 9 are possible and within the scope of the disclosure. Accordingly, the disclosure includes methods (e.g., business methods) that involve (1) the production of products; (2) receiving orders for these products; (3) sending the products to parties placing such orders; (4) sending bills to parties obliged to pay for products sent to such; and/or (5) receiving payment for products sent to parties. For example, methods are provided that comprise two or more of the following steps: (a) obtaining parts, materials, and/or components from a supplier; (b) preparing one or more first products; (c) storing the one or more first products of step (b); (d) combining the one or more first products of step (b) with one or more other components to form one or more second products (e.g., a detection apparatus); (e) storing the one or more first products of step (b) or one or more second products of step (d); (f) obtaining an order of a first product of step (b) or a second product of step (d); (g) shipping either the first product of step (b) or the second product of step (d) to the party that placed the order of step (f); (h) tracking data regarding the amount of money owed by the party to which the product is shipped in step (g); (i) sending a bill to the party to which the product is shipped in step (g); (j) obtaining payment for the product shipped in step (g) (generally, but not necessarily, the payment is made by the party to which the product was shipped in step (g)); and (k) exchanging technical information between the organization and a party in possession of a product shipped in step (d) (typically, the party to which the product was shipped in step (g)). For clarity, any of steps (a) to (k) are optional and can typically be omitted or carried out by another entity.

The present disclosure also provides systems and methods for providing information as to availability of a product (e.g., a detection apparatus or a component/reagent thereof) to parties having potential interest in the availability of the product. Such a method, which encompasses a method of advertising to the general or a specified public, the availability of the product can be performed, for example, by transmitting product description data to an output source, for example, an advertiser; further transmitting to the output source instructions to publish the product information data in media accessible to the potential interested parties; and detecting publication of the data in the media, thereby providing information as to availability of the product to parties having potential interest in the availability of the product.

Accordingly, the present disclosure provides methods for advertising and/or marketing devices, products, and/or methods of the disclosure, such methods providing the advantage of inducing and/or increasing the sales of such devices, products, and/or methods. For example, advertising and/or marketing methods of the disclosure include those in which technical specifications and/or descriptions of devices and/or products; methods of using the devices and/or products; and/or instructions for practicing the methods and/or using the devices and/or products are presented to potential interested parties, particularly potential purchasers of the product such as customers, distributors, and the like. In particular embodiments, the advertising and/or marketing methods involve presenting such information in a tangible or an intangible form to the potential interested parties. As disclosed herein and well known in the art, the term “intangible form” means a form that cannot be physically handled and includes, for example, electronic media (e.g., e-mail, internet web pages, etc.), broadcasts (e.g., television, radio, etc.), and direct contacts (e.g., telephone calls between individuals, between automated machines and individuals, between machines, etc.); whereas the term “tangible form” means a form that can be physically handled.

The disclosure further provides methods associated with the design of custom products. These methods include, for example, (1) the taking an order from a customer, e.g., for a detection apparatus for detecting a particular agent or agents, (2) preparation of detection apparatuses, (3) and providing (e.g., shipping) the product to the customer. Additionally, in particular embodiments, the customer may be billed for the detection apparatus with the bill either being sent to the customer along with the medium or sent separately.

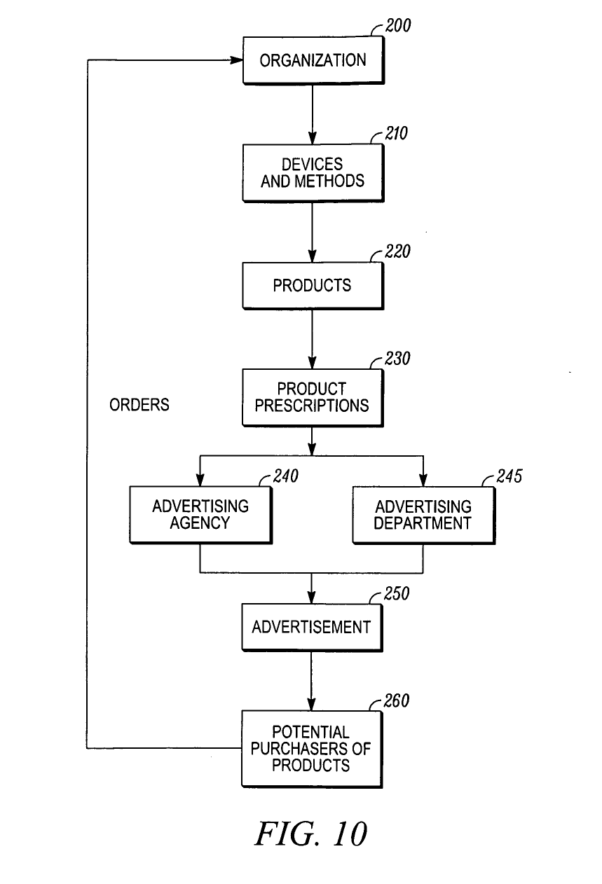

FIG. 10 provides a schematic diagram of an information-providing management system as encompassed within the present disclosure. In practice, the blocks in FIG. 10 can represent any organization which can be one legal entity or a combination of entities that provide products or systems as disclosed herein, which can include departments in a single building or in different buildings, a computer program or suite of programs maintained by one or more computers, a group of employees or contractors, a computer I/O device such as a printer or fax machine, a third party entity or company that is otherwise unaffiliated with the company, or the like.

The information-providing management system as shown in FIG. 10 is exemplified by organization 200, which makes, purchases, or otherwise makes available that organization 200 wishes to sell to interested parties. To this end, product descriptions 230 may be made, providing information that would lead potential users to believe that products 220 can be useful to user or other acquirer. In order to effect transfer of product descriptions 230 to the potential users or other acquirers, product descriptions 230 may be provided to advertising agency 240, which can be an entity separate from organization 200, or to advertising department 245, which can be an entity related to organization 200, for example, a subsidiary. Based on the product descriptions 230, advertisement 250 is generated and is provided to media accessible to potential purchasers of products 260, who may then contact organization 200 to purchase products 220.

By way of example, product descriptions 230 can be in a tangible form such as written descriptions, which can be delivered (e.g., mailed, couriered, etc.) to advertising agency 240 and/or advertising department 245, or can be in an intangible form such as entered into and stored in a database (e.g., on a computer, in an electronic media, etc.) and transmitted to advertising agency 240 and/or advertising department 245 over a telephone line, T1 line, wireless network, internet, intranet, or the like. Similarly, advertisement 250 can be a tangible or intangible form such that it conveniently and effectively can be provided to potential parties of interest (e.g., potential purchasers of product 260). For example, advertisement 250 can be provided in printed form as flyers (e.g., at a meeting or other congregation of potential interested parties) or as printed pages (or portions thereof) in magazines known to be read by the potential interested parties (e.g., trade magazines, journals, newspapers, etc.). In addition, or alternatively, advertisement 250 can be provided in the form of directed mailing of computer media containing the advertisement (e.g., CDs, DVDs, floppy discs, etc.) or of e-mail (e.g., mail or e-mail that is sent only to selected parties, for example, parties known to members of an organization that includes or is likely to include potential users or other acquirers of products 220); of web pages (e.g., on a website provided by organization 200, or having links to the organization 200 website); or of pop-up or pop-under ads on web pages known to be visited by potential purchaser of products 260, and the like. Potential purchasers or other acquirers of products 260, upon being apprised of the availability of the products 220, if so desired, can then contact organization 200 and can order the products 220 from organization 200 (see FIG. 9).

Also provided are methods for advertising which are designed to (1) result in increased sales, (2) to result in increased numbers of customers which use one or more products, and/or (3) to affect choices by potential customers which result in the selection of one product over another by the potential customers. These methods may include, for example, describing features of a product of the disclosure. In many instances, advertising methods of the disclosure will be in tangible form, such as flyers (e.g., brochures or cards suitable for mailing, posters presented at trade shows, etc.), newsletters, print advertisement in periodicals (e.g., newspapers, magazines, etc.).

The disclosure further includes advertisements themselves. Thus, the disclosure includes, for example, a composition comprising a full page or partial page advertisement in a magazine (e.g., a trade related magazine such as Science, Biochemistry, The Journal of Molecular Biology, Virology, etc.) in which features of products with one or more aspects of the disclosure are presented and/or compared to one or more additional products. These one or more additional products may be available from the same supplier, different suppliers, or the combination of the same supplier and one or more different suppliers. When a supplier of the products with one or more aspect of the disclosure provides a comparison with a product from the same supplier, the advertisement will often be designed to present new features of the products with one or more aspect of the disclosure to educate potential customers. In some instances, comparisons between different products will be in graphic form (e.g., photographs, charts, tables, etc.).

The disclosure also includes methods for performing comparative studies between products and/or product format (e.g., comparing a detection method and/or reagent of the invention to another type of detection method and/or reagent). These comparative studies may include, for example, (1) providing one or more products with one or more aspects of the present disclosure to one or more sets of users (e.g., people who use products of the kind), (2) use of the provided product(s) by the users, (3) receiving data related to the opinion of users regarding the provided product(s), and optionally (4) assessing the data received to determine the results of the comparison. Comparative studies may or may not include providing and/or use of additional products (e.g., products to which products with one or more aspect of the disclosure are to be compared) by users who are to provide the data referred to above in step (3).

In many instances, it will not be necessary for users to actually use additional products at the same time as one or more products with one or more aspect of the disclosure. This is so because, for example, many users will be familiar with the additional products. Also, in instances where a comparative study is to be done, e.g., with users who are not familiar with the additional products, a “blind” study can be performed. In other words, comparative studies can be performed by different users who each supply data related to different products.

The disclosure also includes methods for increasing market share for particular product items or product categories. In particular, methods of the disclosure include those in which products with one or more aspect of the disclosure (1) are brought to the attention of potential customers and (2) a particular percentage of the potential customers who previously purchased other products (e.g., products lacking some or all aspects of the disclosure) begin purchasing products with one or more aspect of the disclosure instead of the other products. The percentage of potential customers who switch from purchasing other products to purchasing products with one or more aspect of the disclosure may vary greatly but may be between 1% and 10%, 1% and 20%, 1% and 30%, 1% and 40%, 1% and 60%, 1% and 80%, 1% and 100%, 10% and 20%, 10% and 40%, 10% and 50%, 10% and 70%, 10% and 85%, 10% and 100%, 30% and 60%, 30% and 80%, 30% and 100%, 40% and 60%, 40% and 80%, 40% and 100%, 50% and 70%, 50% and 90%, 50% and 100%, etc. In some aspects, the percentage of potential customers who switch from purchasing other products to purchasing products with one or more aspect of the disclosure may be greater than 2%, greater than 5%, greater than 10%, greater than 20%, greater than 30%, greater than 40%, greater than 50%, greater than 65%, greater than 75%, greater than 85%, etc. The percentage of potential customers who switch form purchasing other products to products with one or more aspect of the disclosure may be determined by any means known in the art, and can be determined in certain embodiments by survey. In this method a representative number of users or former users of the products are asked to complete a survey with questions designed to determine the amount of use of the products prior to, and after having seen the advertisement. The number of customers or potential customers who have begun or stopped using any particular product or group of products can then be determined.

Another method for determining product acceptance and/or increase in market share is by, for example, the number of parties which switch from purchasing one product to purchasing another product such as a product of the present invention.

In certain aspects, the method is a method for generating revenue by providing a purchasing function to a customer to purchase a product or service provided herein. For example, the purchasing function can include providing a telephonic ordering system, a direct sales representative, or by utilizing a computer system that displays a visual representation on a monitor, of a link to purchase a product or service disclosed herein. The method can further include providing a computer-based ordering function that is activated when the visual representation is selected.

All publications, patents and patent applications mentioned in this specification are herein incorporated by reference in their entirety into the specification to the same extent as if each individual publication, patent or patent application was specifically and individually indicated to be incorporated herein by reference.

### 6. EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

Whereas, particular embodiments of the invention have been described herein for purposes of description, it will be appreciated by those skilled in the art that numerous variations of the details may be made without departing from the invention as described in the appended claims.

The experimental results shown in the following examples are shown as examples for proof of concept. The similar results could be obtained and/or read using a detection apparatus of the invention.

### Example 1

### Exemplary Antigens and Antibodies

Some agents utilized in related experiments included: B. anthracis Protective Antigen (PA); B. globigii, a simulant for gram-positive bacteria; Staphylococcal enterotoxin B; C. botulinum toxoid A; Y. pestis; and Ricin A chain. Most agents and antibodies used were kindly supplied by the Critical Reagents Program (CRP) of the Department of Defense (DOD). For results from some field assays, agents were supplied by the DOD and in some cases were not inactivated.

The antibodies above can be utilized as capture and/or detector binding molecules for the above agents. These include the following antibodies from the CRP: anti-anthrax E-062303, anti-B. globigii J-290501-03, anti-SEB 060299-01, anti-Botulinum toxin J-280800-01, anti-Y. pestis N-190803-01, and anti-ricin R-1054. In some cases, the same antibody is used as the detector and capture antibody, except that the detector antibody may comprise a label or tag.

### Example 2

### An Example of Threat Detection Sensitivity and Specificity Data Using an RLS Assay

The following results were obtained using a prototype assay and assay chamber. Testing was performed on government furnished samples without knowledge of their content. All samples represented live organisms and active toxins. Testing was performed under ‘field conditions’ in a trailer at the U.S. Government's Dugway Proving Ground in Utah.

1,074 data points were collected from 358 samples over 12 days of testing resulting in one false positive (0.09%). Results are shown in Table 2

TABLE 2

| Anthrax | | | BoToxA | | | Yersinia | | | | | |
|---|---|---|---|---|---|---|---|---|---|---|---|
| (CFU/ml) | | | ng/ml | | | CFU/ml | | | Blank | | |
| 10⁶ | 26/26 | 100% | 10² | 24/24 | 100% | 10⁵ | 24/24 | 100% | — | 29/29 | 100% |
| 10⁵ | 35/35 | 100% | 10¹ | 29/32 | 91% | 10⁴ | 29/29 | 100% | | | |
| 10⁴ | 26/33 | 79% | 10⁰ | 23/26 | 88% | 10³ | 24/30 | 80% | | | |
| 10³ | 0/23 | 0% | 10⁻¹ | 5/21 | 24% | 10² | 0/21 | 0% | | | |

### Example 3

### Examples of Readouts from Different Array Configurations

An array of spots/sites of capture antibody (anti B. anthracis) is placed on a substrate/reactive surface using a Biodot spotter (BioDot, Inc, Irvine, Calif.). Spotting is typically performed in volumes between about 10 nanoliters (nl) to 20 nl) Typically, the capture antibodies are spotted in a carbonate buffer such as sodium bicarbonate or calcium bicarbonate. In some cases, a spotting solution comprises dimethyl sulfoxide (DMSO), e.g., at 1%. Usually the carbonate buffer is of an acidic pH, e.g., about 8 to about 9, about 9 to about 10, about 10 to about 11, about 11 to about 12, about 12 to about 13, about 13 to about 14, or about 9.6. Spotting solutions typically contain antibodies at a concentration of between from about 1 to about 10 mg/ml.

Agent (inactivated, so as not pathogenic) is added to the spotted capture binding molecule, followed by biotinylated detector antibody and RLS gold-anti-biotin particles.

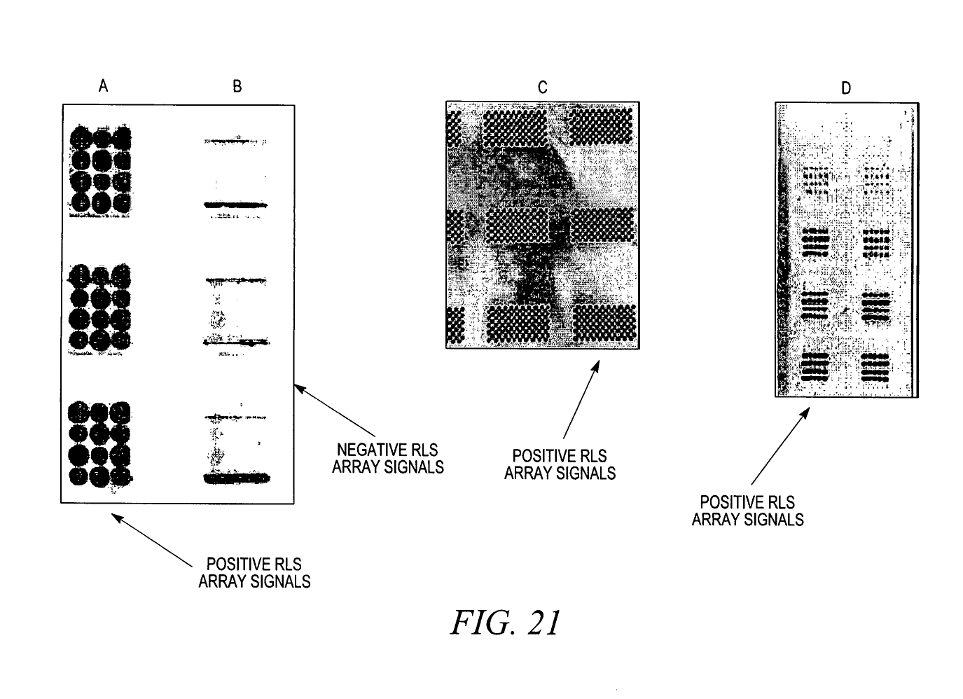

FIG. 21 shows examples of photographs of agent detection results for detection of anthrax.

### Example 4

### Real Time Monitoring of Signal Generation

In a real-time monitoring configuration (real-time RLS (rtRLS) or real-time enzyme-linked immunoSorbent assay (rtELISA)), a reaction is continuously monitored for the development of a positive response or a change in response, e.g., as conditions vary.

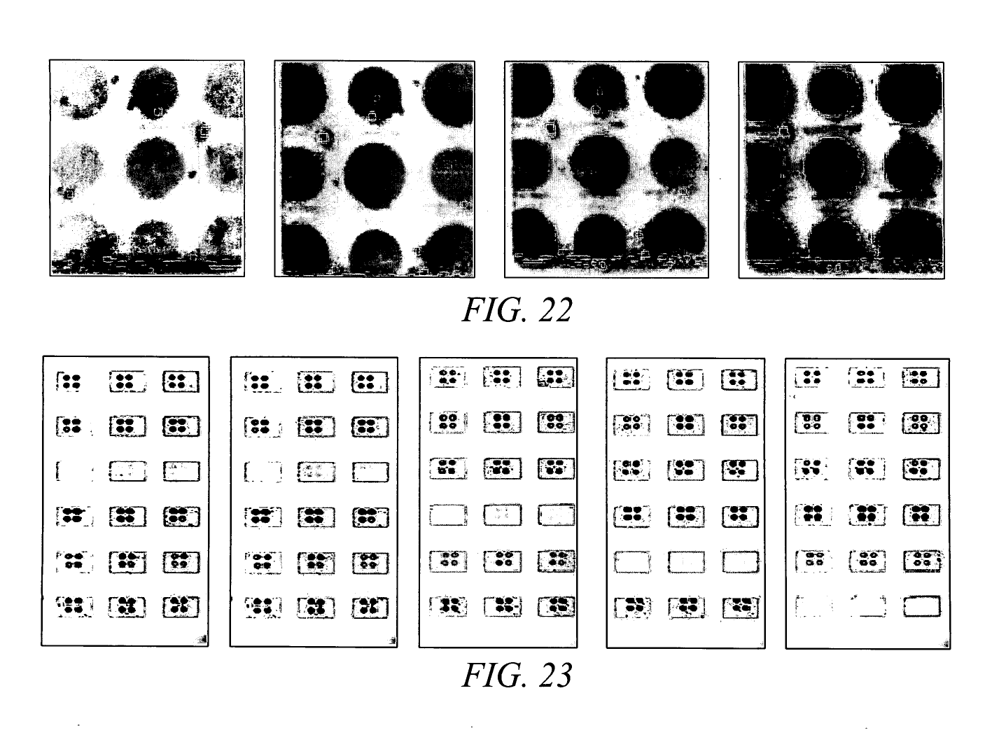

In some embodiments, when a positive is detected, it can be displayed on a computer screen. As time progresses, additional positives may appear. Since the flow cells and sites remain in the optical path during the assay, the development of RLS signal can be monitored at will, and a user notified as soon as a positive is detected. FIG. 22 shows an example of real-time or time point monitoring with increasing time from left to right.

### Example 5

### Specificity of an RLS Signal from an Assay Simultaneously Detecting Six Agents

As described herein, an assay chamber or reactive surface can be designed to detect multiple agents in one sample. FIG. 23 shows examples of RLS signals from experiments simultaneously detecting B. anthracis Protective Antigen (PA); B. globigii, a simulant for gram-positive bacteria; Staphylococcal enterotoxin B; C. botulinum toxoid A; Y. pestis; and Ricin A chain. Note for each panel in FIG. 23, one agent was not included in each assay. This type of assay chamber, reactive surface and readout is compatible with some detection apparatuses of the invention.

### Example 6

### Example of Detection of Toxins (Ricin and Botulinum Toxoid)

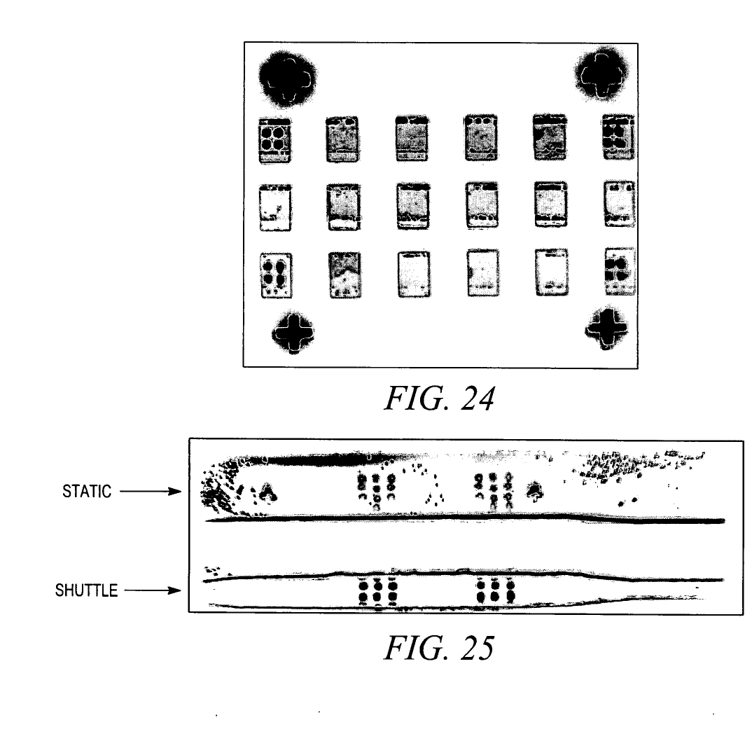

FIG. 24 shows results for the detection of ricin and botulinum toxoid. The top row shows positive results for the two toxins (at each end of the array) and negative results for all other antigen detection spots/sites (B. anthracis Protective Antigen; B. globigii; Staphylococcal enterotoxin B; and Y. pestis). The bottom row shows the positive controls for these two toxins. Positive controls were deposited and dried in the flow cell. The center row represents negative controls.

### Example 7

### Example of Sensitivity of Detection of Ricin

Example of RLS signal from experiments detecting Ricin A chain is shown in Table 3. Detection of other agents may yield similar levels of detection but may be dependent upon, inter alia, the quality of binding molecules utilized, the sample type and the agent itself

TABLE 3

| Concentration of agent | |
|---|---|
| (per ml) | Results |
| 2 ug | positive |
| 0.5 ug | positive |
| 125 ng | positive |
| 31.5 ng | positive |
| 7.81 ng | positive |
| 1.95 ng | positive/negative |
| negative | negative |

### Example 8

### Examining the Effect of Static Versus Non-Static Incubation

Optimum RLS detection of agents can be dependent upon assay methodology. FIG. 25 shows agent detection comparing movement of the reagents in a microfluidic fashion compared to a static, non-movement type of assay.

### Example 9

### Exemplary Product Literature

### MAPP-DS Pathogen Array Kit

### Shipping and Storage

The Multi-Agent Portable Pathogen Detection System (MAPP-DS) Pathogen Array Kit is shipped in a sealed foil package on blue ice. Upon receipt, store the package at 2-8° C. Do not freeze.

The expiration date is printed on the package. Use the array before the expiration date for best results.

### Description

The MAPP-DS Pathogen Array is a low-density antibody array containing capture antibodies for six targets (listed on the next page). Each capture antibody is spotted onto the aminopropylsilane-coated glass slide using an automated microarrayer.

The complete array assembly provided with each kit is shown below, and includes:
A “single-use” cell superstructure, constructed of black acrylic, with ports for fluid access.
A flow gasket, laser-cut from multi-laminate tape and coated on both sides with acrylic adhesive, which holds the cell together and creates the three flow channels (positive control, negative control, and test sample).
The aminopropylsilane-coated test slide, pre-arrayed with capture antibodies, masked with black epoxy, with 18 “windows” that are interrogated in the assay and white registration marks for the optical recognition system.
   Array Specifications The proteins on the array are printed in 18 subarrays and are equally spaced in vertical and horizontal directions. The specifications for the array are listed below:

Slide Dimensions: 1 inch×3 inch (25 mm×75 mm)

Total Subarrays: 18 (3 columns×6 rows)

Subarray Dimensions: 2 rows×2 columns

Median Spot Size: 20 picoliter (pl)

### Array Content

The capture antibodies are arrayed on the slide in triplicate. The following diagram shows the location of each capture antibody on the slide.

| | 1 | 2 | 3 | 4 | 5 | 6 |
|---|---|---|---|---|---|---|
| A | Anti-Bacillus | Anti- | Anti- | Anti- | Anti- | Anti-Ricin |
| | anthracis | Bacillus | Staphylococcal | Botulinium | Yersinia | |
| | protective antigen | globigii | enterotoxin B | toxin | pestis | |
| B | Anti-Bacillus | Anti- | Anti- | Anti- | Anti- | Anti-Ricin |
| | anthracis | Bacillus | Staphylococcal | Botulinium | Yersinia | |
| | protective antigen | globigii | enterotoxin B | toxin | pestis | |
| C | Anti-Bacillus | Anti- | Anti- | Anti- | Anti- | Anti-Ricin |
| | anthracis | Bacillus | Staphylococcal | Botulinium | Yersinia | |
| | Protective antigen | globigii | enterotoxin B | toxin | pestis | |

### Antibody Concentrations

The approximate concentration of each capture antibody on the slide is listed below:

| | Approximate |
|---|---|
| Capture Antibody | Concentration |
| Anti-Bacillus anthracis Protective antigen | 350 μg/ml |
| Anti-Bacillus globigii | 500 μg/ml |
| Anti-Staphylococcal enterotoxin B | 125 μg/ml |
| Anti-Botulinium toxin | 250 μg/ml |
| Anti-Yersinia pestis | 500 μg/ml |
| Anti-Ricin | 250 μg/ml |

### Controls

Each array assembly contains six controls that are spotted onto the cell superstructure. These controls are listed below:

| Control | Quantity per Array |
|---|---|
| Recombinant Bacillus anthracis protective antigen | 2 μg |
| Bacillus globigii spores | 3 μg |
| Staphylococcal enterotoxin B (SEB) | 45 μg |
| Clostridium botulinum Type A Complex toxoid | 5 μg |
| Ricin A chain | 1 μg |
| Inactivated Yersinia pestis | 10 μg |

### Printing Process

The purified antibodies are printed in a dust-free, temperature- and humidity-controlled environment to maintain consistent quality of the microarrays. The arrays are printed using an automated process on a microarrayer that is extensively calibrated and tested for printing MAPP-DS Pathogen Arrays. Following printing, the array is visually examined for obvious defects.

### Example 10

### Exemplary Product Literature

### MAPP-DS Reagent Pack and Cleaning Pack

### Products

This insert is supplied with the following products:

| Kit | Contents |
|---|---|
| MAPP-DS Reagent Pack | 1 tray (6 wells) |
| MAPP-DS Cleaning Pack | 1 tray (6 wells) |

### Storage

Store both the Reagent Pack and the Cleaning Pack at 2-8° C. Do not freeze.

### MAPP-DS Reagent Pack

Each Multi-Agent Portable Pathogen Detection System (MAPP-DS) Reagent Pack is a molded plastic tray consisting of 6 sealed wells containing the necessary reagents to perform one multiplex immunoassay on the MAPP-DS instrument. Wells 1-5 are filled with reagents, while well 6 contains absorptive crystals for the collection of waste materials.

The components in each well are listed below:

| | Well |
|---|---|
| Component | no. |
| Blocker (1% w/v Casein Hammersten Grade in PBS, Kathon as | 1 |
| preservative, pH 7.4) | |
| Detection antibodies - Gold labeled | 2 |
| 50% Glycerol | 3 |
| Cleaning solution (5% Bleach) | 4 |
| Wash solution (water) | 5 |
| Absorptive crystals (for collecting waste materials) | 6 |

MAPP-DS Cleaning Pack The MAPP-DS Cleaning Pack is a molded plastic tray consisting of 6 sealed wells containing the following solution for cleaning the MAPP-DS instrument:

| Reagent | Well no. |
|---|---|
| Cleaning solution (1% 5-Bromo-5 nitro-1,3-dioxane in water) | 1-6 |

### Example 11

### Multi-Agent Portable Pathogen Detection System (MAPP-DS™)

An exemplary MAPP-DS™ (developed by Invitrogen Federal Systems, Carlsbad, Calif.), for the testing of six biothreat agents (B. anthracis PA, BG (simulant), C. botulinum toxin Type A, Staphylococcal enterotoxin B, Y. pestis, and Ricin A chain) is based on Resonance Light Scattering (RLS) with the generation of signal from gold particles bound to specific antibodies. This signal, induced by white light, is detected on a capture antibody array, recorded by a digital camera, and analyzed by comparison to concurrent positive and negative controls.

The MAPP-DS™ includes an MAPP-DS™ instrument, Instruction Manual, single-use Reagent Packs, single-use Flow Cells, and maintenance items (Rinse Packs and Rinse Cells). An MAPP-DS™ assay protocol takes about 40 minutes, from start to finish, with results available as early as 24 minutes, depending upon the concentration of the agent being tested.

The reservoir packs can be single-use blister packs that contain 4 small (4 mL) reservoirs, one 15 mL reservoir, and one waste reservoir. The 4 small reservoirs contain wetting fluid (casein-based blocking solution in phosphate-buffered saline), secondary RLS reagent (gold-labeled antibody mixture), developing reagent (glycerol-based buffered saline), and decontamination solution (sodium hypochlorite). The large reservoir contains water. The reservoir packs should be stored at about 4° C.

The Cleaning Pack, can be a single-use tray filled with appropriate cleaning solutions. The front compartments contain water with, for example, 0.1% Tween-20 detergent. The smaller rear compartment contains water. All compartments are supplemented with an antimicrobial such as 0.2% Bronidox (5-Bromo-5-Nitro-1,3-Dioxane).

The flow cell is a single-use device, individually packed in a moisture-proof foil desiccant pack. It includes the positive controls for the assay, which are dried onto the plastic superstructure, under the glass slide. The flow cells are provided with a luer cap, which should remain on the cell until it is used. The flow cell should be stored at about 4° C., and not frozen.

The MAPP-DS™ Cleaning Cell is provided for instrument maintenance. The luer cap for this cell is never removed. The cell provides a fluid path for cleaning and decontaminating the MAPP-DS™ instrument. The MAPP-DS™ Cleaning Cell can be stored at room temperature.

Specifications of an Exemplary MAPP-DS ™ Apparatus

| | |
|---|---|
| Input Power: | AC 100-124 V, 50/60 Hz. Grounding required. |
| | DC, NiMH (Nickel Metal Hydride) battery, |
| | rechargeable. |
| Installation Site: | Indoor/Outdoor use. Dry environment only |
| | (not waterproof when case is open). |
| Operating | 5-40 degrees C. Unit contains an internal |
| temperature: | reagent heater. |
| Maximum Relative | 80% for temperatures up to 31 degrees C., |
| Humidity: | decreasing linearly to 50% relative |
| | humidity at 40 degrees C. |
| Instrument Type: | Portable encased unit containing computer- |
| | driven optics, fluidics and mechanical |
| | systems. |
| Sample Processing: | Accepts single, 1 ml, particle-free liquid |
| | sample, assessed for 6 pathogens simul- |
| | taneously. |
| Processing time: | Variable (see manual for details). Time to |
| | first result about 20 minutes. |
| Software: | Proprietary MAPP-DS ™ System, Microsoft |
| | Windows XP operating system. |
| Dimensions: | Pelican 1400 case; 13.37″ × 11.62″ × 6.00″ |
| | (27 × 24.6 × 17.4 cm) |
| Weight: | 19 Pounds (8.6 Kg) |

The outer case can be, for example, the Model 1400 case by Pelican Product, Inc. The case is made from a polypropylene copolymer material. When closed, it is waterproof, crush-proof and dust-proof (o-ring seal). The case is secured by two latches which pull upwards to open the hinged lid. There is an automatic pressure equalization valve to accommodate changing pressure, for example, during air transport. Two stainless steel padlock protectors are present for security. The case requires no maintenance other than cleaning with a damp cloth and household cleaner.

The top panel contains all components necessary to operate the MAPP-DS™ instrument. The device lid hinges upward, and remains open whenever the instrument is running As shown in FIG. 4, when the line cord is not used, it must be coiled and enclosed in the retainer. When the lid is closed, the two latches provide a water-tight seal for the instrument. The user may secure the instrument using the padlock/security openings molded into the cover (lock not provided).

The Tablet Computer, which provides all program control for the detection apparatus, for example can be the OQO (OQO Inc., San Francisco, Calif.), Windows XP Professional, Tablet PC. It is used in “Portrait” mode in the exemplary MAPP-DS™ instrument, but can be changed to “Landscape” mode by the user. The MAPP-DS™ assay can be run completely in the closed configuration using a special stylus. If necessary to use keyboard input, the top of the OQO slides to the left, exposing the keyboard. The mouse buttons are at the bottom (left mouse button on the left, right mouse button on the right). The mouse itself is controlled by the black finger pad at the right side of the keyboard.

### Example 12

### MAPP-DS™ Exemplary Assay Protocol

Place the MAPP-DS™ instrument on a level laboratory bench, table, or flat ground. The instrument must remain level during the testing procedure. Open the instrument cover by lifting the two latches at the front edge, and pulling up on the cover until it is in a fully open position Insure that the power switch is in the OFF position.

If operating on AC, unwind the line cord from its bracket in the case lid. Do not disengage the guide clip on the left side. Connect the cord to a grounded AC outlet, 110-120 VAC. Turn the power switch to the “AC” position. If operating on battery power, leave the line cord in its bracket. Turn the power switch to “DC”. When the battery is low, an indicator will appear on the OQO screen—“Battery Low”. If this notification is seen, the battery must be recharged as soon as possible. A full charge will operate the MAPP-DS™ for 2-4 hours.

The battery will charge when the MAPP-DS™ is attached to the AC line, and the power switch is in the “AC” position. This charging will take place whether or not the instrument is running an assay. A full charge will take approximately 14-18 hours.

Start the MAPP-DS™ Unit by turning the power switch to AC if using main power, or to DC for battery operation. Push the power button, located at the upper right edge of the computer screen, to begin the boot process. The OQO computer is started by pressing the on button, which will light when pressed, and will take approximately 3 minutes to boot from a cold start. The OQO will boot directly to the MAPP-DS™ program. During this time, anti-virus software is loaded, and machine parameters are initiated. When the MAPP-DS™ program is displayed, the instrument is ready for use. No additional warm-up is required.

All commands to the OQO must be made using the special stylus, stored in a bracket adjacent to the power cord. There is a push-button located at the bottom of the stylus. Holding this button down when touching the screen emulates a “right-click” mouse button. Tapping the screen without holding the button emulates a “left-click” or “enter” mouse button.

Prepare the sample to be tested. Fill a syringe with 1 ml aqueous sample for testing. The optimal sample volume is 1 ml however smaller volumes, down to 0.25 ml, can be used when necessary. It is preferable when the sample size is less than 1 ml to bring the sample volume to 1.0 ml using phosphate-buffered saline or a similar physiological buffer before use. The MAPP-DS™ instrument can detect agents in a sample that are soluble in aqueous phase, and do not contain particulates. It is recommended that the sample, if not free of particulates or insoluble material (particulate size larger than about 0.8 μm), is filtered prior to testing.

### Insert the Reagent Pack

The reagent pack is a single-use package, stored at 4 degrees C. and should be brought to room temperature before use (15-30 minutes).

Remove the Reagent Pack from the storage box, and invert gently about 10 times to mix the fluids. Insert the Reagent Pack into the receiver and close and latch the lid as described below.

Open the Reagent Pack Receiver by moving the handle, to the right opening the lid toward the rear, see FIG. 4.

Insert the Reagent Pack, with the foil side up and the clear plastic side down, by sliding it completely into the receiver.

Close the receiver lid, and press down with both hands. This will extend the reagent sampling pins/tubes through the foil, into the reagent compartments. The lid will lock down on both sides, and the handle will spring back to the closed position.

Insert the Flow Cell

The flow cell is supplied in a heat-sealed foil, single-use package. It should be removed from 4 degree C. storage and brought to room temperature before use (about 15-30 minutes).

Remove the Flow Cell from its foil package, open the clamp, and insert the flow cell fully into its slot in the top panel, as follows

Open the flow cell clamp lever by moving it down and toward the front of the instrument.

Remove any cell, such as the cleaning cell, still in the instrument by pulling straight upward.

Insert the flow cell completely into the receiver slot. The front of the flow cell (masked side) should face toward the left (toward the center of the instrument). The side with the 6 fluidic ports should face toward the right.

Close the clamp fully. Spring tension will keep the handle pulled toward the flow cell.

Running the Assay

Using the stylus, enter an Assay ID on the first page of the user interface. Touch the space under “Assay ID” using the special PC stylus. A keyboard will appear. Enter any identifier desired, from 3 to 24 characters. Note that the shift key can be activated if desired. The Assay ID will become a file name consisting of the identifier, the current date and the current time for reporting purposes. See FIGS. 7-8.

The default choice on the user interface is to “Run Assay”. This box should remain checked unless the instrument is being cleaned.

Begin Testing by pressing “Enter”. The instrument checks to make sure a flow cell is inserted correctly. If not, a message to the user appears. If an assay flow cell is present, the message “Program Loading . . . ” will appear.

The first program step is to prewet the system with running buffer. This also solubilizes the control antigens inside the flow cell, and allows them to flow into the test array. This filling step takes approximately 90 seconds. At this point, all pumps and valves close, and the user is prompted to inject the sample.

Injecting the Sample

To inject the sample, first remove the white luer connector cap from the flow cell. Attach a syringe containing up to 1 mL of sample.

Slowly inject the sample into the instrument. Leave the syringe attached to the flow cell. In general, a very slow injection is preferable to quick injection.

Assay Progression

Although no user input is required, the OQO computer screen gives a continuous readout of assay progress. The steps are:

Prewetting (described above)

Circulation of antigen over array—20 minutes.

Short air purge of fluid lines

Injection of RLS-antibody reagent—90 seconds

Real-time monitoring of reaction—20 minutes

Final line purge with high-contrast developer—1 minute

Display of Results

Decontamination cycle (after user presses “Enter”)—10 minutes

In the Real-time monitoring, the reaction is monitored for the development of a positive response. When a positive is detected, it is displayed on the computer screen. As time progresses, additional positives may appear. Since the flow cells remains in the optical path all during the assay, the development of RLS signal can be monitored at will, and the user notified as soon as a positive is detected.

Decontamination

The final step, after results are displayed, is the decontamination cycle. Bleach is circulated through the system, followed by a wash with water. Decontamination must be started by user input—press “Enter” when requested.

After decontamination is complete, the flow cell with the attached syringe may be removed and discarded appropriately. Remember that the cell and syringe may be contaminated. The Reservoir Pack can be discarded. Since the waste material is collected (as a gel) in the reservoir pack, it is also contaminated waste, and should be disposed of properly.

Results File and Printing Reports

The assay results are stored in two files on the OQO computer in HTML and JPEG formats. The results file is in the directory C:\MAPPDS. The file names include the Assay ID that was entered by the user, combined with the date and time of the assay (taken from the OQO computer controller clock).

The two files are:

An HTML file of the results, which will open in any browser (e.g., Internet Explorer, FireFox), which includes the results of the assay, and a picture of the array. The file includes identifying information, such as the Assay ID, as well as a GPS-determined location, if available. Current technology limits the use of the GPS feature to outdoor or vehicle use. It is often impossible to get an adequate GPS signal inside a building.

A JPEG file of the array at the end of the testing period, which is called by the HTML file to make the report.

The files can be accessed most easily using the built-in WiFi wireless networking capabilities of the OQO computer controller. This can be easily set up as a secure, peer-to-peer connection to any laptop or desktop computer by anyone well-versed in Microsoft Windows networking technology. It is recommended that only the C:\MAPPDS directory be shared, for security reasons. Both the HTML file and the corresponding JPEG file must be downloaded for each assay, in order to obtain a complete report with an image.

Data Interpretation

The data in the MAPPDS directory are straightforward. The report contains information about the assay, including the assay name, time and date, and MAPP-DS™ instrument used, as well as the GPS location. Each test is graded as “Positive”, “Negative” or “Insufficient Data”. Exemplary JPEG file image for the 6 antigens tested is provided in FIG. 23.

Assay Scoring

The algorithm for assay scoring is described below. Each analyte is tested using results from three independent microfluidic paths: a sample path (top row), a negative control path (middle row), and a positive control path (bottom row). For each analyte, four independent capture array spots are evaluated. Outliers (3 SD from the mean) are eliminated.

The assay score is determined as the percentage of the distance between positive and negative controls at which the unknown falls, as a measurement of the resonance light scattering pixel counts of the array spots. This percentage is calculated as:

Sample Count−Negative Count×100

Positive Count−Negative Count

For example, if the mean pixel count for the positive control is 1000, and the count for the negative is 200, this establishes the span of values to evaluate the unknown. An unknown with a count of 450 would score as [(450−200)/(1000−200)]×100=31.25. This is the score displayed on the screen. The interpretation of “positive” or “negative” has been arbitrarily set at 20%, to minimize false positives. This threshold may change as more assay experience is accumulated. Some of the sensitivity of the assay is sacrificed with such a high threshold, but for biodefense, it is crucially important to minimize false positives. It should be noted that scores greater than 100 percent are possible (stronger signal in the sample than in the positive control).

The JPEG file gives a visual confirmation of the assay result. In most cases, the information in the top part of the report will serve as a “Yes/No” decision for the presence of the agent, which can be followed up with more comprehensive quantitative testing.

Shutdown and Clean-Up. Remove and discard the Flow Cell and the Reagent Pack. Use proper disposal techniques for possible pathogenic material. If performing additional assays, insert a new Flow Cell and a new Reagent Pack . . . If finished, insert a Rinse Cell and Rinse Pack, and run the Cleaning Program (see Example 13 below).

To Shut-Down the computer click “Exit” twice to close the program. Click on Start, then “Turn Off Computer” at the bottom left of the screen. When the exit message pops up, click “Turn Off”. This is the standard Windows exit procedure. Turn the power switch, AC/Off/DC, to “Off”. This is very important. If the switch is left on, it could result in damage to the MAPP-DS™ instrument.

To recharge the battery, first shut down the OQO computer controller, then turn the AC/Off/DC switch to OFF and wait 10 seconds. With the MAPP-DS™ plugged into an AC line turn the switch to the AC position to charge the battery.

### Example 13

### Cleaning the Instrument, Post-Operation

The proper operation of the MAPP-DS™ Instrument requires that periodic cleaning be performed to keep the microfluidic passages clear, and to prevent growth of microorganisms in the fluidics lines. The cleaning protocol involves sequential rinsing of each fluidic path in the instrument. It is recommended that cleaning be done after every 5 assays, or daily (usually at the end of the day).

The cleaning operation requires

A Cleaning Flow Cell, provided with the MAPP-DS™ instrument.

A Cleaning Pack, consisting of a single-use tray filled with appropriate cleaning solutions. The front compartments contain water with, for example, 0.1% Tween-20 detergent. The smaller rear compartment contains water. All compartments are supplemented with an antimicrobial, preferably about 0.2% Bronidox (5-Bromo-5-Nitro-1,3-Dioxane).

Cleaning Protocol.

Start the MAPP-DS™ instrument according to the usual procedure, noted above. Turn on the power switch (AC or DC), turning on the OQO computer controller.

Check the box marked “Run Cleaning” on the User Interface.

Place a Cleaning Flow Cell in the Flow Cell Receiver and close the clamp.

Place a Cleaning Pack in the Reagent Receiver, and push down the lid with two hands until it locks down.

Touch “Enter” and follow the prompts on the screen.

The cleaning cycle is described on the OQO controller screen as the procedure progresses. Basically, the cycle is as follows:

Rinse the buffer lines with water+Tween-20

Rinse the RLS-Antibody lines with water+Tween-20

Rinse the Developer lines with water+Tween-20

Rinse the Bleach lines with water+Tween-20

Rinse the internal manifolds with Water, and flush with air

Repeat #5 for three cycles.

Purge all lines with air.

At the end of the cycle, discard the Cleaning Pack from the Reservoir Receiver. Wipe any drops of liquid that may have spilled into the Reservoir Receiver and any liquid on the underside of the lid.

Remove the Cleaning Flow Cell from the Receiver, and keep it for the next cleaning. Wipe up any drops of liquid in the Flow Cell Receiver Slot, using a cotton swab.

### Example 14

### MAPP-DS™ Troubleshooting

### Computer Issues

A problem which may occasionally arise is a “freeze-up”, a situation where the OQO computer controller no longer responds to pen or keyboard input. The OQO can usually be restored by following this sequence of instructions:

Open the OQO computer to expose the manual keyboard.

Push “FN”, “CTL” and “ALT” keys on the bottom row. A green light will indicate that these keys are being “held” in a pressed state.

Push the “BSP” key at the upper right side of the keyboard. This sequence of keystrokes will bring up the Windows Security display. Click on the “Task Manger” button.

In the “Processes” tab, highlight “BDSProto.exe”, and click “End Process”. Respond “OK” to the confirmation message. Close the window.

You should now be returned to the desktop. Double-click on the shortcut to “BDSProto.exe”, and the program will restart.

Spills and Leaks

The MAPP-DS™ machine is sealed, and has no user-serviceable parts inside. On occasion, material may spill and enter the case. Small spills can be effectively cleaned by following this sequence of instructions:

Elevate the front left corner (under the OQO computer controller) of the MAPP-DS™ instrument about two to three inches (5-8 cm) by placing a block under the corner. This will direct liquid spills to the right rear of the instrument.

Prepare a catheter or tube connected to a syringe or vacuum flask. The tube must be able to fit through the ventilation holes at the rear edge of the instrument.

Snake the tube down one of the holes and direct it to the corner of the case.

Using suction, remove spilled fluid.

If necessary to decontaminate the machine, slowly inject up to 10 ml of household bleach so that it pools in the corner. After a suitable time period, remove the bleach by suction.

Thoroughly wash the case using at least 3 10-ml changes of distilled water.

Let the MAPP-DS™ device dry thoroughly before using.

1. An assay chamber for detecting at least one agent, the assay chamber comprising:
a) a superstructure comprised of a least two circulation ports;
b) a waveguide element comprising at least one binding molecule that binds the at least one agent; and
c) an adhesive means for attaching the base to the waveguide element,
wherein at least one channel is formed between the base and waveguide and wherein the at least two circulation ports and the at least one binding molecule are aligned with the at least one channel.

2. The assay chamber of claim 1, wherein the number of channels is selected from the group consisting of 2, 3, 4, 5, 6, 7, 8, 9 and 10.

3. The assay chamber of claim 1, wherein the base comprises a number of circulation ports selected from the group consisting of 3, 4, 5, 6, 7, 8, 9, 10, 11, 12, 13, 14, 15, 16, 17, 18, 19 and 20.

4. The assay chamber of claim 1, further comprising a computer readable label.

5. The assay chamber of claim 1, wherein the waveguide element comprises an opaque mask, wherein the mask is not present in line with at least one situs comprising the at least one binding molecule.

6. The assay chamber of claim 1, wherein the at least one binding molecule comprises a first binding molecule that is capable of binding a first agent and a second binding molecule that is capable of binding a second agent.

7. A method of detecting at least one agent in a sample comprising:
a) contacting the sample or a fraction of the sample with the at least one binding molecule of claim 1;
b) contacting the sample or a fraction of the sample with a detector binding molecule, wherein the detector binding molecule is directly or indirectly associated with a label;
c) detecting a signal from the label; and
d) correlating the detectable signal with the detection of the at least one agent.

8. A method for detecting at least two agents in a sample comprising:
a) contacting the sample or a fraction of the sample with the first and second binding molecules of claim 6;
b) contacting the sample or a fraction of the sample with a first detector binding molecule that is capable of binding a first agent and a second detector binding molecule that is capable of binding a second agent, wherein the first and second detector binding molecules are directly or indirectly associated with a label;
c) detecting a signal from the label; and
d) correlating the detectable signal or signals with the detection of the at least two agents.

9. The method of claim 8 wherein, the first detector binding molecule is labeled with a first label and the second detector binding molecule is labeled with a second label and wherein the first and second label produce distinguishable signals.

10. A method of measuring, detecting or monitoring a binding interaction between the at least one binding molecule and the at least one agent of claim 1 comprising:
a) contacting the at least one agent with the at least one binding molecule, wherein the at least one agent is directly or indirectly labeled;
c) detecting a signal from the label; and
d) correlating the detectable signal with the binding interaction

11. The method of claim 10, wherein the detecting is performed once.

12. The method of claim 10, wherein the detecting is performed at multiple times to produce multiple detectable signals and the multiple detectable signals are correlated with the binding interaction.

13. A device for holding the assay chamber of claim 1 during a detection assay wherein the device allows reagents to circulate through the at least two circulation ports.

14. The device of claim 13, comprising a switch for detecting proper insertion of an assay chamber.

15. The device of claim 13, comprising a clamp mechanism that connects ports in the clamp mechanism to the at least two circulation ports, wherein the connection is made via an O-ring.

16. A detection apparatus for performing at least one detection assay comprising a reagent reservoir delivery unit, wherein the reagent reservoir delivery unit is designed to access a reagent in a reagent pack, wherein the reagent pack comprises at least one reservoir and wherein upon proper insertion of the reagent pack into the reagent reservoir delivery unit, ports are automatically inserted into the at least one reservoir of the reagent pack.

17. The detection apparatus of claim 16, wherein the reagent pack is a blister pack.

18. The detection apparatus of claim 17, wherein the automatic insertion of ports comprises the ports piercing the blister pack.

19. The detection apparatus of claim 16, wherein the reagent pack comprises a number of reservoirs selected from the group consisting of 2, 3, 4, 5, 6, 7, 8, 9 and 10.

20. An apparatus for the analysis of an agent in a sample comprising an assay chamber, a light source, a detection device, and a computer wherein the apparatus is capable of being manually carried by an average adult.

21. The apparatus of claim 20, further comprising at least one component selected from the group consisting of, at least one global positioning system receiver, at least one pump for fluids, a reagent pack holder, a reagent pack, a battery, and a flow cell clamp device.

22. A reagent pack, wherein the reagent pack comprises at least two reservoirs, wherein at least one of the reservoirs comprises a reagent and wherein at least one reservoir comprises a lid.

23. The reagent pack of claim 22, wherein the lid is capable of being pierced by tubes or ports present in the device or apparatus.

24. The reagent pack of claim 22, wherein at least one of the at least two reservoirs is designed to collect reagents from the device or apparatus.

25. The reagent pack of claim 22, wherein the reagent pack comprises all of the necessary reagents or at least one of the necessary reagents for the device or apparatus.
